SEQUENCE LISTING (1159 sequences)

SEQ ID NO: 1 - DNA, 10 bp, synthetic construct
  source 1..10: mol_type = other DNA
ccgttgtgag

SEQ ID NO: 2 - DNA, 10 bp, synthetic construct
  source 1..10: mol_type = other DNA
aatggtgagt

SEQ ID NO: 3 - DNA, 10 bp, synthetic construct
  source 1..10: mol_type = other DNA
tgcaaccttc

SEQ ID NO: 4 - DNA, 10 bp, synthetic construct
  source 1..10: mol_type = other DNA
gtaccaacca

SEQ ID NO: 5 - DNA, 10 bp, synthetic construct
  source 1..10: mol_type = other DNA
tgcgttctgc

SEQ ID NO: 6 - DNA, 10 bp, synthetic construct
  source 1..10: mol_type = other DNA
ctgtcgtgat

SEQ ID NO: 7 - DNA, 10 bp, synthetic construct
  source 1..10: mol_type = other DNA
acaagcaatg

SEQ ID NO: 8 - DNA, 10 bp, synthetic construct
  source 1..10: mol_type = other DNA
gatcaagcca

SEQ ID NO: 9 - DNA, 10 bp, synthetic construct
  source 1..10: mol_type = other DNA
acttagatcg

SEQ ID NO: 10 - DNA, 10 bp, synthetic construct
  source 1..10: mol_type = other DNA
ttaggaggaa

SEQ ID NO: 11 - DNA, 10 bp, synthetic construct
  source 1..10: mol_type = other DNA
cgcatccatc

SEQ ID NO: 12 - DNA, 10 bp, synthetic construct
  source 1..10: mol_type = other DNA
gagccttcgt

SEQ ID NO: 13 - DNA, 10 bp, synthetic construct
  source 1..10: mol_type = other DNA
actcgcggta

SEQ ID NO: 14 - DNA, 10 bp, synthetic construct
  source 1..10: mol_type = other DNA
cacgaacact

SEQ ID NO: 15 - DNA, 10 bp, synthetic construct
  source 1..10: mol_type = other DNA
gtaactatgg

SEQ ID NO: 16 - DNA, 10 bp, synthetic construct
  source 1..10: mol_type = other DNA
tggttgtcac

SEQ ID NO: 17 - DNA, 10 bp, synthetic construct
  source 1..10: mol_type = other DNA
aggtcactag

SEQ ID NO: 18 - DNA, 10 bp, synthetic construct
  source 1..10: mol_type = other DNA
cctgatagcc

SEQ ID NO: 19 - DNA, 10 bp, synthetic construct
  source 1..10: mol_type = other DNA
gacctggatt

SEQ ID NO: 20 - DNA, 10 bp, synthetic construct
  source 1..10: mol_type = other DNA
ttaagctcga

SEQ ID NO: 21 - DNA, 10 bp, synthetic construct
  source 1..10: mol_type = other DNA
cggtgaagtc

SEQ ID NO: 22 - DNA, 10 bp, synthetic construct
  source 1..10: mol_type = other DNA
gtaactgcat

SEQ ID NO: 23 - DNA, 10 bp, synthetic construct
  source 1..10: mol_type = other DNA
tctctgttga

SEQ ID NO: 24 - DNA, 10 bp, synthetic construct
  source 1..10: mol_type = other DNA
aacgaccacg

SEQ ID NO: 25 - DNA, 10 bp, synthetic construct
  source 1..10: mol_type = other DNA
gcgaatcagc

SEQ ID NO: 26 - DNA, 10 bp, synthetic construct
  source 1..10: mol_type = other DNA
aacttcggta

SEQ ID NO: 27 - DNA, 10 bp, synthetic construct
  source 1..10: mol_type = other DNA
tgtggaatag

SEQ ID NO: 28 - DNA, 10 bp, synthetic construct
  source 1..10: mol_type = other DNA
ctaccgtcct

SEQ ID NO: 29 - DNA, 10 bp, synthetic construct
  source 1..10: mol_type = other DNA
ccataagagg

SEQ ID NO: 30 - DNA, 10 bp, synthetic construct
  source 1..10: mol_type = other DNA
gtcggtagtc

SEQ ID NO: 31 - DNA, 10 bp, synthetic construct
  source 1..10: mol_type = other DNA
tgtctgccat

SEQ ID NO: 32 - DNA, 10 bp, synthetic construct
  source 1..10: mol_type = other DNA
aagaccttca

SEQ ID NO: 33 - DNA, 10 bp, synthetic construct
  source 1..10: mol_type = other DNA
ccgattcgat

SEQ ID NO: 34 - DNA, 10 bp, synthetic construct
  source 1..10: mol_type = other DNA
attgcagcca

SEQ ID NO: 35 - DNA, 10 bp, synthetic construct
  source 1..10: mol_type = other DNA
gaatacttgc

SEQ ID NO: 36 - DNA, 10 bp, synthetic construct
  source 1..10: mol_type = other DNA
tgccggaatg

SEQ ID NO: 37 - DNA, 10 bp, synthetic construct
  source 1..10: mol_type = other DNA
atgcggatcc

SEQ ID NO: 38 - DNA, 10 bp, synthetic construct
  source 1..10: mol_type = other DNA
ccttcatgaa

SEQ ID NO: 39 - DNA, 10 bp, synthetic construct
  source 1..10: mol_type = other DNA
gacgatcatg

SEQ ID NO: 40 - DNA, 10 bp, synthetic construct
  source 1..10: mol_type = other DNA
tgaatcgcgt

SEQ ID NO: 41 - DNA, 10 bp, synthetic construct
  source 1..10: mol_type = other DNA
tacactcggt

SEQ ID NO: 42 - DNA, 10 bp, synthetic construct
  source 1..10: mol_type = other DNA
atacaggacg

SEQ ID NO: 43 - DNA, 10 bp, synthetic construct
  source 1..10: mol_type = other DNA
cgtgtctcac

SEQ ID NO: 44 - DNA, 10 bp, synthetic construct
  source 1..10: mol_type = other DNA
gcgtgaatta

SEQ ID NO: 45 - DNA, 10 bp, synthetic construct
  source 1..10: mol_type = other DNA
ttcgttcaca

SEQ ID NO: 46 - DNA, 10 bp, synthetic construct
  source 1..10: mol_type = other DNA
gagacggttg

SEQ ID NO: 47 - DNA, 10 bp, synthetic construct
  source 1..10: mol_type = other DNA
acacaatggt

SEQ ID NO: 48 - DNA, 10 bp, synthetic construct
  source 1..10: mol_type = other DNA
cgttgcacac

SEQ ID NO: 49 - DNA, 10 bp, synthetic construct
  source 1..10: mol_type = other DNA
gaacactatc

SEQ ID NO: 50 - DNA, 10 bp, synthetic construct
  source 1..10: mol_type = other DNA
tctgcggtaa

SEQ ID NO: 51 - DNA, 10 bp, synthetic construct
  source 1..10: mol_type = other DNA
aggttaagcg

SEQ ID NO: 52 - DNA, 10 bp, synthetic construct
  source 1..10: mol_type = other DNA
ctcagtccgt

SEQ ID NO: 53 - DNA, 10 bp, synthetic construct
  source 1..10: mol_type = other DNA
cgcaatgaac

SEQ ID NO: 54 - DNA, 10 bp, synthetic construct
  source 1..10: mol_type = other DNA
ttggcactta

SEQ ID NO: 55 - DNA, 10 bp, synthetic construct
  source 1..10: mol_type = other DNA
gcattcagcg

SEQ ID NO: 56 - DNA, 10 bp, synthetic construct
  source 1..10: mol_type = other DNA
aatcggtcgt

SEQ ID NO: 57 - DNA, 10 bp, synthetic construct
  source 1..10: mol_type = other DNA
atatggcaga

SEQ ID NO: 58 - DNA, 10 bp, synthetic construct
  source 1..10: mol_type = other DNA
cggactgtct

SEQ ID NO: 59 - DNA, 10 bp, synthetic construct
  source 1..10: mol_type = other DNA
gatcaatctg

SEQ ID NO: 60 - DNA, 10 bp, synthetic construct
  source 1..10: mol_type = other DNA
tccgtcagac

SEQ ID NO: 61 - DNA, 10 bp, synthetic construct
  source 1..10: mol_type = other DNA
agagtgcatg

SEQ ID NO: 62 - DNA, 10 bp, synthetic construct
  source 1..10: mol_type = other DNA
gttcgcatac

SEQ ID NO: 63 - DNA, 10 bp, synthetic construct
  source 1..10: mol_type = other DNA
tcgaattcgt

SEQ ID NO: 64 - DNA, 10 bp, synthetic construct
  source 1..10: mol_type = other DNA
cactcaggca

SEQ ID NO: 65 - DNA, 10 bp, synthetic construct
  source 1..10: mol_type = other DNA
ccttggtgca

SEQ ID NO: 66 - DNA, 10 bp, synthetic construct
  source 1..10: mol_type = other DNA
gagcatccat

SEQ ID NO: 67 - DNA, 10 bp, synthetic construct
  source 1..10: mol_type = other DNA
agagtcgatc

SEQ ID NO: 68 - DNA, 10 bp, synthetic construct
  source 1..10: mol_type = other DNA
ttcacaatgg

SEQ ID NO: 69 - DNA, 10 bp, synthetic construct
  source 1..10: mol_type = other DNA
gtatcctcag

SEQ ID NO: 70 - DNA, 10 bp, synthetic construct
  source 1..10: mol_type = other DNA
taccgagtgt

SEQ ID NO: 71 - DNA, 10 bp, synthetic construct
  source 1..10: mol_type = other DNA
actgtgcacc

SEQ ID NO: 72 - DNA, 10 bp, synthetic construct
  source 1..10: mol_type = other DNA
cggaatagta

SEQ ID NO: 73 - DNA, 10 bp, synthetic construct
  source 1..10: mol_type = other DNA
gtgtctacag

SEQ ID NO: 74 - DNA, 10 bp, synthetic construct
  source 1..10: mol_type = other DNA
tacgtatacc

SEQ ID NO: 75 - DNA, 10 bp, synthetic construct
  source 1..10: mol_type = other DNA
agtaagcggt

SEQ ID NO: 76 - DNA, 10 bp, synthetic construct
  source 1..10: mol_type = other DNA
ccacgcgtta

SEQ ID NO: 77 - DNA, 10 bp, synthetic construct
  source 1..10: mol_type = other DNA
agcttatggc

SEQ ID NO: 78 - DNA, 10 bp, synthetic construct
  source 1..10: mol_type = other DNA
ccgaatgatt

SEQ ID NO: 79 - DNA, 10 bp, synthetic construct
  source 1..10: mol_type = other DNA
gttggcacca

SEQ ID NO: 80 - DNA, 10 bp, synthetic construct
  source 1..10: mol_type = other DNA
taaccgctag

SEQ ID NO: 81 - DNA, 10 bp, synthetic construct
  source 1..10: mol_type = other DNA
agaagtagga

SEQ ID NO: 82 - DNA, 10 bp, synthetic construct
  source 1..10: mol_type = other DNA
cttccagttg

SEQ ID NO: 83 - DNA, 10 bp, synthetic construct
  source 1..10: mol_type = other DNA
gaggtgcaac

SEQ ID NO: 84 - DNA, 10 bp, synthetic construct
  source 1..10: mol_type = other DNA
tcctactcct

SEQ ID NO: 85 - DNA, 10 bp, synthetic construct
  source 1..10: mol_type = other DNA
tccgacagtt

SEQ ID NO: 86 - DNA, 10 bp, synthetic construct
  source 1..10: mol_type = other DNA
agtagttacc

SEQ ID NO: 87 - DNA, 10 bp, synthetic construct
  source 1..10: mol_type = other DNA
gaatcggtgg

SEQ ID NO: 88 - DNA, 10 bp, synthetic construct
  source 1..10: mol_type = other DNA
ctgctaccaa

SEQ ID NO: 89 - DNA, 10 bp, synthetic construct
  source 1..10: mol_type = other DNA
cgttgaatgg

SEQ ID NO: 90 - DNA, 10 bp, synthetic construct
  source 1..10: mol_type = other DNA
atggcttcta

SEQ ID NO: 91 - DNA, 10 bp, synthetic construct
  source 1..10: mol_type = other DNA
gacatgcacc

SEQ ID NO: 92 - DNA, 10 bp, synthetic construct
  source 1..10: mol_type = other DNA
tcacacggat

SEQ ID NO: 93 - DNA, 10 bp, synthetic construct
  source 1..10: mol_type = other DNA
ccaatatggt

SEQ ID NO: 94 - DNA, 10 bp, synthetic construct
  source 1..10: mol_type = other DNA
gtctcgctca

SEQ ID NO: 95 - DNA, 10 bp, synthetic construct
  source 1..10: mol_type = other DNA
tggcacgaac

SEQ ID NO: 96 - DNA, 10 bp, synthetic construct
  source 1..10: mol_type = other DNA
aatggtactg

SEQ ID NO: 97 - DNA, 10 bp, synthetic construct
  source 1..10: mol_type = other DNA
gaggctgaat

SEQ ID NO: 98 - DNA, 10 bp, synthetic construct
  source 1..10: mol_type = other DNA
agtaagagtc

SEQ ID NO: 99 - DNA, 10 bp, synthetic construct
  source 1..10: mol_type = other DNA
tcctgactga

SEQ ID NO: 100 - DNA, 10 bp, synthetic construct
  source 1..10: mol_type = other DNA
ctactctccg

SEQ ID NO: 101 - DNA, 10 bp, synthetic construct
  source 1..10: mol_type = other DNA
aggtctgatt

SEQ ID NO: 102 - DNA, 10 bp, synthetic construct
  source 1..10: mol_type = other DNA
cttgagccag

SEQ ID NO: 103 - DNA, 10 bp, synthetic construct
  source 1..10: mol_type = other DNA
gaacgaaggc

SEQ ID NO: 104 - DNA, 10 bp, synthetic construct
  source 1..10: mol_type = other DNA
tccatcttca

SEQ ID NO: 105 - DNA, 10 bp, synthetic construct
  source 1..10: mol_type = other DNA
cgtcacgcaa

SEQ ID NO: 106 - DNA, 10 bp, synthetic construct
  source 1..10: mol_type = other DNA
gcctgttatt

SEQ ID NO: 107 - DNA, 10 bp, synthetic construct
  source 1..10: mol_type = other DNA
ataacaagcg

SEQ ID NO: 108 - DNA, 10 bp, synthetic construct
  source 1..10: mol_type = other DNA
taggtgctgc

SEQ ID NO: 109 - DNA, 10 bp, synthetic construct
  source 1..10: mol_type = other DNA
gatctgaaca

SEQ ID NO: 110 - DNA, 10 bp, synthetic construct
  source 1..10: mol_type = other DNA
ctcaatgtgt

SEQ ID NO: 111 - DNA, 10 bp, synthetic construct
  source 1..10: mol_type = other DNA
tcgtgacgac

SEQ ID NO: 112 - DNA, 10 bp, synthetic construct
  source 1..10: mol_type = other DNA
agagcctctg

SEQ ID NO: 113 - DNA, 10 bp, synthetic construct
  source 1..10: mol_type = other DNA
cgaagtaccg

SEQ ID NO: 114 - DNA, 10 bp, synthetic construct
  source 1..10: mol_type = other DNA
accgaataga

SEQ ID NO: 115 - DNA, 10 bp, synthetic construct
  source 1..10: mol_type = other DNA
tagtcgctat

SEQ ID NO: 116 - DNA, 10 bp, synthetic construct
  source 1..10: mol_type = other DNA
gttctcggtc

SEQ ID NO: 117 - DNA, 10 bp, synthetic construct
  source 1..10: mol_type = other DNA
aacacgagaa

SEQ ID NO: 118 - DNA, 10 bp, synthetic construct
  source 1..10: mol_type = other DNA
cttcgctctc

SEQ ID NO: 119 - DNA, 10 bp, synthetic construct
  source 1..10: mol_type = other DNA
gcgtatctcg

SEQ ID NO: 120 - DNA, 10 bp, synthetic construct
  source 1..10: mol_type = other DNA
tgagtagagt

SEQ ID NO: 121 - DNA, 10 bp, synthetic construct
  source 1..10: mol_type = other DNA
tggagttgcg

SEQ ID NO: 122 - DNA, 10 bp, synthetic construct
  source 1..10: mol_type = other DNA
gttgtaacgt

SEQ ID NO: 123 - DNA, 10 bp, synthetic construct
  source 1..10: mol_type = other DNA
acctacctaa

SEQ ID NO: 124 - DNA, 10 bp, synthetic construct
  source 1..10: mol_type = other DNA
caaccggatc

SEQ ID NO: 125 - DNA, 10 bp, synthetic construct
  source 1..10: mol_type = other DNA
gatagcttcg

SEQ ID NO: 126 - DNA, 10 bp, synthetic construct
  source 1..10: mol_type = other DNA
ccgcaacgat

SEQ ID NO: 127 - DNA, 10 bp, synthetic construct
  source 1..10: mol_type = other DNA
agatcggata

SEQ ID NO: 128 - DNA, 10 bp, synthetic construct
  source 1..10: mol_type = other DNA
ttcgttacgc

SEQ ID NO: 129 - DNA, 10 bp, synthetic construct
  source 1..10: mol_type = other DNA
aagagacaca

SEQ ID NO: 130 - DNA, 10 bp, synthetic construct
  source 1..10: mol_type = other DNA
ccacttggat

SEQ ID NO: 131 - DNA, 10 bp, synthetic construct
  source 1..10: mol_type = other DNA
gtctccttgc

SEQ ID NO: 132 - DNA, 10 bp, synthetic construct
  source 1..10: mol_type = other DNA
tgtgagactg

SEQ ID NO: 133 - DNA, 10 bp, synthetic construct
  source 1..10: mol_type = other DNA
aggatagccg

SEQ ID NO: 134 - DNA, 10 bp, synthetic construct
  source 1..10: mol_type = other DNA
tctgctatac

SEQ ID NO: 135 - DNA, 10 bp, synthetic construct
  source 1..10: mol_type = other DNA
gtctactggt

SEQ ID NO: 136 - DNA, 10 bp, synthetic construct
  source 1..10: mol_type = other DNA
caacggcata

SEQ ID NO: 137 - DNA, 10 bp, synthetic construct
  source 1..10: mol_type = other DNA
gtacagcgga

SEQ ID NO: 138 - DNA, 10 bp, synthetic construct
  source 1..10: mol_type = other DNA
cgcatagcac

SEQ ID NO: 139 - DNA, 10 bp, synthetic construct
  source 1..10: mol_type = other DNA
actggttatg

SEQ ID NO: 140 - DNA, 10 bp, synthetic construct
  source 1..10: mol_type = other DNA
tagtccatct

SEQ ID NO: 141 - DNA, 10 bp, synthetic construct
  source 1..10: mol_type = other DNA
tgacggttgc

SEQ ID NO: 142 - DNA, 10 bp, synthetic construct
  source 1..10: mol_type = other DNA
gctaaccgtg

SEQ ID NO: 143 - DNA, 10 bp, synthetic construct
  source 1..10: mol_type = other DNA
atggttgact

SEQ ID NO: 144 - DNA, 10 bp, synthetic construct
  source 1..10: mol_type = other DNA
cactcaacaa

SEQ ID NO: 145 - DNA, 10 bp, synthetic construct
  source 1..10: mol_type = other DNA
taagctacca

SEQ ID NO: 146 - DNA, 10 bp, synthetic construct
  source 1..10: mol_type = other DNA
agctgaggtg

SEQ ID NO: 147 - DNA, 10 bp, synthetic construct
  source 1..10: mol_type = other DNA
cttctccagc

SEQ ID NO: 148 - DNA, 10 bp, synthetic construct
  source 1..10: mol_type = other DNA
gcgaagttat

SEQ ID NO: 149 - DNA, 10 bp, synthetic construct
  source 1..10: mol_type = other DNA
atccatgtcc

SEQ ID NO: 150 - DNA, 10 bp, synthetic construct
  source 1..10: mol_type = other DNA
taggtctcgg

SEQ ID NO: 151 - DNA, 10 bp, synthetic construct
  source 1..10: mol_type = other DNA
gcatgaagaa

SEQ ID NO: 152 - DNA, 10 bp, synthetic construct
  source 1..10: mol_type = other DNA
cgtacgcatt

SEQ ID NO: 153 - DNA, 10 bp, synthetic construct
  source 1..10: mol_type = other DNA
gtgaattgac

SEQ ID NO: 154 - DNA, 10 bp, synthetic construct
  source 1..10: mol_type = other DNA
tacctagact

SEQ ID NO: 155 - DNA, 10 bp, synthetic construct
  source 1..10: mol_type = other DNA
agatgcctgg

SEQ ID NO: 156 - DNA, 10 bp, synthetic construct
  source 1..10: mol_type = other DNA
cctgcgacta

SEQ ID NO: 157 - DNA, 10 bp, synthetic construct
  source 1..10: mol_type = other DNA
ccggtcaact

SEQ ID NO: 158 - DNA, 10 bp, synthetic construct
  source 1..10: mol_type = other DNA
gtaagatgag

SEQ ID NO: 159 - DNA, 10 bp, synthetic construct
  source 1..10: mol_type = other DNA
tactcgccga

SEQ ID NO: 160 - DNA, 10 bp, synthetic construct
  source 1..10: mol_type = other DNA
tactcgccga

SEQ ID NO: 161 - DNA, 10 bp, synthetic construct
  source 1..10: mol_type = other DNA
tcgaggttcc

SEQ ID NO: 162 - DNA, 10 bp, synthetic construct
  source 1..10: mol_type = other DNA
gatgctagaa

SEQ ID NO: 163 - DNA, 10 bp, synthetic construct
  source 1..10: mol_type = other DNA
atctaacctg

SEQ ID NO: 164 - DNA, 10 bp, synthetic construct
  source 1..10: mol_type = other DNA
cgactcgagt

SEQ ID NO: 165 - DNA, 10 bp, synthetic construct
  source 1..10: mol_type = other DNA
accatactcc

SEQ ID NO: 166 - DNA, 10 bp, synthetic construct
  source 1..10: mol_type = other DNA
cggtgtacaa

SEQ ID NO: 167 - DNA, 10 bp, synthetic construct
  source 1..10: mol_type = other DNA
gttgcggagt

SEQ ID NO: 168 - DNA, 10 bp, synthetic construct
  source 1..10: mol_type = other DNA
taacactgtg

SEQ ID NO: 169 - DNA, 10 bp, synthetic construct
  source 1..10: mol_type = other DNA
caagtaacgg

SEQ ID NO: 170 - DNA, 10 bp, synthetic construct
  source 1..10: mol_type = other DNA
tcgagtgtat

SEQ ID NO: 171 - DNA, 10 bp, synthetic construct
  source 1..10: mol_type = other DNA
gttccgtacc

SEQ ID NO: 172 - DNA, 10 bp, synthetic construct
  source 1..10: mol_type = other DNA
agctaccgta

SEQ ID NO: 173 - DNA, 10 bp, synthetic construct
  source 1..10: mol_type = other DNA
tgatacgcgg

SEQ ID NO: 174 - DNA, 10 bp, synthetic construct
  source 1..10: mol_type = other DNA
ccgacacatc

SEQ ID NO: 175 - DNA, 10 bp, synthetic construct
  source 1..10: mol_type = other DNA
gttggttgaa

SEQ ID NO: 176 - DNA, 10 bp, synthetic construct
  source 1..10: mol_type = other DNA
aacctgatct

SEQ ID NO: 177 - DNA, 10 bp, synthetic construct
  source 1..10: mol_type = other DNA
attgagcact

SEQ ID NO: 178 - DNA, 10 bp, synthetic construct
  source 1..10: mol_type = other DNA
tgaattgtgc

SEQ ID NO: 179 - DNA, 10 bp, synthetic construct
  source 1..10: mol_type = other DNA
gaccgatgtg

SEQ ID NO: 180 - DNA, 10 bp, synthetic construct
  source 1..10: mol_type = other DNA
ccgtccacaa

SEQ ID NO: 181 - DNA, 10 bp, synthetic construct
  source 1..10: mol_type = other DNA
taagacgcca

SEQ ID NO: 182 - DNA, 10 bp, synthetic construct
  source 1..10: mol_type = other DNA
ctctctctag

SEQ ID NO: 183 - DNA, 10 bp, synthetic construct
  source 1..10: mol_type = other DNA
gctagatagt

SEQ ID NO: 184 - DNA, 10 bp, synthetic construct
  source 1..10: mol_type = other DNA
aggctgagtc

SEQ ID NO: 185 - DNA, 10 bp, synthetic construct
  source 1..10: mol_type = other DNA
tctaacgtgt

SEQ ID NO: 186 - DNA, 10 bp, synthetic construct
  source 1..10: mol_type = other DNA
aggcctccaa

SEQ ID NO: 187 - DNA, 10 bp, synthetic construct
  source 1..10: mol_type = other DNA
gaattgaacc

SEQ ID NO: 188 - DNA, 10 bp, synthetic construct
  source 1..10: mol_type = other DNA
ctcggatgtg

SEQ ID NO: 189 - DNA, 10 bp, synthetic construct
  source 1..10: mol_type = other DNA
gttctaactc

SEQ ID NO: 190 - DNA, 10 bp, synthetic construct
  source 1..10: mol_type = other DNA
ccaactcgct

SEQ ID NO: 191 - DNA, 10 bp, synthetic construct
  source 1..10: mol_type = other DNA
aggtagtaag

SEQ ID NO: 192 - DNA, 10 bp, synthetic construct
  source 1..10: mol_type = other DNA
tacggcgtga

SEQ ID NO: 193 - DNA, 10 bp, synthetic construct
  source 1..10: mol_type = other DNA
gctctgtgtc

SEQ ID NO: 194 - DNA, 10 bp, synthetic construct
  source 1..10: mol_type = other DNA
tggtgcgcaa

SEQ ID NO: 195 - DNA, 10 bp, synthetic construct
  source 1..10: mol_type = other DNA
aacacactgt

SEQ ID NO: 196 - DNA, 10 bp, synthetic construct
  source 1..10: mol_type = other DNA
ctagataacg

SEQ ID NO: 197 - DNA, 10 bp, synthetic construct
  source 1..10: mol_type = other DNA
aggccgttac

SEQ ID NO: 198 - DNA, 10 bp, synthetic construct
  source 1..10: mol_type = other DNA
cttggacctg

SEQ ID NO: 199 - DNA, 10 bp, synthetic construct
  source 1..10: mol_type = other DNA
tccaatagga

SEQ ID NO: 200 - DNA, 10 bp, synthetic construct
  source 1..10: mol_type = other DNA
gaattcgact

SEQ ID NO: 201 - DNA, 10 bp, synthetic construct
  source 1..10: mol_type = other DNA
gaattcgcga

SEQ ID NO: 202 - DNA, 10 bp, synthetic construct
  source 1..10: mol_type = other DNA
cgtggtcact

SEQ ID NO: 203 - DNA, 10 bp, synthetic construct
  source 1..10: mol_type = other DNA
ttcacgatac

SEQ ID NO: 204 - DNA, 10 bp, synthetic construct
  source 1..10: mol_type = other DNA
acgcaatgtg

SEQ ID NO: 205 - DNA, 10 bp, synthetic construct
  source 1..10: mol_type = other DNA
acaatccggc

SEQ ID NO: 206 - DNA, 10 bp, synthetic construct
  source 1..10: mol_type = other DNA
ttgtggtatg

SEQ ID NO: 207 - DNA, 10 bp, synthetic construct
  source 1..10: mol_type = other DNA
cgccatgtaa

SEQ ID NO: 208 - DNA, 10 bp, synthetic construct
  source 1..10: mol_type = other DNA
gatgcaacct

SEQ ID NO: 209 - DNA, 10 bp, synthetic construct
  source 1..10: mol_type = other DNA
tatcgttgtg

SEQ ID NO: 210 - DNA, 10 bp, synthetic construct
  source 1..10: mol_type = other DNA
atgacagcac

SEQ ID NO: 211 - DNA, 10 bp, synthetic construct
  source 1..10: mol_type = other DNA
cgcttcatgt

SEQ ID NO: 212 - DNA, 10 bp, synthetic construct
  source 1..10: mol_type = other DNA
gcagagcaca

SEQ ID NO: 213 - DNA, 10 bp, synthetic construct
  source 1..10: mol_type = other DNA
ctacgcgaag

SEQ ID NO: 214 - DNA, 10 bp, synthetic construct
  source 1..10: mol_type = other DNA
aaggtgattc

SEQ ID NO: 215 - DNA, 10 bp, synthetic construct
  source 1..10: mol_type = other DNA
gcttattgct

SEQ ID NO: 216 - DNA, 10 bp, synthetic construct
  source 1..10: mol_type = other DNA
tgcacaccga

SEQ ID NO: 217 - DNA, 10 bp, synthetic construct
  source 1..10: mol_type = other DNA
actggatccg

SEQ ID NO: 218 - DNA, 10 bp, synthetic construct
  source 1..10: mol_type = other DNA
gagaacagat

SEQ ID NO: 219 - DNA, 10 bp, synthetic construct
  source 1..10: mol_type = other DNA
ttacctcaga

SEQ ID NO: 220 - DNA, 10 bp, synthetic construct
  source 1..10: mol_type = other DNA
cgcttggttc

SEQ ID NO: 221 - DNA, 10 bp, synthetic construct
  source 1..10: mol_type = other DNA
tcacctctcg

SEQ ID NO: 222 - DNA, 10 bp, synthetic construct
  source 1..10: mol_type = other DNA
atcagcgctc

SEQ ID NO: 223 - DNA, 10 bp, synthetic construct
  source 1..10: mol_type = other DNA
cggtaatgaa

SEQ ID NO: 224 - DNA, 10 bp, synthetic construct
  source 1..10: mol_type = other DNA
gatgtgaagt

SEQ ID NO: 225 - DNA, 10 bp, synthetic construct
  source 1..10: mol_type = other DNA
tagttggtac

SEQ ID NO: 226 - DNA, 10 bp, synthetic construct
  source 1..10: mol_type = other DNA
cgagacaact

SEQ ID NO: 227 - DNA, 10 bp, synthetic construct
  source 1..10: mol_type = other DNA
gttccaccga

SEQ ID NO: 228 - DNA, 10 bp, synthetic construct
  source 1..10: mol_type = other DNA
accagttgtg

SEQ ID NO: 229 - DNA, 10 bp, synthetic construct
  source 1..10: mol_type = other DNA
caagcagcag

SEQ ID NO: 230 - DNA, 10 bp, synthetic construct
  source 1..10: mol_type = other DNA
gcgaactggt

SEQ ID NO: 231 - DNA, 10 bp, synthetic construct
  source 1..10: mol_type = other DNA
tgctgtctta

SEQ ID NO: 232 - DNA, 10 bp, synthetic construct
  source 1..10: mol_type = other DNA
attctgaacc

SEQ ID NO: 233 - DNA, 10 bp, synthetic construct
  source 1..10: mol_type = other DNA
accaatcgtt

SEQ ID NO: 234 - DNA, 10 bp, synthetic construct
  source 1..10: mol_type = other DNA
tgttcagcaa

SEQ ID NO: 235 - DNA, 10 bp, synthetic construct
  source 1..10: mol_type = other DNA
caggtgtagc

SEQ ID NO: 236 - DNA, 10 bp, synthetic construct
  source 1..10: mol_type = other DNA
gtacgcatcg

SEQ ID NO: 237 - DNA, 10 bp, synthetic construct
  source 1..10: mol_type = other DNA
caacaccgag

SEQ ID NO: 238 - DNA, 10 bp, synthetic construct
  source 1..10: mol_type = other DNA
gttgcagatc

SEQ ID NO: 239 - DNA, 10 bp, synthetic construct
  source 1..10: mol_type = other DNA
tggtggttgt

SEQ ID NO: 240 - DNA, 10 bp, synthetic construct
  source 1..10: mol_type = other DNA
accattacca

SEQ ID NO: 241 - DNA, 10 bp, synthetic construct
  source 1..10: mol_type = other DNA
agttgtctgc

SEQ ID NO: 242 - DNA, 10 bp, synthetic construct
  source 1..10: mol_type = other DNA
ctagaggaca

SEQ ID NO: 243 - DNA, 10 bp, synthetic construct
  source 1..10: mol_type = other DNA
gcgacctcat

SEQ ID NO: 244 - DNA, 10 bp, synthetic construct
  source 1..10: mol_type = other DNA
tacctaagtg

SEQ ID NO: 245 - DNA, 10 bp, synthetic construct
  source 1..10: mol_type = other DNA
gtgttatctc

SEQ ID NO: 246 - DNA, 10 bp, synthetic construct
  source 1..10: mol_type = other DNA
tcagacgtcg

SEQ ID NO: 247 - DNA, 10 bp, synthetic construct
  source 1..10: mol_type = other DNA
aaccggaagt

SEQ ID NO: 248 - DNA, 10 bp, synthetic construct
  source 1..10: mol_type = other DNA
cgtactcgaa

SEQ ID NO: 249 - DNA, 10 bp, synthetic construct
  source 1..10: mol_type = other DNA
actcgaaggt

SEQ ID NO: 250 - DNA, 10 bp, synthetic construct
  source 1..10: mol_type = other DNA
cgcttgtaag

SEQ ID NO: 251 - DNA, 10 bp, synthetic construct
  source 1..10: mol_type = other DNA
gtaaccgctc

SEQ ID NO: 252 - DNA, 10 bp, synthetic construct
  source 1..10: mol_type = other DNA
taggatctca

SEQ ID NO: 253 - DNA, 10 bp, synthetic construct
  source 1..10: mol_type = other DNA
gtccgattac

SEQ ID NO: 254 - DNA, 10 bp, synthetic construct
  source 1..10: mol_type = other DNA
cagtcggatt

SEQ ID NO: 255 - DNA, 10 bp, synthetic construct
  source 1..10: mol_type = other DNA
tgtgatcgcg

SEQ ID NO: 256 - DNA, 10 bp, synthetic construct
  source 1..10: mol_type = other DNA
acaatcacga

SEQ ID NO: 257 - DNA, 10 bp, synthetic construct
  source 1..10: mol_type = other DNA
tgtgactaca

SEQ ID NO: 258 - DNA, 10 bp, synthetic construct
  source 1..10: mol_type = other DNA
acacttgcgg

SEQ ID NO: 259 - DNA, 10 bp, synthetic construct
  source 1..10: mol_type = other DNA
ctgtggctat

SEQ ID NO: 260 - DNA, 10 bp, synthetic construct
  source 1..10: mol_type = other DNA
gacacaagtc

SEQ ID NO: 261 - DNA, 10 bp, synthetic construct
  source 1..10: mol_type = other DNA
tcatactgtc

SEQ ID NO: 262 - DNA, 10 bp, synthetic construct
  source 1..10: mol_type = other DNA
gatacggagg

SEQ ID NO: 263 - DNA, 10 bp, synthetic construct
  source 1..10: mol_type = other DNA
cggctaacca

SEQ ID NO: 264 - DNA, 10 bp, synthetic construct
  source 1..10: mol_type = other DNA
atcggtctat

SEQ ID NO: 265 - DNA, 10 bp, synthetic construct
  source 1..10: mol_type = other DNA
gcctcctgtt

SEQ ID NO: 266 - DNA, 10 bp, synthetic construct
  source 1..10: mol_type = other DNA
tataggacac

SEQ ID NO: 267 - DNA, 10 bp, synthetic construct
  source 1..10: mol_type = other DNA
agagaactga

SEQ ID NO: 268 - DNA, 10 bp, synthetic construct
  source 1..10: mol_type = other DNA
ctgcttgacg

SEQ ID NO: 269 - DNA, 10 bp, synthetic construct
  source 1..10: mol_type = other DNA
ccaactacca

SEQ ID NO: 270 - DNA, 10 bp, synthetic construct
  source 1..10: mol_type = other DNA
gtgcggcatt

SEQ ID NO: 271 - DNA, 10 bp, synthetic construct
  source 1..10: mol_type = other DNA
tgtgaaggag

SEQ ID NO: 272 - DNA, 10 bp, synthetic construct
  source 1..10: mol_type = other DNA
aacttcttgc

SEQ ID NO: 273 - DNA, 10 bp, synthetic construct
  source 1..10: mol_type = other DNA
tcgtggaatt

SEQ ID NO: 274 - DNA, 10 bp, synthetic construct
  source 1..10: mol_type = other DNA
aatacagcgc

SEQ ID NO: 275 - DNA, 10 bp, synthetic construct
  source 1..10: mol_type = other DNA
gtcctcctaa

SEQ ID NO: 276 - DNA, 10 bp, synthetic construct
  source 1..10: mol_type = other DNA
cgagattgcg

SEQ ID NO: 277 - DNA, 10 bp, synthetic construct
  source 1..10: mol_type = other DNA
ccgtagttgg

SEQ ID NO: 278 - DNA, 10 bp, synthetic construct
  source 1..10: mol_type = other DNA
ttagctcgtt

SEQ ID NO: 279 - DNA, 10 bp, synthetic construct
  source 1..10: mol_type = other DNA
gatcgcacaa

SEQ ID NO: 280 - DNA, 10 bp, synthetic construct
  source 1..10: mol_type = other DNA
agcatagacc

SEQ ID NO: 281 - DNA, 10 bp, synthetic construct
  source 1..10: mol_type = other DNA
agtcgccatt

SEQ ID NO: 282 - DNA, 10 bp, synthetic construct
  source 1..10: mol_type = other DNA
gagtaattgg

SEQ ID NO: 283 - DNA, 10 bp, synthetic construct
  source 1..10: mol_type = other DNA
ctagctagca

SEQ ID NO: 284 - DNA, 10 bp, synthetic construct
  source 1..10: mol_type = other DNA
tccatggcac

SEQ ID NO: 285 - DNA, 10 bp, synthetic construct
  source 1..10: mol_type = other DNA
tagcttccgg

SEQ ID NO: 286 - DNA, 10 bp, synthetic construct
  source 1..10: mol_type = other DNA
acttacagct

SEQ ID NO: 287 - DNA, 10 bp, synthetic construct
  source 1..10: mol_type = other DNA
cgcagatata

SEQ ID NO: 288 - DNA, 10 bp, synthetic construct
  source 1..10: mol_type = other DNA
gtagcggtac

SEQ ID NO: 289 - DNA, 10 bp, synthetic construct
  source 1..10: mol_type = other DNA
tctaatcgca

SEQ ID NO: 290 - DNA, 10 bp, synthetic construct
  source 1..10: mol_type = other DNA
aagctaacac

SEQ ID NO: 291 - DNA, 10 bp, synthetic construct
  source 1..10: mol_type = other DNA
cgagcgtagt

SEQ ID NO: 292 - DNA, 10 bp, synthetic construct
  source 1..10: mol_type = other DNA
gtctgcgttg

SEQ ID NO: 293 - DNA, 10 bp, synthetic construct
  source 1..10: mol_type = other DNA
tgaacaactc

SEQ ID NO: 294 - DNA, 10 bp, synthetic construct
  source 1..10: mol_type = other DNA
actcagtgct

SEQ ID NO: 295 - DNA, 10 bp, synthetic construct
  source 1..10: mol_type = other DNA
ctcgttcagg

SEQ ID NO: 296 - DNA, 10 bp, synthetic construct
  source 1..10: mol_type = other DNA
gagtgcgtaa

SEQ ID NO: 297 - DNA, 10 bp, synthetic construct
  source 1..10: mol_type = other DNA
cgcgaccatt

SEQ ID NO: 298 - DNA, 10 bp, synthetic construct
  source 1..10: mol_type = other DNA
aatactggca

SEQ ID NO: 299 - DNA, 10 bp, synthetic construct
  source 1..10: mol_type = other DNA
ttacggacgc

SEQ ID NO: 300 - DNA, 10 bp, synthetic construct
  source 1..10: mol_type = other DNA
gcgttattag

SEQ ID NO: 301 - DNA, 10 bp, synthetic construct
  source 1..10: mol_type = other DNA
tcagttcgat

SEQ ID NO: 302 - DNA, 10 bp, synthetic construct
  source 1..10: mol_type = other DNA
ctgagcaagc

SEQ ID NO: 303 - DNA, 10 bp, synthetic construct
  source 1..10: mol_type = other DNA
gactaagttg

SEQ ID NO: 304 - DNA, 10 bp, synthetic construct
  source 1..10: mol_type = other DNA
agtccgtcca

SEQ ID NO: 305 - DNA, 10 bp, synthetic construct
  source 1..10: mol_type = other DNA
gcggatattc

SEQ ID NO: 306 - DNA, 10 bp, synthetic construct
  source 1..10: mol_type = other DNA
ataccgcaag

SEQ ID NO: 307 - DNA, 10 bp, synthetic construct
  source 1..10: mol_type = other DNA
tgttgcgcgt

SEQ ID NO: 308 - DNA, 10 bp, synthetic construct
  source 1..10: mol_type = other DNA
cacatatgca

SEQ ID NO: 309 - DNA, 10 bp, synthetic construct
  source 1..10: mol_type = other DNA
agcgtgacct

SEQ ID NO: 310 - DNA, 10 bp, synthetic construct
  source 1..10: mol_type = other DNA
gtataacgac

SEQ ID NO: 311 - DNA, 10 bp, synthetic construct
  source 1..10: mol_type = other DNA
tagagctatg

SEQ ID NO: 312 - DNA, 10 bp, synthetic construct
  source 1..10: mol_type = other DNA
cctcctgtga

SEQ ID NO: 313 - DNA, 10 bp, synthetic construct
  source 1..10: mol_type = other DNA
ctcaaggctc

SEQ ID NO: 314 - DNA, 10 bp, synthetic construct
  source 1..10: mol_type = other DNA
gcaccttagg

SEQ ID NO: 315 - DNA, 10 bp, synthetic construct
  source 1..10: mol_type = other DNA
tgtgtactct

SEQ ID NO: 316 - DNA, 10 bp, synthetic construct
  source 1..10: mol_type = other DNA
aagtgcagaa

SEQ ID NO: 317 - DNA, 10 bp, synthetic construct
  source 1..10: mol_type = other DNA
tgcgacgata

SEQ ID NO: 318 - DNA, 10 bp, synthetic construct
  source 1..10: mol_type = other DNA
atgacgatcg

SEQ ID NO: 319 - DNA, 10 bp, synthetic construct
  source 1..10: mol_type = other DNA
cattgttcac

SEQ ID NO: 320 - DNA, 10 bp, synthetic construct
  source 1..10: mol_type = other DNA
gcactacggt

SEQ ID NO: 321 - DNA, 10 bp, synthetic construct
  source 1..10: mol_type = other DNA
gcaacctgag

SEQ ID NO: 322 - DNA, 10 bp, synthetic construct
  source 1..10: mol_type = other DNA
tagtagcact

SEQ ID NO: 323 - DNA, 10 bp, synthetic construct
  source 1..10: mol_type = other DNA
agccttattc

SEQ ID NO: 324 - DNA, 10 bp, synthetic construct
  source 1..10: mol_type = other DNA
cttggagcga

SEQ ID NO: 325 - DNA, 10 bp, synthetic construct
  source 1..10: mol_type = other DNA
agacgtgtta

SEQ ID NO: 326 - DNA, 10 bp, synthetic construct
  source 1..10: mol_type = other DNA
ccggtgagat

SEQ ID NO: 327 - DNA, 10 bp, synthetic construct
  source 1..10: mol_type = other DNA
gtctcacagc

SEQ ID NO: 328 - DNA, 10 bp, synthetic construct
  source 1..10: mol_type = other DNA
tataactccg

SEQ ID NO: 329 - DNA, 10 bp, synthetic construct
  source 1..10: mol_type = other DNA
gaagtccttg

SEQ ID NO: 330 - DNA, 10 bp, synthetic construct
  source 1..10: mol_type = other DNA
tccaatgcca

SEQ ID NO: 331 - DNA, 10 bp, synthetic construct
  source 1..10: mol_type = other DNA
atgtcgagac

SEQ ID NO: 332 - DNA, 10 bp, synthetic construct
  source 1..10: mol_type = other DNA
cgtcgatagt

SEQ ID NO: 333 - DNA, 10 bp, synthetic construct
  source 1..10: mol_type = other DNA
gattacaggt

SEQ ID NO: 334 - DNA, 10 bp, synthetic construct
  source 1..10: mol_type = other DNA
tccggagaac

SEQ ID NO: 335 - DNA, 10 bp, synthetic construct
  source 1..10: mol_type = other DNA
ataccgtcta

SEQ ID NO: 336 - DNA, 10 bp, synthetic construct
  source 1..10: mol_type = other DNA
cggattctcg

SEQ ID NO: 337 - DNA, 10 bp, synthetic construct
  source 1..10: mol_type = other DNA
aactgaccgt

SEQ ID NO: 338 - DNA, 10 bp, synthetic construct
  source 1..10: mol_type = other DNA
gtacttatcc

SEQ ID NO: 339 - DNA, 10 bp, synthetic construct
  source 1..10: mol_type = other DNA
cgtacgtaag

SEQ ID NO: 340 - DNA, 10 bp, synthetic construct
  source 1..10: mol_type = other DNA
tcggacggta

SEQ ID NO: 341 - DNA, 10 bp, synthetic construct
  source 1..10: mol_type = other DNA
aggacagagg

SEQ ID NO: 342 - DNA, 10 bp, synthetic construct
  source 1..10: mol_type = other DNA
tctgacacaa

SEQ ID NO: 343 - DNA, 10 bp, synthetic construct
  source 1..10: mol_type = other DNA
gaatgttgtc

SEQ ID NO: 344 - DNA, 10 bp, synthetic construct
  source 1..10: mol_type = other DNA
ctcctgctct

SEQ ID NO: 345 - DNA, 10 bp, synthetic construct
  source 1..10: mol_type = other DNA
agaagatgca

SEQ ID NO: 346 - DNA, 10 bp, synthetic construct
  source 1..10: mol_type = other DNA
gttccgcttc

SEQ ID NO: 347 - DNA, 10 bp, synthetic construct
  source 1..10: mol_type = other DNA
cactatgagt

SEQ ID NO: 348 - DNA, 10 bp, synthetic construct
  source 1..10: mol_type = other DNA
tcggtcacag

SEQ ID NO: 349 - DNA, 10 bp, synthetic construct
  source 1..10: mol_type = other DNA
gtcaggcatg

SEQ ID NO: 350 - DNA, 10 bp, synthetic construct
  source 1..10: mol_type = other DNA
tcagtcatgt

SEQ ID NO: 351 - DNA, 10 bp, synthetic construct
  source 1..10: mol_type = other DNA
aagcaatgac

SEQ ID NO: 352 - DNA, 10 bp, synthetic construct
  source 1..10: mol_type = other DNA
cgttctgcca

SEQ ID NO: 353 - DNA, 10 bp, synthetic construct
  source 1..10: mol_type = other DNA
gaagacgagt

SEQ ID NO: 354 - DNA, 10 bp, synthetic construct
  source 1..10: mol_type = other DNA
cttcttcctc

SEQ ID NO: 355 - DNA, 10 bp, synthetic construct
  source 1..10: mol_type = other DNA
aggagaagaa

SEQ ID NO: 356 - DNA, 10 bp, synthetic construct
  source 1..10: mol_type = other DNA
tcctcgttcg

SEQ ID NO: 357 - DNA, 10 bp, synthetic construct
  source 1..10: mol_type = other DNA
acatgcgtaa

SEQ ID NO: 358 - DNA, 10 bp, synthetic construct
  source 1..10: mol_type = other DNA
tgcaagtagc

SEQ ID NO: 359 - DNA, 10 bp, synthetic construct
  source 1..10: mol_type = other DNA
cttccaagct

SEQ ID NO: 360 - DNA, 10 bp, synthetic construct
  source 1..10: mol_type = other DNA
gaggttcctg

SEQ ID NO: 361 - DNA, 10 bp, synthetic construct
  source 1..10: mol_type = other DNA
cacgttgcag

SEQ ID NO: 362 - DNA, 10 bp, synthetic construct
  source 1..10: mol_type = other DNA
gtgaacaaga

SEQ ID NO: 363 - DNA, 10 bp, synthetic construct
  source 1..10: mol_type = other DNA
tgttggtgtc

SEQ ID NO: 364 - DNA, 10 bp, synthetic construct
  source 1..10: mol_type = other DNA
acaccactct

SEQ ID NO: 365 - DNA, 10 bp, synthetic construct
  source 1..10: mol_type = other DNA
agactcgcga

SEQ ID NO: 366 - DNA, 10 bp, synthetic construct
  source 1..10: mol_type = other DNA
tagtgaatcg

SEQ ID NO: 367 - DNA, 10 bp, synthetic construct
  source 1..10: mol_type = other DNA
cctgcttaat

SEQ ID NO: 368 - DNA, 10 bp, synthetic construct
  source 1..10: mol_type = other DNA
gtcaagcgtc

SEQ ID NO: 369 - DNA, 10 bp, synthetic construct
  source 1..10: mol_type = other DNA
cctataatgc

SEQ ID NO: 370 - DNA, 10 bp, synthetic construct
  source 1..10: mol_type = other DNA
agctaccaat

SEQ ID NO: 371 - DNA, 10 bp, synthetic construct
  source 1..10: mol_type = other DNA
ttacgtgccg

SEQ ID NO: 372 - DNA, 10 bp, synthetic construct
  source 1..10: mol_type = other DNA
gaggcgtgta

SEQ ID NO: 373 - DNA, 10 bp, synthetic construct
  source 1..10: mol_type = other DNA
gttgccatta

SEQ ID NO: 374 - DNA, 10 bp, synthetic construct
  source 1..10: mol_type = other DNA
accaattcgg

SEQ ID NO: 375 - DNA, 10 bp, synthetic construct
  source 1..10: mol_type = other DNA
cgatgagact

SEQ ID NO: 376 - DNA, 10 bp, synthetic construct
  source 1..10: mol_type = other DNA
tagctgcgac

SEQ ID NO: 377 - DNA, 10 bp, synthetic construct
  source 1..10: mol_type = other DNA
cgagttgaat

SEQ ID NO: 378 - DNA, 10 bp, synthetic construct
  source 1..10: mol_type = other DNA
gacaccttcc

SEQ ID NO: 379 - DNA, 10 bp, synthetic construct
  source 1..10: mol_type = other DNA
ttgcaacgta

SEQ ID NO: 380 - DNA, 10 bp, synthetic construct
  source 1..10: mol_type = other DNA
acttggacgg

SEQ ID NO: 381 - DNA, 10 bp, synthetic construct
  source 1..10: mol_type = other DNA
gtcatcgcgt

SEQ ID NO: 382 - DNA, 10 bp, synthetic construct
  source 1..10: mol_type = other DNA
taggaatgca

SEQ ID NO: 383 - DNA, 10 bp, synthetic construct
  source 1..10: mol_type = other DNA
ccttgtctac

SEQ ID NO: 384 - DNA, 10 bp, synthetic construct
  source 1..10: mol_type = other DNA
agaccgaatg

SEQ ID NO: 385 - DNA, 10 bp, synthetic construct
  source 1..10: mol_type = other DNA
ataggcgtta

SEQ ID NO: 386 - DNA, 10 bp, synthetic construct
  source 1..10: mol_type = other DNA
tgccatacgg

SEQ ID NO: 387 - DNA, 10 bp, synthetic construct
  source 1..10: mol_type = other DNA
cattcgtgcc

SEQ ID NO: 388 - DNA, 10 bp, synthetic construct
  source 1..10: mol_type = other DNA
gcgatacaat

SEQ ID NO: 389 - DNA, 10 bp, synthetic construct
  source 1..10: mol_type = other DNA
ttaacgcgac

SEQ ID NO: 390 - DNA, 10 bp, synthetic construct
  source 1..10: mol_type = other DNA
catgttgtca

SEQ ID NO: 391 - DNA, 10 bp, synthetic construct
  source 1..10: mol_type = other DNA
aggcgatctg

SEQ ID NO: 392 - DNA, 10 bp, synthetic construct
  source 1..10: mol_type = other DNA
gcctacaagt

SEQ ID NO: 393 - DNA, 10 bp, synthetic construct
  source 1..10: mol_type = other DNA
gaggttagta

SEQ ID NO: 394 - DNA, 10 bp, synthetic construct
  source 1..10: mol_type = other DNA
agttccgcct

SEQ ID NO: 395 - DNA, 10 bp, synthetic construct
  source 1..10: mol_type = other DNA
ccacagcaag

SEQ ID NO: 396 - DNA, 10 bp, synthetic construct
  source 1..10: mol_type = other DNA
ttcagattgc

SEQ ID NO: 397 - DNA, 10 bp, synthetic construct
  source 1..10: mol_type = other DNA
cggatacact

SEQ ID NO: 398 - DNA, 10 bp, synthetic construct
  source 1..10: mol_type = other DNA
gacgatagga

SEQ ID NO: 399 - DNA, 10 bp, synthetic construct
  source 1..10: mol_type = other DNA
tctcggtctg

SEQ ID NO: 400 - DNA, 10 bp, synthetic construct
  source 1..10: mol_type = other DNA
atatccgtac

SEQ ID NO: 401 - DNA, 10 bp, synthetic construct
  source 1..10: mol_type = other DNA
agatagcctt

SEQ ID NO: 402 - DNA, 10 bp, synthetic construct
  source 1..10: mol_type = other DNA
cttaccatgc

SEQ ID NO: 403 - DNA, 10 bp, synthetic construct
  source 1..10: mol_type = other DNA
tcggttggag

SEQ ID NO: 404 - DNA, 10 bp, synthetic construct
  source 1..10: mol_type = other DNA
gaccgataca

SEQ ID NO: 405 - DNA, 10 bp, synthetic construct
  source 1..10: mol_type = other DNA
aacaacctca

SEQ ID NO: 406 - DNA, 10 bp, synthetic construct
  source 1..10: mol_type = other DNA
cggctgtaac

SEQ ID NO: 407 - DNA, 10 bp, synthetic construct
  source 1..10: mol_type = other DNA
gtagcaggtg

SEQ ID NO: 408 - DNA, 10 bp, synthetic construct
  source 1..10: mol_type = other DNA
tcttgtacgt

SEQ ID NO: 409 - DNA, 10 bp, synthetic construct
  source 1..10: mol_type = other DNA
acgtgaacac

SEQ ID NO: 410 - DNA, 10 bp, synthetic construct
  source 1..10: mol_type = other DNA
cgcactggta

SEQ ID NO: 411 - DNA, 10 bp, synthetic construct
  source 1..10: mol_type = other DNA
gtagagctct

SEQ ID NO: 412 - DNA, 10 bp, synthetic construct
  source 1..10: mol_type = other DNA
tatctctagg

SEQ ID NO: 413 - DNA, 10 bp, synthetic construct
  source 1..10: mol_type = other DNA
aacaacttgg

SEQ ID NO: 414 - DNA, 10 bp, synthetic construct
  source 1..10: mol_type = other DNA
ccaccaccta

SEQ ID NO: 415 - DNA, 10 bp, synthetic construct
  source 1..10: mol_type = other DNA
gtggtgagac

SEQ ID NO: 416 - DNA, 10 bp, synthetic construct
  source 1..10: mol_type = other DNA
tgttgtgact

SEQ ID NO: 417 - DNA, 10 bp, synthetic construct
  source 1..10: mol_type = other DNA
ccattgaatg

SEQ ID NO: 418 - DNA, 10 bp, synthetic construct
  source 1..10: mol_type = other DNA
ttcaatcgac

SEQ ID NO: 419 - DNA, 10 bp, synthetic construct
  source 1..10: mol_type = other DNA
aggcgcttct

SEQ ID NO: 420 - DNA, 10 bp, synthetic construct
  source 1..10: mol_type = other DNA
gatgcagcga

SEQ ID NO: 421 - DNA, 10 bp, synthetic construct
  source 1..10: mol_type = other DNA
cacgatgtga

SEQ ID NO: 422 - DNA, 10 bp, synthetic construct
  source 1..10: mol_type = other DNA
tggtccaatt

SEQ ID NO: 423 - DNA, 10 bp, synthetic construct
  source 1..10: mol_type = other DNA
attaggtgcc

SEQ ID NO: 424 - DNA, 10 bp, synthetic construct
  source 1..10: mol_type = other DNA
gcactaccag

SEQ ID NO: 425 - DNA, 10 bp, synthetic construct
  source 1..10: mol_type = other DNA
aagtgatcca

SEQ ID NO: 426 - DNA, 10 bp, synthetic construct
  source 1..10: mol_type = other DNA
gtacatcttc

SEQ ID NO: 427 - DNA, 10 bp, synthetic construct
  source 1..10: mol_type = other DNA
tctgccggat

SEQ ID NO: 428 - DNA, 10 bp, synthetic construct
  source 1..10: mol_type = other DNA
cgcatgaagg

SEQ ID NO: 429 - DNA, 10 bp, synthetic construct
  source 1..10: mol_type = other DNA
actgagtaag

SEQ ID NO: 430 - DNA, 10 bp, synthetic construct
  source 1..10: mol_type = other DNA
ctcacagcga

SEQ ID NO: 431 - DNA, 10 bp, synthetic construct
  source 1..10: mol_type = other DNA
gagttcagct

SEQ ID NO: 432 - DNA, 10 bp, synthetic construct
  source 1..10: mol_type = other DNA
tgacgtcttc

SEQ ID NO: 433 - DNA, 10 bp, synthetic construct
  source 1..10: mol_type = other DNA
cgcctgttgt

SEQ ID NO: 434 - DNA, 10 bp, synthetic construct
  source 1..10: mol_type = other DNA
gagaatagca

SEQ ID NO: 435 - DNA, 10 bp, synthetic construct
  source 1..10: mol_type = other DNA
ttagcacatc

SEQ ID NO: 436 - DNA, 10 bp, synthetic construct
  source 1..10: mol_type = other DNA
acttgcgcag

SEQ ID NO: 437 - DNA, 10 bp, synthetic construct
  source 1..10: mol_type = other DNA
cgagaattca

SEQ ID NO: 438 - DNA, 10 bp, synthetic construct
  source 1..10: mol_type = other DNA
gcgttcgctt

SEQ ID NO: 439 - DNA, 10 bp, synthetic construct
  source 1..10: mol_type = other DNA
ttccgtaagc

SEQ ID NO: 440 - DNA, 10 bp, synthetic construct
  source 1..10: mol_type = other DNA
aatacgcgag

SEQ ID NO: 441 - DNA, 10 bp, synthetic construct
  source 1..10: mol_type = other DNA
cgtgattggt

SEQ ID NO: 442 - DNA, 10 bp, synthetic construct
  source 1..10: mol_type = other DNA
atcatgctac

SEQ ID NO: 443 - DNA, 10 bp, synthetic construct
  source 1..10: mol_type = other DNA
gcatccgatg

SEQ ID NO: 444 - DNA, 10 bp, synthetic construct
  source 1..10: mol_type = other DNA
tagcgaacca

SEQ ID NO: 445 - DNA, 10 bp, synthetic construct
  source 1..10: mol_type = other DNA
gatcctctcg

SEQ ID NO: 446 - DNA, 10 bp, synthetic construct
  source 1..10: mol_type = other DNA
ttgaaggaac

SEQ ID NO: 447 - DNA, 10 bp, synthetic construct
  source 1..10: mol_type = other DNA
ccattctgta

SEQ ID NO: 448 - DNA, 10 bp, synthetic construct
  source 1..10: mol_type = other DNA
agcggaacgt

SEQ ID NO: 449 - DNA, 10 bp, synthetic construct
  source 1..10: mol_type = other DNA
ttccacctgg

SEQ ID NO: 450 - DNA, 10 bp, synthetic construct
  source 1..10: mol_type = other DNA
gcatcttctt

SEQ ID NO: 451 - DNA, 10 bp, synthetic construct
  source 1..10: mol_type = other DNA
aatggagaac

SEQ ID NO: 452 - DNA, 10 bp, synthetic construct
  source 1..10: mol_type = other DNA
cggatgagca

SEQ ID NO: 453 - DNA, 10 bp, synthetic construct
  source 1..10: mol_type = other DNA
catgcctatg

SEQ ID NO: 454 - DNA, 10 bp, synthetic construct
  source 1..10: mol_type = other DNA
gcgtggacaa

SEQ ID NO: 455 - DNA, 10 bp, synthetic construct
  source 1..10: mol_type = other DNA
agcaatggct

SEQ ID NO: 456 - DNA, 10 bp, synthetic construct
  source 1..10: mol_type = other DNA
ttactactgc

SEQ ID NO: 457 - DNA, 10 bp, synthetic construct
  source 1..10: mol_type = other DNA
cggtaatatc

SEQ ID NO: 458 - DNA, 10 bp, synthetic construct
  source 1..10: mol_type = other DNA
acagccggaa

SEQ ID NO: 459 - DNA, 10 bp, synthetic construct
  source 1..10: mol_type = other DNA
gttctgctcg

SEQ ID NO: 460 - DNA, 10 bp, synthetic construct
  source 1..10: mol_type = other DNA
tacagtacgt

SEQ ID NO: 461 - DNA, 10 bp, synthetic construct
  source 1..10: mol_type = other DNA
cgtggagttg

SEQ ID NO: 462 - DNA, 10 bp, synthetic construct
  source 1..10: mol_type = other DNA
gtcttctcga

SEQ ID NO: 463 - DNA, 10 bp, synthetic construct
  source 1..10: mol_type = other DNA
tcacctcaat

SEQ ID NO: 464 - DNA, 10 bp, synthetic construct
  source 1..10: mol_type = other DNA
aagaagagcc

SEQ ID NO: 465 - DNA, 10 bp, synthetic construct
  source 1..10: mol_type = other DNA
aacgccgtca

SEQ ID NO: 466 - DNA, 10 bp, synthetic construct
  source 1..10: mol_type = other DNA
gcgctgaatc

SEQ ID NO: 467 - DNA, 10 bp, synthetic construct
  source 1..10: mol_type = other DNA
ctaagttggt

SEQ ID NO: 468 - DNA, 10 bp, synthetic construct
  source 1..10: mol_type = other DNA
tgttaaccag

SEQ ID NO: 469 - DNA, 10 bp, synthetic construct
  source 1..10: mol_type = other DNA
cgatacctag

SEQ ID NO: 470 - DNA, 10 bp, synthetic construct
  source 1..10: mol_type = other DNA
gctatgacct

SEQ ID NO: 471 - DNA, 10 bp, synthetic construct
  source 1..10: mol_type = other DNA
ttcgctggtc

SEQ ID NO: 472 - DNA, 10 bp, synthetic construct
  source 1..10: mol_type = other DNA
aagcgataga

SEQ ID NO: 473 - DNA, 10 bp, synthetic construct
  source 1..10: mol_type = other DNA
tctgatggaa

SEQ ID NO: 474 - DNA, 10 bp, synthetic construct
  source 1..10: mol_type = other DNA
agaacgtccg

SEQ ID NO: 475 - DNA, 10 bp, synthetic construct
  source 1..10: mol_type = other DNA
gtcttccatt

SEQ ID NO: 476 - DNA, 10 bp, synthetic construct
  source 1..10: mol_type = other DNA
cagcgaatgc

SEQ ID NO: 477 - DNA, 10 bp, synthetic construct
  source 1..10: mol_type = other DNA
cgagcactgt

SEQ ID NO: 478 - DNA, 10 bp, synthetic construct
  source 1..10: mol_type = other DNA
gctattgatc

SEQ ID NO: 479 - DNA, 10 bp, synthetic construct
  source 1..10: mol_type = other DNA
tagtacacag

SEQ ID NO: 480 - DNA, 10 bp, synthetic construct
  source 1..10: mol_type = other DNA
atccggtgca

SEQ ID NO: 481 - DNA, 10 bp, synthetic construct
  source 1..10: mol_type = other DNA
aacttggtgt

SEQ ID NO: 482 - DNA, 10 bp, synthetic construct
  source 1..10: mol_type = other DNA
gcacgaagtg

SEQ ID NO: 483 - DNA, 10 bp, synthetic construct
  source 1..10: mol_type = other DNA
cgtaactacc

SEQ ID NO: 484 - DNA, 10 bp, synthetic construct
  source 1..10: mol_type = other DNA
ttggctccaa

SEQ ID NO: 485 - DNA, 10 bp, synthetic construct
  source 1..10: mol_type = other DNA
gttcatcgag

SEQ ID NO: 486 - DNA, 10 bp, synthetic construct
  source 1..10: mol_type = other DNA
tacacatcgc

SEQ ID NO: 487 - DNA, 10 bp, synthetic construct
  source 1..10: mol_type = other DNA
acgtgcgact

SEQ ID NO: 488 - DNA, 10 bp, synthetic construct
  source 1..10: mol_type = other DNA
cgagtgatta

SEQ ID NO: 489 - DNA, 10 bp, synthetic construct
  source 1..10: mol_type = other DNA
cagctcctct

SEQ ID NO: 490 - DNA, 10 bp, synthetic construct
  source 1..10: mol_type = other DNA
gcagataagc

SEQ ID NO: 491 - DNA, 10 bp, synthetic construct
  source 1..10: mol_type = other DNA
tgttgatgag

SEQ ID NO: 492 - DNA, 10 bp, synthetic construct
  source 1..10: mol_type = other DNA
atcacggcta

SEQ ID NO: 493 - DNA, 10 bp, synthetic construct
  source 1..10: mol_type = other DNA
ttcagaacag

SEQ ID NO: 494 - DNA, 10 bp, synthetic construct
  source 1..10: mol_type = other DNA
caagccgtga

SEQ ID NO: 495 - DNA, 10 bp, synthetic construct
  source 1..10: mol_type = other DNA
aggttgtgtt

SEQ ID NO: 496 - DNA, 10 bp, synthetic construct
  source 1..10: mol_type = other DNA
gctcatcacc

SEQ ID NO: 497 - DNA, 10 bp, synthetic construct
  source 1..10: mol_type = other DNA
ccatactcag

SEQ ID NO: 498 - DNA, 10 bp, synthetic construct
  source 1..10: mol_type = other DNA
gtgagtattc

SEQ ID NO: 499 - DNA, 10 bp, synthetic construct
  source 1..10: mol_type = other DNA
tgtgcggaca

SEQ ID NO: 500 - DNA, 10 bp, synthetic construct
  source 1..10: mol_type = other DNA
aacctacggt

SEQ ID NO: 501 - DNA, 10 bp, synthetic construct
  source 1..10: mol_type = other DNA
tgaaccgaag

SEQ ID NO: 502 - DNA, 10 bp, synthetic construct
  source 1..10: mol_type = other DNA
acctggcgta

SEQ ID NO: 503 - DNA, 10 bp, synthetic construct
  source 1..10: mol_type = other DNA
cagctatcct

SEQ ID NO: 504 - DNA, 10 bp, synthetic construct
  source 1..10: mol_type = other DNA
gttgatatgc

SEQ ID NO: 505 - DNA, 10 bp, synthetic construct
  source 1..10: mol_type = other DNA
gcctcatgac

SEQ ID NO: 506 - DNA, 10 bp, synthetic construct
  source 1..10: mol_type = other DNA
atacacacct

SEQ ID NO: 507 - DNA, 10 bp, synthetic construct
  source 1..10: mol_type = other DNA
tatggtgatg

SEQ ID NO: 508 - DNA, 10 bp, synthetic construct
  source 1..10: mol_type = other DNA
cggatgctga

SEQ ID NO: 509 - DNA, 10 bp, synthetic construct
  source 1..10: mol_type = other DNA
ccgcttgttg

SEQ ID NO: 510 - DNA, 10 bp, synthetic construct
  source 1..10: mol_type = other DNA
ttctgctaca

SEQ ID NO: 511 - DNA, 10 bp, synthetic construct
  source 1..10: mol_type = other DNA
agaacaaggt

SEQ ID NO: 512 - DNA, 10 bp, synthetic construct
  source 1..10: mol_type = other DNA
gatgagccac

SEQ ID NO: 513 - DNA, 10 bp, synthetic construct
  source 1..10: mol_type = other DNA
acaagctcta

SEQ ID NO: 514 - DNA, 10 bp, synthetic construct
  source 1..10: mol_type = other DNA
ctgcaggaag

SEQ ID NO: 515 - DNA, 10 bp, synthetic construct
  source 1..10: mol_type = other DNA
tgttctagct

SEQ ID NO: 516 - DNA, 10 bp, synthetic construct
  source 1..10: mol_type = other DNA
gacgtactgc

SEQ ID NO: 517 - DNA, 10 bp, synthetic construct
  source 1..10: mol_type = other DNA
gtaaggtgtt

SEQ ID NO: 518 - DNA, 10 bp, synthetic construct
  source 1..10: mol_type = other DNA
aaggctaagc

SEQ ID NO: 519 - DNA, 10 bp, synthetic construct
  source 1..10: mol_type = other DNA
cgcttcctaa

SEQ ID NO: 520 - DNA, 10 bp, synthetic construct
  source 1..10: mol_type = other DNA
tctcaagccg

SEQ ID NO: 521 - DNA, 10 bp, synthetic construct
  source 1..10: mol_type = other DNA
gctctatccg

SEQ ID NO: 522 - DNA, 10 bp, synthetic construct
  source 1..10: mol_type = other DNA
tgagctagta

SEQ ID NO: 523 - DNA, 10 bp, synthetic construct
  source 1..10: mol_type = other DNA
ctgtaggagc

SEQ ID NO: 524 - DNA, 10 bp, synthetic construct
  source 1..10: mol_type = other DNA
aacagcctat

SEQ ID NO: 525 - DNA, 10 bp, synthetic construct
  source 1..10: mol_type = other DNA
gcgcatcgtt

SEQ ID NO: 526 - DNA, 10 bp, synthetic construct
  source 1..10: mol_type = other DNA
ttaagcatcc

SEQ ID NO: 527 - DNA, 10 bp, synthetic construct
  source 1..10: mol_type = other DNA
cgcgtatcag

SEQ ID NO: 528 - DNA, 10 bp, synthetic construct
  source 1..10: mol_type = other DNA
aattcggaga

SEQ ID NO: 529 - DNA, 10 bp, synthetic construct
  source 1..10: mol_type = other DNA
agcgaatgat

SEQ ID NO: 530 - DNA, 10 bp, synthetic construct
  source 1..10: mol_type = other DNA
catcgtcctg

SEQ ID NO: 531 - DNA, 10 bp, synthetic construct
  source 1..10: mol_type = other DNA
gcgttcaaga

SEQ ID NO: 532 - DNA, 10 bp, synthetic construct
  source 1..10: mol_type = other DNA
ttaacggtcc

SEQ ID NO: 533 - DNA, 10 bp, synthetic construct
  source 1..10: mol_type = other DNA
cggatatcaa

SEQ ID NO: 534 - DNA, 10 bp, synthetic construct
  source 1..10: mol_type = other DNA
gtaccgaagc

SEQ ID NO: 535 - DNA, 10 bp, synthetic construct
  source 1..10: mol_type = other DNA
tactaccgtt

SEQ ID NO: 536 - DNA, 10 bp, synthetic construct
  source 1..10: mol_type = other DNA
actggtgtcg

SEQ ID NO: 537 - DNA, 10 bp, synthetic construct
  source 1..10: mol_type = other DNA
cgtaattcag

SEQ ID NO: 538 - DNA, 10 bp, synthetic construct
  source 1..10: mol_type = other DNA
gaagcggtca

SEQ ID NO: 539 - DNA, 10 bp, synthetic construct
  source 1..10: mol_type = other DNA
ttccgaaggt

SEQ ID NO: 540 - DNA, 10 bp, synthetic construct
  source 1..10: mol_type = other DNA
acgttccatc

SEQ ID NO: 541 - DNA, 10 bp, synthetic construct
  source 1..10: mol_type = other DNA
taggaccgtc

SEQ ID NO: 542 - DNA, 10 bp, synthetic construct
  source 1..10: mol_type = other DNA
agtcctgtaa

SEQ ID NO: 543 - DNA, 10 bp, synthetic construct
  source 1..10: mol_type = other DNA
ctaaggaagg

SEQ ID NO: 544 - DNA, 10 bp, synthetic construct
  source 1..10: mol_type = other DNA
gccttatcct

SEQ ID NO: 545 - DNA, 10 bp, synthetic construct
  source 1..10: mol_type = other DNA
cttggtgacc

SEQ ID NO: 546 - DNA, 10 bp, synthetic construct
  source 1..10: mol_type = other DNA
agaacacttg

SEQ ID NO: 547 - DNA, 10 bp, synthetic construct
  source 1..10: mol_type = other DNA
gcgcacacaa

SEQ ID NO: 548 - DNA, 10 bp, synthetic construct
  source 1..10: mol_type = other DNA
tacttgtggt

SEQ ID NO: 549 - DNA, 10 bp, synthetic construct
  source 1..10: mol_type = other DNA
ttcaacaagc

SEQ ID NO: 550 - DNA, 10 bp, synthetic construct
  source 1..10: mol_type = other DNA
catgcacgct

SEQ ID NO: 551 - DNA, 10 bp, synthetic construct
  source 1..10: mol_type = other DNA
gcatgtgtta

SEQ ID NO: 552 - DNA, 10 bp, synthetic construct
  source 1..10: mol_type = other DNA
aggctgtcag

SEQ ID NO: 553 - DNA, 10 bp, synthetic construct
  source 1..10: mol_type = other DNA
ttgtacggct

SEQ ID NO: 554 - DNA, 10 bp, synthetic construct
  source 1..10: mol_type = other DNA
cgagttaagg

SEQ ID NO: 555 - DNA, 10 bp, synthetic construct
  source 1..10: mol_type = other DNA
gatccactta

SEQ ID NO: 556 - DNA, 10 bp, synthetic construct
  source 1..10: mol_type = other DNA
accaggtcac

SEQ ID NO: 557 - DNA, 10 bp, synthetic construct
  source 1..10: mol_type = other DNA
tccacggttc

SEQ ID NO: 558 - DNA, 10 bp, synthetic construct
  source 1..10: mol_type = other DNA
atattctcgg

SEQ ID NO: 559 - DNA, 10 bp, synthetic construct
  source 1..10: mol_type = other DNA
cggcgaagat

SEQ ID NO: 560 - DNA, 10 bp, synthetic construct
  source 1..10: mol_type = other DNA
gatgatcaca

SEQ ID NO: 561 - DNA, 10 bp, synthetic construct
  source 1..10: mol_type = other DNA
gacctactat

SEQ ID NO: 562 - DNA, 10 bp, synthetic construct
  source 1..10: mol_type = other DNA
tggaatacca

SEQ ID NO: 563 - DNA, 10 bp, synthetic construct
  source 1..10: mol_type = other DNA
attgccgagc

SEQ ID NO: 564 - DNA, 10 bp, synthetic construct
  source 1..10: mol_type = other DNA
ccatggtgtg

SEQ ID NO: 565 - DNA, 10 bp, synthetic construct
  source 1..10: mol_type = other DNA
agattgtgcg

SEQ ID NO: 566 - DNA, 10 bp, synthetic construct
  source 1..10: mol_type = other DNA
gatgaagtgc

SEQ ID NO: 567 - DNA, 10 bp, synthetic construct
  source 1..10: mol_type = other DNA
tcgactacat

SEQ ID NO: 568 - DNA, 10 bp, synthetic construct
  source 1..10: mol_type = other DNA
ctccgccata

SEQ ID NO: 569 - DNA, 10 bp, synthetic construct
  source 1..10: mol_type = other DNA
agttgccgga

SEQ ID NO: 570 - DNA, 10 bp, synthetic construct
  source 1..10: mol_type = other DNA
ccaacttaac

SEQ ID NO: 571 - DNA, 10 bp, synthetic construct
  source 1..10: mol_type = other DNA
taggtgatct

SEQ ID NO: 572 - DNA, 10 bp, synthetic construct
  source 1..10: mol_type = other DNA
gtccaagctg

SEQ ID NO: 573 - DNA, 10 bp, synthetic construct
  source 1..10: mol_type = other DNA
tccaagctcc

SEQ ID NO: 574 - DNA, 10 bp, synthetic construct
  source 1..10: mol_type = other DNA
aggttcggtg

SEQ ID NO: 575 - DNA, 10 bp, synthetic construct
  source 1..10: mol_type = other DNA
ctaggtaaga

SEQ ID NO: 576 - DNA, 10 bp, synthetic construct
  source 1..10: mol_type = other DNA
gatccatcat

SEQ ID NO: 577 - DNA, 10 bp, synthetic construct
  source 1..10: mol_type = other DNA
tcaagtcgcc

SEQ ID NO: 578 - DNA, 10 bp, synthetic construct
  source 1..10: mol_type = other DNA
gtgtacacta

SEQ ID NO: 579 - DNA, 10 bp, synthetic construct
  source 1..10: mol_type = other DNA
cgcgtagtgt

SEQ ID NO: 580 - DNA, 10 bp, synthetic construct
  source 1..10: mol_type = other DNA
aatccgtaag

SEQ ID NO: 581 - DNA, 10 bp, synthetic construct
  source 1..10: mol_type = other DNA
tgcagaatcc

SEQ ID NO: 582 - DNA, 10 bp, synthetic construct
  source 1..10: mol_type = other DNA
acttaggagt

SEQ ID NO: 583 - DNA, 10 bp, synthetic construct
  source 1..10: mol_type = other DNA
ctggtctcaa

SEQ ID NO: 584 - DNA, 10 bp, synthetic construct
  source 1..10: mol_type = other DNA
gaacctcgtg

SEQ ID NO: 585 - DNA, 10 bp, synthetic construct
  source 1..10: mol_type = other DNA
tggctacata

SEQ ID NO: 586 - DNA, 10 bp, synthetic construct
  source 1..10: mol_type = other DNA
ccatgtagag

SEQ ID NO: 587 - DNA, 10 bp, synthetic construct
  source 1..10: mol_type = other DNA
gttgacgtcc

SEQ ID NO: 588 - DNA, 10 bp, synthetic construct
  source 1..10: mol_type = other DNA
aacacgtcgt

SEQ ID NO: 589 - DNA, 10 bp, synthetic construct
  source 1..10: mol_type = other DNA
taaccacctc

SEQ ID NO: 590 - DNA, 10 bp, synthetic construct
  source 1..10: mol_type = other DNA
acgtgcttat

SEQ ID NO: 591 - DNA, 10 bp, synthetic construct
  source 1..10: mol_type = other DNA
gtcgtggaga

SEQ ID NO: 592 - DNA, 10 bp, synthetic construct
  source 1..10: mol_type = other DNA
cgtaatagcg

SEQ ID NO: 593 - DNA, 10 bp, synthetic construct
  source 1..10: mol_type = other DNA
aaccgtggta

SEQ ID NO: 594 - DNA, 10 bp, synthetic construct
  source 1..10: mol_type = other DNA
gttgtactac

SEQ ID NO: 595 - DNA, 10 bp, synthetic construct
  source 1..10: mol_type = other DNA
tcatcgaagg

SEQ ID NO: 596 - DNA, 10 bp, synthetic construct
  source 1..10: mol_type = other DNA
cggaactcct

SEQ ID NO: 597 - DNA, 10 bp, synthetic construct
  source 1..10: mol_type = other DNA
agtacaactg

SEQ ID NO: 598 - DNA, 10 bp, synthetic construct
  source 1..10: mol_type = other DNA
caagtctgca

SEQ ID NO: 599 - DNA, 10 bp, synthetic construct
  source 1..10: mol_type = other DNA
gtgcagctac

SEQ ID NO: 600 - DNA, 10 bp, synthetic construct
  source 1..10: mol_type = other DNA
tcctgtgagt

SEQ ID NO: 601 - DNA, 10 bp, synthetic construct
  source 1..10: mol_type = other DNA
cactaatcag

SEQ ID NO: 602 - DNA, 10 bp, synthetic construct
  source 1..10: mol_type = other DNA
actagcggct

SEQ ID NO: 603 - DNA, 10 bp, synthetic construct
  source 1..10: mol_type = other DNA
gtagttcatc

SEQ ID NO: 604 - DNA, 10 bp, synthetic construct
  source 1..10: mol_type = other DNA
tggccgatga

SEQ ID NO: 605 - DNA, 10 bp, synthetic construct
  source 1..10: mol_type = other DNA
accatccgaa

SEQ ID NO: 606 - DNA, 10 bp, synthetic construct
  source 1..10: mol_type = other DNA
cattcgattc

SEQ ID NO: 607 - DNA, 10 bp, synthetic construct
  source 1..10: mol_type = other DNA
gtaggatagg

SEQ ID NO: 608 - DNA, 10 bp, synthetic construct
  source 1..10: mol_type = other DNA
tggcatgcct

SEQ ID NO: 609 - DNA, 10 bp, synthetic construct
  source 1..10: mol_type = other DNA
cgtagatcgc

SEQ ID NO: 610 - DNA, 10 bp, synthetic construct
  source 1..10: mol_type = other DNA
taagcgcgtg

SEQ ID NO: 611 - DNA, 10 bp, synthetic construct
  source 1..10: mol_type = other DNA
gtgcttgtat

SEQ ID NO: 612 - DNA, 10 bp, synthetic construct
  source 1..10: mol_type = other DNA
acctacaaca

SEQ ID NO: 613 - DNA, 10 bp, synthetic construct
  source 1..10: mol_type = other DNA
agtcatgaca

SEQ ID NO: 614 - DNA, 10 bp, synthetic construct
  source 1..10: mol_type = other DNA
ttgagcctgg

SEQ ID NO: 615 - DNA, 10 bp, synthetic construct
  source 1..10: mol_type = other DNA
caagtatctc

SEQ ID NO: 616 - DNA, 10 bp, synthetic construct
  source 1..10: mol_type = other DNA
gcctcgagat

SEQ ID NO: 617 - DNA, 10 bp, synthetic construct
  source 1..10: mol_type = other DNA
cactcgtagc

SEQ ID NO: 618 - DNA, 10 bp, synthetic construct
  source 1..10: mol_type = other DNA
gctggtacct

SEQ ID NO: 619 - DNA, 10 bp, synthetic construct
  source 1..10: mol_type = other DNA
aggatcctaa

SEQ ID NO: 620 - DNA, 10 bp, synthetic construct
  source 1..10: mol_type = other DNA
ttacaaggtg

SEQ ID NO: 621 - DNA, 10 bp, synthetic construct
  source 1..10: mol_type = other DNA
aatgaggcta

SEQ ID NO: 622 - DNA, 10 bp, synthetic construct
  source 1..10: mol_type = other DNA
gtaagtcgcg

SEQ ID NO: 623 - DNA, 10 bp, synthetic construct
  source 1..10: mol_type = other DNA
tgctcctaac

SEQ ID NO: 624 - DNA, 10 bp, synthetic construct
  source 1..10: mol_type = other DNA
ccgctaatgt

SEQ ID NO: 625 - DNA, 10 bp, synthetic construct
  source 1..10: mol_type = other DNA
agttagtcac

SEQ ID NO: 626 - DNA, 10 bp, synthetic construct
  source 1..10: mol_type = other DNA
ctcgttgtcg

SEQ ID NO: 627 - DNA, 10 bp, synthetic construct
  source 1..10: mol_type = other DNA
gcgagccata

SEQ ID NO: 628 - DNA, 10 bp, synthetic construct
  source 1..10: mol_type = other DNA
taaccaaggt

SEQ ID NO: 629 - DNA, 10 bp, synthetic construct
  source 1..10: mol_type = other DNA
ccagatgtgc

SEQ ID NO: 630 - DNA, 10 bp, synthetic construct
  source 1..10: mol_type = other DNA
gattcgtgag

SEQ ID NO: 631 - DNA, 10 bp, synthetic construct
  source 1..10: mol_type = other DNA
ttgctcacca

SEQ ID NO: 632 - DNA, 10 bp, synthetic construct
  source 1..10: mol_type = other DNA
agcagacatt

SEQ ID NO: 633 - DNA, 10 bp, synthetic construct
  source 1..10: mol_type = other DNA
tagcggagtg

SEQ ID NO: 634 - DNA, 10 bp, synthetic construct
  source 1..10: mol_type = other DNA
agcaactaga

SEQ ID NO: 635 - DNA, 10 bp, synthetic construct
  source 1..10: mol_type = other DNA
ctagttctct

SEQ ID NO: 636 - DNA, 10 bp, synthetic construct
  source 1..10: mol_type = other DNA
gcttcagcac

SEQ ID NO: 637 - DNA, 10 bp, synthetic construct
  source 1..10: mol_type = other DNA
cataatccgg

SEQ ID NO: 638 - DNA, 10 bp, synthetic construct
  source 1..10: mol_type = other DNA
gtccgctgat

SEQ ID NO: 639 - DNA, 10 bp, synthetic construct
  source 1..10: mol_type = other DNA
agatcagtcc

SEQ ID NO: 640 - DNA, 10 bp, synthetic construct
  source 1..10: mol_type = other DNA
tcggtgaata

SEQ ID NO: 641 - DNA, 10 bp, synthetic construct
  source 1..10: mol_type = other DNA
caagcctggt

SEQ ID NO: 642 - DNA, 10 bp, synthetic construct
  source 1..10: mol_type = other DNA
gtccttgcca

SEQ ID NO: 643 - DNA, 10 bp, synthetic construct
  source 1..10: mol_type = other DNA
tctaggattg

SEQ ID NO: 644 - DNA, 10 bp, synthetic construct
  source 1..10: mol_type = other DNA
aggtaacaac

SEQ ID NO: 645 - DNA, 10 bp, synthetic construct
  source 1..10: mol_type = other DNA
ctggaggtct

SEQ ID NO: 646 - DNA, 10 bp, synthetic construct
  source 1..10: mol_type = other DNA
agctctacaa

SEQ ID NO: 647 - DNA, 10 bp, synthetic construct
  source 1..10: mol_type = other DNA
gatatatggc

SEQ ID NO: 648 - DNA, 10 bp, synthetic construct
  source 1..10: mol_type = other DNA
tcacgccatg

SEQ ID NO: 649 - DNA, 10 bp, synthetic construct
  source 1..10: mol_type = other DNA
tggatcggtc

SEQ ID NO: 650 - DNA, 10 bp, synthetic construct
  source 1..10: mol_type = other DNA
atatagccgg

SEQ ID NO: 651 - DNA, 10 bp, synthetic construct
  source 1..10: mol_type = other DNA
cctccattat

SEQ ID NO: 652 - DNA, 10 bp, synthetic construct
  source 1..10: mol_type = other DNA
gacggtaaca

SEQ ID NO: 653 - DNA, 10 bp, synthetic construct
  source 1..10: mol_type = other DNA
caattcctca

SEQ ID NO: 654 - DNA, 10 bp, synthetic construct
  source 1..10: mol_type = other DNA
gcgagaagag

SEQ ID NO: 655 - DNA, 10 bp, synthetic construct
  source 1..10: mol_type = other DNA
ttccagtagt

SEQ ID NO: 656 - DNA, 10 bp, synthetic construct
  source 1..10: mol_type = other DNA
agtgctgctc

SEQ ID NO: 657 - DNA, 10 bp, synthetic construct
  source 1..10: mol_type = other DNA
taccgcacat

SEQ ID NO: 658 - DNA, 10 bp, synthetic construct
  source 1..10: mol_type = other DNA
gcagtacacg

SEQ ID NO: 659 - DNA, 10 bp, synthetic construct
  source 1..10: mol_type = other DNA
cgtacggtta

SEQ ID NO: 660 - DNA, 10 bp, synthetic construct
  source 1..10: mol_type = other DNA
atgtattggc

SEQ ID NO: 661 - DNA, 10 bp, synthetic construct
  source 1..10: mol_type = other DNA
ctgccttagt

SEQ ID NO: 662 - DNA, 10 bp, synthetic construct
  source 1..10: mol_type = other DNA
gatgtggtac

SEQ ID NO: 663 - DNA, 10 bp, synthetic construct
  source 1..10: mol_type = other DNA
tcctacagcg

SEQ ID NO: 664 - DNA, 10 bp, synthetic construct
  source 1..10: mol_type = other DNA
agaagaccta

SEQ ID NO: 665 - DNA, 10 bp, synthetic construct
  source 1..10: mol_type = other DNA
actggtgcaa

SEQ ID NO: 666 - DNA, 10 bp, synthetic construct
  source 1..10: mol_type = other DNA
cgccagaatc

SEQ ID NO: 667 - DNA, 10 bp, synthetic construct
  source 1..10: mol_type = other DNA
gtaatactcg

SEQ ID NO: 668 - DNA, 10 bp, synthetic construct
  source 1..10: mol_type = other DNA
tagtcctggt

SEQ ID NO: 669 - DNA, 10 bp, synthetic construct
  source 1..10: mol_type = other DNA
agtctaagga

SEQ ID NO: 670 - DNA, 10 bp, synthetic construct
  source 1..10: mol_type = other DNA
gaggacgatc

SEQ ID NO: 671 - DNA, 10 bp, synthetic construct
  source 1..10: mol_type = other DNA
ccatgttcat

SEQ ID NO: 672 - DNA, 10 bp, synthetic construct
  source 1..10: mol_type = other DNA
ttcacgctcg

SEQ ID NO: 673 - DNA, 10 bp, synthetic construct
  source 1..10: mol_type = other DNA
gatctagcac

SEQ ID NO: 674 - DNA, 10 bp, synthetic construct
  source 1..10: mol_type = other DNA
ctgtcgatgg

SEQ ID NO: 675 - DNA, 10 bp, synthetic construct
  source 1..10: mol_type = other DNA
tccagttaca

SEQ ID NO: 676 - DNA, 10 bp, synthetic construct
  source 1..10: mol_type = other DNA
agagaccgtt

SEQ ID NO: 677 - DNA, 10 bp, synthetic construct
  source 1..10: mol_type = other DNA
gtacaattcg

SEQ ID NO: 678 - DNA, 10 bp, synthetic construct
  source 1..10: mol_type = other DNA
cattccgcaa

SEQ ID NO: 679 - DNA, 10 bp, synthetic construct
  source 1..10: mol_type = other DNA
tggatgcagc

SEQ ID NO: 680 - DNA, 10 bp, synthetic construct
  source 1..10: mol_type = other DNA
accggtagtt

SEQ ID NO: 681 - DNA, 10 bp, synthetic construct
  source 1..10: mol_type = other DNA
atgcgttccg

SEQ ID NO: 682 - DNA, 10 bp, synthetic construct
  source 1..10: mol_type = other DNA
ccaacactac

SEQ ID NO: 683 - DNA, 10 bp, synthetic construct
  source 1..10: mol_type = other DNA
gacgtcgata

SEQ ID NO: 684 - DNA, 10 bp, synthetic construct
  source 1..10: mol_type = other DNA
tgttagaggt

SEQ ID NO: 685 - DNA, 10 bp, synthetic construct
  source 1..10: mol_type = other DNA
cctctcacgt

SEQ ID NO: 686 - DNA, 10 bp, synthetic construct
  source 1..10: mol_type = other DNA
gtctcagtcg

SEQ ID NO: 687 - DNA, 10 bp, synthetic construct
  source 1..10: mol_type = other DNA
taggagcata

SEQ ID NO: 688 - DNA, 10 bp, synthetic construct
  source 1..10: mol_type = other DNA
agaagttgac

SEQ ID NO: 689 - DNA, 10 bp, synthetic construct
  source 1..10: mol_type = other DNA
tggttcgtcg

SEQ ID NO: 690 - DNA, 10 bp, synthetic construct
  source 1..10: mol_type = other DNA
accgcacgtt

SEQ ID NO: 691 - DNA, 10 bp, synthetic construct
  source 1..10: mol_type = other DNA
cttaagtcac

SEQ ID NO: 692 - DNA, 10 bp, synthetic construct
  source 1..10: mol_type = other DNA
gaacgtaaga

SEQ ID NO: 693 - DNA, 10 bp, synthetic construct
  source 1..10: mol_type = other DNA
ctccgcaagt

SEQ ID NO: 694 - DNA, 10 bp, synthetic construct
  source 1..10: mol_type = other DNA
gatgttgcaa

SEQ ID NO: 695 - DNA, 10 bp, synthetic construct
  source 1..10: mol_type = other DNA
tgataatgcg

SEQ ID NO: 696 - DNA, 10 bp, synthetic construct
  source 1..10: mol_type = other DNA
acgacgcttc

SEQ ID NO: 697 - DNA, 10 bp, synthetic construct
  source 1..10: mol_type = other DNA
gcacagagac

SEQ ID NO: 698 - DNA, 10 bp, synthetic construct
  source 1..10: mol_type = other DNA
cttgcactca

SEQ ID NO: 699 - DNA, 10 bp, synthetic construct
  source 1..10: mol_type = other DNA
tacttcgagg

SEQ ID NO: 700 - DNA, 10 bp, synthetic construct
  source 1..10: mol_type = other DNA
aggagttctt

SEQ ID NO: 701 - DNA, 10 bp, synthetic construct
  source 1..10: mol_type = other DNA
cggacgttaa

SEQ ID NO: 702 - DNA, 10 bp, synthetic construct
  source 1..10: mol_type = other DNA
actcaacggc

SEQ ID NO: 703 - DNA, 10 bp, synthetic construct
  source 1..10: mol_type = other DNA
gtatgcaatg

SEQ ID NO: 704 - DNA, 10 bp, synthetic construct
  source 1..10: mol_type = other DNA
tacgttgcct

SEQ ID NO: 705 - DNA, 10 bp, synthetic construct
  source 1..10: mol_type = other DNA
actggctaac

SEQ ID NO: 706 - DNA, 10 bp, synthetic construct
  source 1..10: mol_type = other DNA
ctactgatgt

SEQ ID NO: 707 - DNA, 10 bp, synthetic construct
  source 1..10: mol_type = other DNA
gactatggca

SEQ ID NO: 708 - DNA, 10 bp, synthetic construct
  source 1..10: mol_type = other DNA
tggacacctg

SEQ ID NO: 709 - DNA, 10 bp, synthetic construct
  source 1..10: mol_type = other DNA
tctcaccagg

SEQ ID NO: 710 - DNA, 10 bp, synthetic construct
  source 1..10: mol_type = other DNA
aacgtaggct

SEQ ID NO: 711 - DNA, 10 bp, synthetic construct
  source 1..10: mol_type = other DNA
cgatcttctc

SEQ ID NO: 712 - DNA, 10 bp, synthetic construct
  source 1..10: mol_type = other DNA
gtgaggataa

SEQ ID NO: 713 - DNA, 10 bp, synthetic construct
  source 1..10: mol_type = other DNA
gcacttcatg

SEQ ID NO: 714 - DNA, 10 bp, synthetic construct
  source 1..10: mol_type = other DNA
ctggacaggt

SEQ ID NO: 715 - DNA, 10 bp, synthetic construct
  source 1..10: mol_type = other DNA
tgttggtcca

SEQ ID NO: 716 - DNA, 10 bp, synthetic construct
  source 1..10: mol_type = other DNA
aacacagtac

SEQ ID NO: 717 - DNA, 10 bp, synthetic construct
  source 1..10: mol_type = other DNA
ttcaaggaga

SEQ ID NO: 718 - DNA, 10 bp, synthetic construct
  source 1..10: mol_type = other DNA
ccgtctagtg

SEQ ID NO: 719 - DNA, 10 bp, synthetic construct
  source 1..10: mol_type = other DNA
agaggatcct

SEQ ID NO: 720 - DNA, 10 bp, synthetic construct
  source 1..10: mol_type = other DNA
gatctcctac

SEQ ID NO: 721 - DNA, 10 bp, synthetic construct
  source 1..10: mol_type = other DNA
accatgagac

SEQ ID NO: 722 - DNA, 10 bp, synthetic construct
  source 1..10: mol_type = other DNA
tgttcatctg

SEQ ID NO: 723 - DNA, 10 bp, synthetic construct
  source 1..10: mol_type = other DNA
ctagacctgt

SEQ ID NO: 724 - DNA, 10 bp, synthetic construct
  source 1..10: mol_type = other DNA
gagcgtgaca

SEQ ID NO: 725 - DNA, 10 bp, synthetic construct
  source 1..10: mol_type = other DNA
tgctggatct

SEQ ID NO: 726 - DNA, 10 bp, synthetic construct
  source 1..10: mol_type = other DNA
aaggcacaac

SEQ ID NO: 727 - DNA, 10 bp, synthetic construct
  source 1..10: mol_type = other DNA
ctaatctgga

SEQ ID NO: 728 - DNA, 10 bp, synthetic construct
  source 1..10: mol_type = other DNA
gctcatgctg

SEQ ID NO: 729 - DNA, 10 bp, synthetic construct
  source 1..10: mol_type = other DNA
gatcctcaac

SEQ ID NO: 730 - DNA, 10 bp, synthetic construct
  source 1..10: mol_type = other DNA
tgcagcagca

SEQ ID NO: 731 - DNA, 10 bp, synthetic construct
  source 1..10: mol_type = other DNA
ctagtgttgg

SEQ ID NO: 732 - DNA, 10 bp, synthetic construct
  source 1..10: mol_type = other DNA
acgtaagctt

SEQ ID NO: 733 - DNA, 10 bp, synthetic construct
  source 1..10: mol_type = other DNA
agtggatcaa

SEQ ID NO: 734 - DNA, 10 bp, synthetic construct
  source 1..10: mol_type = other DNA
tacatcaacg

SEQ ID NO: 735 - DNA, 10 bp, synthetic construct
  source 1..10: mol_type = other DNA
ctaccgcgtt

SEQ ID NO: 736 - DNA, 10 bp, synthetic construct
  source 1..10: mol_type = other DNA
gcgtatgtgc

SEQ ID NO: 737 - DNA, 10 bp, synthetic construct
  source 1..10: mol_type = other DNA
acactagcct

SEQ ID NO: 738 - DNA, 10 bp, synthetic construct
  source 1..10: mol_type = other DNA
tgtgagcgaa

SEQ ID NO: 739 - DNA, 10 bp, synthetic construct
  source 1..10: mol_type = other DNA
cactccatgc

SEQ ID NO: 740 - DNA, 10 bp, synthetic construct
  source 1..10: mol_type = other DNA
gtgagttatg

SEQ ID NO: 741 - DNA, 10 bp, synthetic construct
  source 1..10: mol_type = other DNA
tactcggaag

SEQ ID NO: 742 - DNA, 10 bp, synthetic construct
  source 1..10: mol_type = other DNA
attggcctgc

SEQ ID NO: 743 - DNA, 10 bp, synthetic construct
  source 1..10: mol_type = other DNA
cggcattgtt

SEQ ID NO: 744 - DNA, 10 bp, synthetic construct
  source 1..10: mol_type = other DNA
gcaataacca

SEQ ID NO: 745 - DNA, 10 bp, synthetic construct
  source 1..10: mol_type = other DNA
gtagcgagtt

SEQ ID NO: 746 - DNA, 10 bp, synthetic construct
  source 1..10: mol_type = other DNA
aaccgttcga

SEQ ID NO: 747 - DNA, 10 bp, synthetic construct
  source 1..10: mol_type = other DNA
tctatcgtac

SEQ ID NO: 748 - DNA, 10 bp, synthetic construct
  source 1..10: mol_type = other DNA
cggtaacacg

SEQ ID NO: 749 - DNA, 10 bp, synthetic construct
  source 1..10: mol_type = other DNA
gcaacttgtc

SEQ ID NO: 750 - DNA, 10 bp, synthetic construct
  source 1..10: mol_type = other DNA
atggagacgg

SEQ ID NO: 751 - DNA, 10 bp, synthetic construct
  source 1..10: mol_type = other DNA
cgtctagtaa

SEQ ID NO: 752 - DNA, 10 bp, synthetic construct
  source 1..10: mol_type = other DNA
tactgccact

SEQ ID NO: 753 - DNA, 10 bp, synthetic construct
  source 1..10: mol_type = other DNA
gatcaacaag

SEQ ID NO: 754 - DNA, 10 bp, synthetic construct
  source 1..10: mol_type = other DNA
acattcttcc

SEQ ID NO: 755 - DNA, 10 bp, synthetic construct
  source 1..10: mol_type = other DNA
ttggctgcgt

SEQ ID NO: 756 - DNA, 10 bp, synthetic construct
  source 1..10: mol_type = other DNA
cgcaggagta

SEQ ID NO: 757 - DNA, 10 bp, synthetic construct
  source 1..10: mol_type = other DNA
cgtgtatgtc

SEQ ID NO: 758 - DNA, 10 bp, synthetic construct
  source 1..10: mol_type = other DNA
gtctgtcaag

SEQ ID NO: 759 - DNA, 10 bp, synthetic construct
  source 1..10: mol_type = other DNA
tcaaccgcca

SEQ ID NO: 760 - DNA, 10 bp, synthetic construct
  source 1..10: mol_type = other DNA
aagcagatgt

SEQ ID NO: 761 - DNA, 10 bp, synthetic construct
  source 1..10: mol_type = other DNA
gacaagtggt

SEQ ID NO: 762 - DNA, 10 bp, synthetic construct
  source 1..10: mol_type = other DNA
ctggtacaca

SEQ ID NO: 763 - DNA, 10 bp, synthetic construct
  source 1..10: mol_type = other DNA
tgacctgctg

SEQ ID NO: 764 - DNA, 10 bp, synthetic construct
  source 1..10: mol_type = other DNA
acttgcatac

SEQ ID NO: 765 - DNA, 10 bp, synthetic construct
  source 1..10: mol_type = other DNA
agtgaggtca

SEQ ID NO: 766 - DNA, 10 bp, synthetic construct
  source 1..10: mol_type = other DNA
ctactaaggc

SEQ ID NO: 767 - DNA, 10 bp, synthetic construct
  source 1..10: mol_type = other DNA
gagtcctatt

SEQ ID NO: 768 - DNA, 10 bp, synthetic construct
  source 1..10: mol_type = other DNA
tccagtccag

SEQ ID NO: 769 - DNA, 31 bp, synthetic construct
  source 1..31: mol_type = other DNA
  modified_base 1: mod_base = OTHER; note = phosphorylation
agtcggaggccaagcggtcttaggaagacaa

SEQ ID NO: 770 - DNA, 40 bp, synthetic construct
  source 1..40: mol_type = other DNA
  modified_base 39^40: mod_base = OTHER; note = thio
ttgtcttcctaacaggaacgacatggctacgatccgactt

SEQ ID NO: 771 - DNA, 24 bp, synthetic construct
  source 1..24: mol_type = other DNA
ttgtcttcctaagaccgcttggcc

SEQ ID NO: 772 - DNA, 21 bp, synthetic construct
  source 1..21: mol_type = other DNA
cacagaacgacatggctacga

SEQ ID NO: 773 - DNA, 20 bp, synthetic construct
  source 1..20: mol_type = other DNA
ctctcagtacgtcagcagtt

SEQ ID NO: 774 - DNA, 34 bp, synthetic construct
  source 1..34: mol_type = other DNA
caactccttggctcacagaacgacatggctacga

SEQ ID NO: 775 - DNA, 18 bp, synthetic construct
  source 1..18: mol_type = other DNA
gcatggcgaccttatcag

SEQ ID NO: 776 - DNA, 10 bp, synthetic construct
  source 1..10: mol_type = other DNA
tcacattgct

SEQ ID NO: 777 - DNA, 10 bp, synthetic construct
  source 1..10: mol_type = other DNA
aatggcgctc

SEQ ID NO: 778 - DNA, 10 bp, synthetic construct
  source 1..10: mol_type = other DNA
gtctcaatga

SEQ ID NO: 779 - DNA, 10 bp, synthetic construct
  source 1..10: mol_type = other DNA
cggatgcaag

SEQ ID NO: 780 - DNA, 10 bp, synthetic construct
  source 1..10: mol_type = other DNA
tcgcttaagc

SEQ ID NO: 781 - DNA, 10 bp, synthetic construct
  source 1..10: mol_type = other DNA
cgaggcttag

SEQ ID NO: 782 - DNA, 10 bp, synthetic construct
  source 1..10: mol_type = other DNA
gtctaaggct

SEQ ID NO: 783 - DNA, 10 bp, synthetic construct
  source 1..10: mol_type = other DNA
aatacgccta

SEQ ID NO: 784 - DNA, 10 bp, synthetic construct
  source 1..10: mol_type = other DNA
aagcctattg

SEQ ID NO: 785 - DNA, 10 bp, synthetic construct
  source 1..10: mol_type = other DNA
cgctactgca

SEQ ID NO: 786 - DNA, 10 bp, synthetic construct
  source 1..10: mol_type = other DNA
tcaagagcat

SEQ ID NO: 787 - DNA, 10 bp, synthetic construct
  source 1..10: mol_type = other DNA
gttgtgcagc

SEQ ID NO: 788 - DNA, 10 bp, synthetic construct
  source 1..10: mol_type = other DNA
agacaggaat

SEQ ID NO: 789 - DNA, 10 bp, synthetic construct
  source 1..10: mol_type = other DNA
ccttgccgta

SEQ ID NO: 790 - DNA, 10 bp, synthetic construct
  source 1..10: mol_type = other DNA
gtcattacgg

SEQ ID NO: 791 - DNA, 10 bp, synthetic construct
  source 1..10: mol_type = other DNA
taggcattcc

SEQ ID NO: 792 - DNA, 10 bp, synthetic construct
  source 1..10: mol_type = other DNA
catatcatcg

SEQ ID NO: 793 - DNA, 10 bp, synthetic construct
  source 1..10: mol_type = other DNA
gcacaacaat

SEQ ID NO: 794 - DNA, 10 bp, synthetic construct
  source 1..10: mol_type = other DNA
ttgtcgtggc

SEQ ID NO: 795 - DNA, 10 bp, synthetic construct
  source 1..10: mol_type = other DNA
agcggtgcta

SEQ ID NO: 796 - DNA, 10 bp, synthetic construct
  source 1..10: mol_type = other DNA
agccagtagg

SEQ ID NO: 797 - DNA, 10 bp, synthetic construct
  source 1..10: mol_type = other DNA
gtaagtgtac

SEQ ID NO: 798 - DNA, 10 bp, synthetic construct
  source 1..10: mol_type = other DNA
tagtcacgtt

SEQ ID NO: 799 - DNA, 10 bp, synthetic construct
  source 1..10: mol_type = other DNA
cctgtcacca

SEQ ID NO: 800 - DNA, 10 bp, synthetic construct
  source 1..10: mol_type = other DNA
atcgtggatg

SEQ ID NO: 801 - DNA, 10 bp, synthetic construct
  source 1..10: mol_type = other DNA
tggagatcga

SEQ ID NO: 802 - DNA, 10 bp, synthetic construct
  source 1..10: mol_type = other DNA
cctcacagat

SEQ ID NO: 803 - DNA, 10 bp, synthetic construct
  source 1..10: mol_type = other DNA
gaatctctcc

SEQ ID NO: 804 - DNA, 10 bp, synthetic construct
  source 1..10: mol_type = other DNA
gcagactgac

SEQ ID NO: 805 - DNA, 10 bp, synthetic construct
  source 1..10: mol_type = other DNA
ctcattaacg

SEQ ID NO: 806 - DNA, 10 bp, synthetic construct
  source 1..10: mol_type = other DNA
tggtgagctt

SEQ ID NO: 807 - DNA, 10 bp, synthetic construct
  source 1..10: mol_type = other DNA
aatccgctga

SEQ ID NO: 808 - DNA, 10 bp, synthetic construct
  source 1..10: mol_type = other DNA
tcgcatcaac

SEQ ID NO: 809 - DNA, 10 bp, synthetic construct
  source 1..10: mol_type = other DNA
agaacagtga

SEQ ID NO: 810 - DNA, 10 bp, synthetic construct
  source 1..10: mol_type = other DNA
catgtctcct

SEQ ID NO: 811 - DNA, 10 bp, synthetic construct
  source 1..10: mol_type = other DNA
gtctggagtg

SEQ ID NO: 812 - DNA, 10 bp, synthetic construct
  source 1..10: mol_type = other DNA
gaggtctgtg

SEQ ID NO: 813 - DNA, 10 bp, synthetic construct
  source 1..10: mol_type = other DNA
ctatagacgt

SEQ ID NO: 814 - DNA, 10 bp, synthetic construct
  source 1..10: mol_type = other DNA
tgcagtgacc

SEQ ID NO: 815 - DNA, 10 bp, synthetic construct
  source 1..10: mol_type = other DNA
actccactaa

SEQ ID NO: 816 - DNA, 10 bp, synthetic construct
  source 1..10: mol_type = other DNA
gcgaagtagg

SEQ ID NO: 817 - DNA, 10 bp, synthetic construct
  source 1..10: mol_type = other DNA
tgcctaacct

SEQ ID NO: 818 - DNA, 10 bp, synthetic construct
  source 1..10: mol_type = other DNA
aatggtctac

SEQ ID NO: 819 - DNA, 10 bp, synthetic construct
  source 1..10: mol_type = other DNA
ctatccggta

SEQ ID NO: 820 - DNA, 10 bp, synthetic construct
  source 1..10: mol_type = other DNA
tacgcttcag

SEQ ID NO: 821 - DNA, 10 bp, synthetic construct
  source 1..10: mol_type = other DNA
cggagcatct

SEQ ID NO: 822 - DNA, 10 bp, synthetic construct
  source 1..10: mol_type = other DNA
gtactagatc

SEQ ID NO: 823 - DNA, 10 bp, synthetic construct
  source 1..10: mol_type = other DNA
acttagcgga

SEQ ID NO: 824 - DNA, 10 bp, synthetic construct
  source 1..10: mol_type = other DNA
atcactccat

SEQ ID NO: 825 - DNA, 10 bp, synthetic construct
  source 1..10: mol_type = other DNA
gatcgcagtg

SEQ ID NO: 826 - DNA, 10 bp, synthetic construct
  source 1..10: mol_type = other DNA
ccggaattcc

SEQ ID NO: 827 - DNA, 10 bp, synthetic construct
  source 1..10: mol_type = other DNA
tgattggaga

SEQ ID NO: 828 - DNA, 10 bp, synthetic construct
  source 1..10: mol_type = other DNA
cacaaggtcg

SEQ ID NO: 829 - DNA, 10 bp, synthetic construct
  source 1..10: mol_type = other DNA
tctcgcagga

SEQ ID NO: 830 - DNA, 10 bp, synthetic construct
  source 1..10: mol_type = other DNA
gtggtatcat

SEQ ID NO: 831 - DNA, 10 bp, synthetic construct
  source 1..10: mol_type = other DNA
agatctcatc

SEQ ID NO: 832 - DNA, 10 bp, synthetic construct
  source 1..10: mol_type = other DNA
gatggagatt

SEQ ID NO: 833 - DNA, 10 bp, synthetic construct
  source 1..10: mol_type = other DNA
ctcattctgc

SEQ ID NO: 834 - DNA, 10 bp, synthetic construct
  source 1..10: mol_type = other DNA
tggcagacaa

SEQ ID NO: 835 - DNA, 10 bp, synthetic construct
  source 1..10: mol_type = other DNA
acatcctgcg

SEQ ID NO: 836 - DNA, 10 bp, synthetic construct
  source 1..10: mol_type = other DNA
acgtcgcaga

SEQ ID NO: 837 - DNA, 10 bp, synthetic construct
  source 1..10: mol_type = other DNA
tgtataggct

SEQ ID NO: 838 - DNA, 10 bp, synthetic construct
  source 1..10: mol_type = other DNA
caacacattg

SEQ ID NO: 839 - DNA, 10 bp, synthetic construct
  source 1..10: mol_type = other DNA
gtcggttcac

SEQ ID NO: 840 - DNA, 10 bp, synthetic construct
  source 1..10: mol_type = other DNA
agccataagc

SEQ ID NO: 841 - DNA, 10 bp, synthetic construct
  source 1..10: mol_type = other DNA
gtattccgag

SEQ ID NO: 842 - DNA, 10 bp, synthetic construct
  source 1..10: mol_type = other DNA
cataggttca

SEQ ID NO: 843 - DNA, 10 bp, synthetic construct
  source 1..10: mol_type = other DNA
tcggcagctt

SEQ ID NO: 844 - DNA, 10 bp, synthetic construct
  source 1..10: mol_type = other DNA
gttcggtcct

SEQ ID NO: 845 - DNA, 10 bp, synthetic construct
  source 1..10: mol_type = other DNA
tggattgtag

SEQ ID NO: 846 - DNA, 10 bp, synthetic construct
  source 1..10: mol_type = other DNA
ccagaacgtc

SEQ ID NO: 847 - DNA, 10 bp, synthetic construct
  source 1..10: mol_type = other DNA
aactccaaga

SEQ ID NO: 848 - DNA, 10 bp, synthetic construct
  source 1..10: mol_type = other DNA
cgtacactgg

SEQ ID NO: 849 - DNA, 10 bp, synthetic construct
  source 1..10: mol_type = other DNA
gcacacagca

SEQ ID NO: 850 - DNA, 10 bp, synthetic construct
  source 1..10: mol_type = other DNA
atggtgtatc

SEQ ID NO: 851 - DNA, 10 bp, synthetic construct
  source 1..10: mol_type = other DNA
tactgtgcat

SEQ ID NO: 852 - DNA, 10 bp, synthetic construct
  source 1..10: mol_type = other DNA
cggcaatcag

SEQ ID NO: 853 - DNA, 10 bp, synthetic construct
  source 1..10: mol_type = other DNA
gtagttcgga

SEQ ID NO: 854 - DNA, 10 bp, synthetic construct
  source 1..10: mol_type = other DNA
tacaggaact

SEQ ID NO: 855 - DNA, 10 bp, synthetic construct
  source 1..10: mol_type = other DNA
acttccgttc

SEQ ID NO: 856 - DNA, 10 bp, synthetic construct
  source 1..10: mol_type = other DNA
tgctccacga

SEQ ID NO: 857 - DNA, 10 bp, synthetic construct
  source 1..10: mol_type = other DNA
aatcaaggtc

SEQ ID NO: 858 - DNA, 10 bp, synthetic construct
  source 1..10: mol_type = other DNA
gtaggtcaat

SEQ ID NO: 859 - DNA, 10 bp, synthetic construct
  source 1..10: mol_type = other DNA
ccgatgttcg

SEQ ID NO: 860 - DNA, 10 bp, synthetic construct
  source 1..10: mol_type = other DNA
gacgtgtgca

SEQ ID NO: 861 - DNA, 10 bp, synthetic construct
  source 1..10: mol_type = other DNA
tcgccacttc

SEQ ID NO: 862 - DNA, 10 bp, synthetic construct
  source 1..10: mol_type = other DNA
ataagcacgt

SEQ ID NO: 863 - DNA, 10 bp, synthetic construct
  source 1..10: mol_type = other DNA
cgttatgaag

SEQ ID NO: 864 - DNA, 10 bp, synthetic construct
  source 1..10: mol_type = other DNA
aatagagcca

SEQ ID NO: 865 - DNA, 10 bp, synthetic construct
  source 1..10: mol_type = other DNA
gtacctcgac

SEQ ID NO: 866 - DNA, 10 bp, synthetic construct
  source 1..10: mol_type = other DNA
ccggtgattg

SEQ ID NO: 867 - DNA, 10 bp, synthetic construct
  source 1..10: mol_type = other DNA
tgctactagt

SEQ ID NO: 868 - DNA, 10 bp, synthetic construct
  source 1..10: mol_type = other DNA
gatccggact

SEQ ID NO: 869 - DNA, 10 bp, synthetic construct
  source 1..10: mol_type = other DNA
ttaggcacaa

SEQ ID NO: 870 - DNA, 10 bp, synthetic construct
  source 1..10: mol_type = other DNA
agcattcttg

SEQ ID NO: 871 - DNA, 10 bp, synthetic construct
  source 1..10: mol_type = other DNA
ccgtaatggc

SEQ ID NO: 872 - DNA, 10 bp, synthetic construct
  source 1..10: mol_type = other DNA
gtatagctgc

SEQ ID NO: 873 - DNA, 10 bp, synthetic construct
  source 1..10: mol_type = other DNA
cagacatctg

SEQ ID NO: 874 - DNA, 10 bp, synthetic construct
  source 1..10: mol_type = other DNA
agcggtggat

SEQ ID NO: 875 - DNA, 10 bp, synthetic construct
  source 1..10: mol_type = other DNA
tctctcaaca

SEQ ID NO: 876 - DNA, 10 bp, synthetic construct
  source 1..10: mol_type = other DNA
catccactgt

SEQ ID NO: 877 - DNA, 10 bp, synthetic construct
  source 1..10: mol_type = other DNA
gccgtgaaca

SEQ ID NO: 878 - DNA, 10 bp, synthetic construct
  source 1..10: mol_type = other DNA
ataagcggag

SEQ ID NO: 879 - DNA, 10 bp, synthetic construct
  source 1..10: mol_type = other DNA
tggtattctc

SEQ ID NO: 880 - DNA, 10 bp, synthetic construct
  source 1..10: mol_type = other DNA
acagttctca

SEQ ID NO: 881 - DNA, 10 bp, synthetic construct
  source 1..10: mol_type = other DNA
cattgagagc

SEQ ID NO: 882 - DNA, 10 bp, synthetic construct
  source 1..10: mol_type = other DNA
gtcacgactg

SEQ ID NO: 883 - DNA, 10 bp, synthetic construct
  source 1..10: mol_type = other DNA
tggcactgat

SEQ ID NO: 884 - DNA, 10 bp, synthetic construct
  source 1..10: mol_type = other DNA
aatgattcgc

SEQ ID NO: 885 - DNA, 10 bp, synthetic construct
  source 1..10: mol_type = other DNA
cgcttaagta

SEQ ID NO: 886 - DNA, 10 bp, synthetic construct
  source 1..10: mol_type = other DNA
ttgagcgacg

SEQ ID NO: 887 - DNA, 10 bp, synthetic construct
  source 1..10: mol_type = other DNA
gcaccgctat

SEQ ID NO: 888 - DNA, 10 bp, synthetic construct
  source 1..10: mol_type = other DNA
acgaacggat

SEQ ID NO: 889 - DNA, 10 bp, synthetic construct
  source 1..10: mol_type = other DNA
gaacggacta

SEQ ID NO: 890 - DNA, 10 bp, synthetic construct
  source 1..10: mol_type = other DNA
tgctctctgg

SEQ ID NO: 891 - DNA, 10 bp, synthetic construct
  source 1..10: mol_type = other DNA
cttgtatacc

SEQ ID NO: 892 - DNA, 10 bp, synthetic construct
  source 1..10: mol_type = other DNA
caccagcaca

SEQ ID NO: 893 - DNA, 10 bp, synthetic construct
  source 1..10: mol_type = other DNA
ttgtctgtag

SEQ ID NO: 894 - DNA, 10 bp, synthetic construct
  source 1..10: mol_type = other DNA
agtatcactc

SEQ ID NO: 895 - DNA, 10 bp, synthetic construct
  source 1..10: mol_type = other DNA
gcaggatggt

SEQ ID NO: 896 - DNA, 10 bp, synthetic construct
  source 1..10: mol_type = other DNA
ttatccacgt

SEQ ID NO: 897 - DNA, 10 bp, synthetic construct
  source 1..10: mol_type = other DNA
acggttcgtc

SEQ ID NO: 898 - DNA, 10 bp, synthetic construct
  source 1..10: mol_type = other DNA
gaccagtaag

SEQ ID NO: 899 - DNA, 10 bp, synthetic construct
  source 1..10: mol_type = other DNA
cgtagagtca

SEQ ID NO: 900 - DNA, 10 bp, synthetic construct
  source 1..10: mol_type = other DNA
acgttaaggt

SEQ ID NO: 901 - DNA, 10 bp, synthetic construct
  source 1..10: mol_type = other DNA
ctcagcttag

SEQ ID NO: 902 - DNA, 10 bp, synthetic construct
  source 1..10: mol_type = other DNA
tgtgctccta

SEQ ID NO: 903 - DNA, 10 bp, synthetic construct
  source 1..10: mol_type = other DNA
gaacaggacc

SEQ ID NO: 904 - DNA, 10 bp, synthetic construct
  source 1..10: mol_type = other DNA
gcgaccttga

SEQ ID NO: 905 - DNA, 10 bp, synthetic construct
  source 1..10: mol_type = other DNA
atcgtgagtt

SEQ ID NO: 906 - DNA, 10 bp, synthetic construct
  source 1..10: mol_type = other DNA
cgtcgtcaac

SEQ ID NO: 907 - DNA, 10 bp, synthetic construct
  source 1..10: mol_type = other DNA
taataagccg

SEQ ID NO: 908 - DNA, 10 bp, synthetic construct
  source 1..10: mol_type = other DNA
ccacttagta

SEQ ID NO: 909 - DNA, 10 bp, synthetic construct
  source 1..10: mol_type = other DNA
agtagctagt

SEQ ID NO: 910 - DNA, 10 bp, synthetic construct
  source 1..10: mol_type = other DNA
gacgaactcg

SEQ ID NO: 911 - DNA, 10 bp, synthetic construct
  source 1..10: mol_type = other DNA
ttgtcggcac

SEQ ID NO: 912 - DNA, 10 bp, synthetic construct
  source 1..10: mol_type = other DNA
agcatatcgt

SEQ ID NO: 913 - DNA, 10 bp, synthetic construct
  source 1..10: mol_type = other DNA
gtagacggag

SEQ ID NO: 914 - DNA, 10 bp, synthetic construct
  source 1..10: mol_type = other DNA
cattctcatc

SEQ ID NO: 915 - DNA, 10 bp, synthetic construct
  source 1..10: mol_type = other DNA
tcgcggatca

SEQ ID NO: 916 - DNA, 10 bp, synthetic construct
  source 1..10: mol_type = other DNA
ctggaggcaa

SEQ ID NO: 917 - DNA, 10 bp, synthetic construct
  source 1..10: mol_type = other DNA
gatacttgtg

SEQ ID NO: 918 - DNA, 10 bp, synthetic construct
  source 1..10: mol_type = other DNA
tgcctccact

SEQ ID NO: 919 - DNA, 10 bp, synthetic construct
  source 1..10: mol_type = other DNA
acatgaatgc

SEQ ID NO: 920 - DNA, 10 bp, synthetic construct
  source 1..10: mol_type = other DNA
tcagcagagg

SEQ ID NO: 921 - DNA, 10 bp, synthetic construct
  source 1..10: mol_type = other DNA
ctgtgcatta

SEQ ID NO: 922 - DNA, 10 bp, synthetic construct
  source 1..10: mol_type = other DNA
agtaatcgac

SEQ ID NO: 923 - DNA, 10 bp, synthetic construct
  source 1..10: mol_type = other DNA
agtaatcgac

SEQ ID NO: 924 - DNA, 10 bp, synthetic construct
  source 1..10: mol_type = other DNA
aagcggtgaa

SEQ ID NO: 925 - DNA, 10 bp, synthetic construct
  source 1..10: mol_type = other DNA
ctatcacact

SEQ ID NO: 926 - DNA, 10 bp, synthetic construct
  source 1..10: mol_type = other DNA
gccgttatgc

SEQ ID NO: 927 - DNA, 10 bp, synthetic construct
  source 1..10: mol_type = other DNA
tgtaacgctg

SEQ ID NO: 928 - DNA, 10 bp, synthetic construct
  source 1..10: mol_type = other DNA
gcgtgtaact

SEQ ID NO: 929 - DNA, 10 bp, synthetic construct
  source 1..10: mol_type = other DNA
catgtaccac

SEQ ID NO: 930 - DNA, 10 bp, synthetic construct
  source 1..10: mol_type = other DNA
tgccactgta

SEQ ID NO: 931 - DNA, 10 bp, synthetic construct
  source 1..10: mol_type = other DNA
ataacggtgg

SEQ ID NO: 932 - DNA, 10 bp, synthetic construct
  source 1..10: mol_type = other DNA
cttgaaggtt

SEQ ID NO: 933 - DNA, 10 bp, synthetic construct
  source 1..10: mol_type = other DNA
gcctctatgg

SEQ ID NO: 934 - DNA, 10 bp, synthetic construct
  source 1..10: mol_type = other DNA
tagatccacc

SEQ ID NO: 935 - DNA, 10 bp, synthetic construct
  source 1..10: mol_type = other DNA
agacggtcaa

SEQ ID NO: 936 - DNA, 10 bp, synthetic construct
  source 1..10: mol_type = other DNA
gctggattaa

SEQ ID NO: 937 - DNA, 10 bp, synthetic construct
  source 1..10: mol_type = other DNA
taacaccggc

SEQ ID NO: 938 - DNA, 10 bp, synthetic construct
  source 1..10: mol_type = other DNA
atgactgccg

SEQ ID NO: 939 - DNA, 10 bp, synthetic construct
  source 1..10: mol_type = other DNA
cgcttgaatt

SEQ ID NO: 940 - DNA, 10 bp, synthetic construct
  source 1..10: mol_type = other DNA
ctatagcgag

SEQ ID NO: 941 - DNA, 10 bp, synthetic construct
  source 1..10: mol_type = other DNA
aacgttgttc

SEQ ID NO: 942 - DNA, 10 bp, synthetic construct
  source 1..10: mol_type = other DNA
gctaccacgt

SEQ ID NO: 943 - DNA, 10 bp, synthetic construct
  source 1..10: mol_type = other DNA
tggcgataca

SEQ ID NO: 944 - DNA, 10 bp, synthetic construct
  source 1..10: mol_type = other DNA
tccgccaatc

SEQ ID NO: 945 - DNA, 10 bp, synthetic construct
  source 1..10: mol_type = other DNA
caactagtgt

SEQ ID NO: 946 - DNA, 10 bp, synthetic construct
  source 1..10: mol_type = other DNA
agtaagccaa

SEQ ID NO: 947 - DNA, 10 bp, synthetic construct
  source 1..10: mol_type = other DNA
gtgtgttgcg

SEQ ID NO: 948 - DNA, 10 bp, synthetic construct
  source 1..10: mol_type = other DNA
gatcagatgg

SEQ ID NO: 949 - DNA, 10 bp, synthetic construct
  source 1..10: mol_type = other DNA
ctcgtaggtc

SEQ ID NO: 950 - DNA, 10 bp, synthetic construct
  source 1..10: mol_type = other DNA
tcgtgtccat

SEQ ID NO: 951 - DNA, 10 bp, synthetic construct
  source 1..10: mol_type = other DNA
agaacctaca

SEQ ID NO: 952 - DNA, 10 bp, synthetic construct
  source 1..10: mol_type = other DNA
ccgcattcct

SEQ ID NO: 953 - DNA, 10 bp, synthetic construct
  source 1..10: mol_type = other DNA
gttggacata

SEQ ID NO: 954 - DNA, 10 bp, synthetic construct
  source 1..10: mol_type = other DNA
tgatcgaggc

SEQ ID NO: 955 - DNA, 10 bp, synthetic construct
  source 1..10: mol_type = other DNA
aacatcgtag

SEQ ID NO: 956 - DNA, 10 bp, synthetic construct
  source 1..10: mol_type = other DNA
gttgcgcgaa

SEQ ID NO: 957 - DNA, 10 bp, synthetic construct
  source 1..10: mol_type = other DNA
acaagtaagc

SEQ ID NO: 958 - DNA, 10 bp, synthetic construct
  source 1..10: mol_type = other DNA
cgcttctcct

SEQ ID NO: 959 - DNA, 10 bp, synthetic construct
  source 1..10: mol_type = other DNA
tagcaagttg

SEQ ID NO: 960 - DNA, 10 bp, synthetic construct
  source 1..10: mol_type = other DNA
aggcctcttc

SEQ ID NO: 961 - DNA, 10 bp, synthetic construct
  source 1..10: mol_type = other DNA
gtaatgtcgt

SEQ ID NO: 962 - DNA, 10 bp, synthetic construct
  source 1..10: mol_type = other DNA
cactgcagag

SEQ ID NO: 963 - DNA, 10 bp, synthetic construct
  source 1..10: mol_type = other DNA
tctgaagaca

SEQ ID NO: 964 - DNA, 10 bp, synthetic construct
  source 1..10: mol_type = other DNA
gctaaggata

SEQ ID NO: 965 - DNA, 10 bp, synthetic construct
  source 1..10: mol_type = other DNA
cacggttggt

SEQ ID NO: 966 - DNA, 10 bp, synthetic construct
  source 1..10: mol_type = other DNA
tgaccaccag

SEQ ID NO: 967 - DNA, 10 bp, synthetic construct
  source 1..10: mol_type = other DNA
atgttcatcc

SEQ ID NO: 968 - DNA, 10 bp, synthetic construct
  source 1..10: mol_type = other DNA
agctactctg

SEQ ID NO: 969 - DNA, 10 bp, synthetic construct
  source 1..10: mol_type = other DNA
caacgtgagt

SEQ ID NO: 970 - DNA, 10 bp, synthetic construct
  source 1..10: mol_type = other DNA
tcgatgctaa

SEQ ID NO: 971 - DNA, 10 bp, synthetic construct
  source 1..10: mol_type = other DNA
gttgcaagcc

SEQ ID NO: 972 - DNA, 10 bp, synthetic construct
  source 1..10: mol_type = other DNA
cgacatgtgt

SEQ ID NO: 973 - DNA, 10 bp, synthetic construct
  source 1..10: mol_type = other DNA
gatgcgcata

SEQ ID NO: 974 - DNA, 10 bp, synthetic construct
  source 1..10: mol_type = other DNA
tcgtgatcag

SEQ ID NO: 975 - DNA, 10 bp, synthetic construct
  source 1..10: mol_type = other DNA
atcatcagcc

SEQ ID NO: 976 - DNA, 10 bp, synthetic construct
  source 1..10: mol_type = other DNA
tcaatggcgg

SEQ ID NO: 977 - DNA, 10 bp, synthetic construct
  source 1..10: mol_type = other DNA
cagtaactct

SEQ ID NO: 978 - DNA, 10 bp, synthetic construct
  source 1..10: mol_type = other DNA
gttgcctgac

SEQ ID NO: 979 - DNA, 10 bp, synthetic construct
  source 1..10: mol_type = other DNA
agccgtaata

SEQ ID NO: 980 - DNA, 10 bp, synthetic construct
  source 1..10: mol_type = other DNA
ctaataggct

SEQ ID NO: 981 - DNA, 10 bp, synthetic construct
  source 1..10: mol_type = other DNA
tctcctccac

SEQ ID NO: 982 - DNA, 10 bp, synthetic construct
  source 1..10: mol_type = other DNA
agctgcatga

SEQ ID NO: 983 - DNA, 10 bp, synthetic construct
  source 1..10: mol_type = other DNA
gaggagtatg

SEQ ID NO: 984 - DNA, 10 bp, synthetic construct
  source 1..10: mol_type = other DNA
accacgtagc

SEQ ID NO: 985 - DNA, 10 bp, synthetic construct
  source 1..10: mol_type = other DNA
gaatgcagta

SEQ ID NO: 986 - DNA, 10 bp, synthetic construct
  source 1..10: mol_type = other DNA
ttggtagcct

SEQ ID NO: 987 - DNA, 10 bp, synthetic construct
  source 1..10: mol_type = other DNA
cgtcatctag

SEQ ID NO: 988 - DNA, 10 bp, synthetic construct
  source 1..10: mol_type = other DNA
tcaatgaggt

SEQ ID NO: 989 - DNA, 10 bp, synthetic construct
  source 1..10: mol_type = other DNA
agcgaagctg

SEQ ID NO: 990 - DNA, 10 bp, synthetic construct
  source 1..10: mol_type = other DNA
ctgtcttaac

SEQ ID NO: 991 - DNA, 10 bp, synthetic construct
  source 1..10: mol_type = other DNA
gatcgcctca

SEQ ID NO: 992 - DNA, 10 bp, synthetic construct
  source 1..10: mol_type = other DNA
gttccgaatg

SEQ ID NO: 993 - DNA, 10 bp, synthetic construct
  source 1..10: mol_type = other DNA
tacgtacgca

SEQ ID NO: 994 - DNA, 10 bp, synthetic construct
  source 1..10: mol_type = other DNA
cggagttcac

SEQ ID NO: 995 - DNA, 10 bp, synthetic construct
  source 1..10: mol_type = other DNA
acatacgtgt

SEQ ID NO: 996 - DNA, 10 bp, synthetic construct
  source 1..10: mol_type = other DNA
ctgaagagat

SEQ ID NO: 997 - DNA, 10 bp, synthetic construct
  source 1..10: mol_type = other DNA
gaagcctcca

SEQ ID NO: 998 - DNA, 10 bp, synthetic construct
  source 1..10: mol_type = other DNA
tgtcttcatc

SEQ ID NO: 999 - DNA, 10 bp, synthetic construct
  source 1..10: mol_type = other DNA
acctgagtgg

SEQ ID NO: 1000 - DNA, 10 bp, synthetic construct
  source 1..10: mol_type = other DNA
gtacgtcctt

SEQ ID NO: 1001 - DNA, 10 bp, synthetic construct
  source 1..10: mol_type = other DNA
aactaggtca

SEQ ID NO: 1002 - DNA, 10 bp, synthetic construct
  source 1..10: mol_type = other DNA
tgtgtcaggc

SEQ ID NO: 1003 - DNA, 10 bp, synthetic construct
  source 1..10: mol_type = other DNA
ccgacataag

SEQ ID NO: 1004 - DNA, 10 bp, synthetic construct
  source 1..10: mol_type = other DNA
tccacacgtc

SEQ ID NO: 1005 - DNA, 10 bp, synthetic construct
  source 1..10: mol_type = other DNA
cggcacatga

SEQ ID NO: 1006 - DNA, 10 bp, synthetic construct
  source 1..10: mol_type = other DNA
gtatgttcct

SEQ ID NO: 1007 - DNA, 10 bp, synthetic construct
  source 1..10: mol_type = other DNA
aatgtggaag

SEQ ID NO: 1008 - DNA, 10 bp, synthetic construct
  source 1..10: mol_type = other DNA
agacagacgt

SEQ ID NO: 1009 - DNA, 10 bp, synthetic construct
  source 1..10: mol_type = other DNA
ttctgtggag

SEQ ID NO: 1010 - DNA, 10 bp, synthetic construct
  source 1..10: mol_type = other DNA
caggtcttcc

SEQ ID NO: 1011 - DNA, 10 bp, synthetic construct
  source 1..10: mol_type = other DNA
gctacacata

SEQ ID NO: 1012 - DNA, 10 bp, synthetic construct
  source 1..10: mol_type = other DNA
ctagcgacac

SEQ ID NO: 1013 - DNA, 10 bp, synthetic construct
  source 1..10: mol_type = other DNA
aggcattact

SEQ ID NO: 1014 - DNA, 10 bp, synthetic construct
  source 1..10: mol_type = other DNA
gacatccgga

SEQ ID NO: 1015 - DNA, 10 bp, synthetic construct
  source 1..10: mol_type = other DNA
tcttgagttg

SEQ ID NO: 1016 - DNA, 10 bp, synthetic construct
  source 1..10: mol_type = other DNA
acaacagaag

SEQ ID NO: 1017 - DNA, 10 bp, synthetic construct
  source 1..10: mol_type = other DNA
cgcttgtgga

SEQ ID NO: 1018 - DNA, 10 bp, synthetic construct
  source 1..10: mol_type = other DNA
gttggtctcc

SEQ ID NO: 1019 - DNA, 10 bp, synthetic construct
  source 1..10: mol_type = other DNA
tagcacactt

SEQ ID NO: 1020 - DNA, 10 bp, synthetic construct
  source 1..10: mol_type = other DNA
gcttgcaata

SEQ ID NO: 1021 - DNA, 10 bp, synthetic construct
  source 1..10: mol_type = other DNA
tggacgtgct

SEQ ID NO: 1022 - DNA, 10 bp, synthetic construct
  source 1..10: mol_type = other DNA
aacgaaccgg

SEQ ID NO: 1023 - DNA, 10 bp, synthetic construct
  source 1..10: mol_type = other DNA
ctacttgtac

SEQ ID NO: 1024 - DNA, 10 bp, synthetic construct
  source 1..10: mol_type = other DNA
cggtgagtga

SEQ ID NO: 1025 - DNA, 10 bp, synthetic construct
  source 1..10: mol_type = other DNA
gcaatgcatt

SEQ ID NO: 1026 - DNA, 10 bp, synthetic construct
  source 1..10: mol_type = other DNA
ttccattcac

SEQ ID NO: 1027 - DNA, 10 bp, synthetic construct
  source 1..10: mol_type = other DNA
aatgccagcg

SEQ ID NO: 1028 - DNA, 10 bp, synthetic construct
  source 1..10: mol_type = other DNA
tactcttctc

SEQ ID NO: 1029 - DNA, 10 bp, synthetic construct
  source 1..10: mol_type = other DNA
aggaaggtaa

SEQ ID NO: 1030 - DNA, 10 bp, synthetic construct
  source 1..10: mol_type = other DNA
gttggacggt

SEQ ID NO: 1031 - DNA, 10 bp, synthetic construct
  source 1..10: mol_type = other DNA
ccactcaacg

SEQ ID NO: 1032 - DNA, 10 bp, synthetic construct
  source 1..10: mol_type = other DNA
aatgactggt

SEQ ID NO: 1033 - DNA, 10 bp, synthetic construct
  source 1..10: mol_type = other DNA
gtgccacaac

SEQ ID NO: 1034 - DNA, 10 bp, synthetic construct
  source 1..10: mol_type = other DNA
cgaatgatcg

SEQ ID NO: 1035 - DNA, 10 bp, synthetic construct
  source 1..10: mol_type = other DNA
tcctgtgcta

SEQ ID NO: 1036 - DNA, 10 bp, synthetic construct
  source 1..10: mol_type = other DNA
tgtgaattgg

SEQ ID NO: 1037 - DNA, 10 bp, synthetic construct
  source 1..10: mol_type = other DNA
aaccggcctt

SEQ ID NO: 1038 - DNA, 10 bp, synthetic construct
  source 1..10: mol_type = other DNA
ctatccgacc

SEQ ID NO: 1039 - DNA, 10 bp, synthetic construct
  source 1..10: mol_type = other DNA
gcgattagaa

SEQ ID NO: 1040 - DNA, 10 bp, synthetic construct
  source 1..10: mol_type = other DNA
cgtaaccgca

SEQ ID NO: 1041 - DNA, 10 bp, synthetic construct
  source 1..10: mol_type = other DNA
gactgataac

SEQ ID NO: 1042 - DNA, 10 bp, synthetic construct
  source 1..10: mol_type = other DNA
atgcttactg

SEQ ID NO: 1043 - DNA, 10 bp, synthetic construct
  source 1..10: mol_type = other DNA
tcagcggtgt

SEQ ID NO: 1044 - DNA, 10 bp, synthetic construct
  source 1..10: mol_type = other DNA
acataacacc

SEQ ID NO: 1045 - DNA, 10 bp, synthetic construct
  source 1..10: mol_type = other DNA
cacctgaggt

SEQ ID NO: 1046 - DNA, 10 bp, synthetic construct
  source 1..10: mol_type = other DNA
gtgactgtaa

SEQ ID NO: 1047 - DNA, 10 bp, synthetic construct
  source 1..10: mol_type = other DNA
tgtggctctg

SEQ ID NO: 1048 - DNA, 10 bp, synthetic construct
  source 1..10: mol_type = other DNA
ctcagactct

SEQ ID NO: 1049 - DNA, 10 bp, synthetic construct
  source 1..10: mol_type = other DNA
acacatgcta

SEQ ID NO: 1050 - DNA, 10 bp, synthetic construct
  source 1..10: mol_type = other DNA
tgtgcctaag

SEQ ID NO: 1051 - DNA, 10 bp, synthetic construct
  source 1..10: mol_type = other DNA
gagttgaggc

SEQ ID NO: 1052 - DNA, 10 bp, synthetic construct
  source 1..10: mol_type = other DNA
agccgttctc

SEQ ID NO: 1053 - DNA, 10 bp, synthetic construct
  source 1..10: mol_type = other DNA
ttgttggtct

SEQ ID NO: 1054 - DNA, 10 bp, synthetic construct
  source 1..10: mol_type = other DNA
caagacaaga

SEQ ID NO: 1055 - DNA, 10 bp, synthetic construct
  source 1..10: mol_type = other DNA
gctacacgag

SEQ ID NO: 1056 - DNA, 10 bp, synthetic construct
  source 1..10: mol_type = other DNA
gtgacgcgat

SEQ ID NO: 1057 - DNA, 10 bp, synthetic construct
  source 1..10: mol_type = other DNA
aacctctctg

SEQ ID NO: 1058 - DNA, 10 bp, synthetic construct
  source 1..10: mol_type = other DNA
tcttgagaga

SEQ ID NO: 1059 - DNA, 10 bp, synthetic construct
  source 1..10: mol_type = other DNA
cgagatatcc

SEQ ID NO: 1060 - DNA, 10 bp, synthetic construct
  source 1..10: mol_type = other DNA
gaaggattca

SEQ ID NO: 1061 - DNA, 10 bp, synthetic construct
  source 1..10: mol_type = other DNA
tcgcctggtt

SEQ ID NO: 1062 - DNA, 10 bp, synthetic construct
  source 1..10: mol_type = other DNA
agtaacacgg

SEQ ID NO: 1063 - DNA, 10 bp, synthetic construct
  source 1..10: mol_type = other DNA
ctcttgcaac

SEQ ID NO: 1064 - DNA, 10 bp, synthetic construct
  source 1..10: mol_type = other DNA
agagacttac

SEQ ID NO: 1065 - DNA, 10 bp, synthetic construct
  source 1..10: mol_type = other DNA
cttatggccg

SEQ ID NO: 1066 - DNA, 10 bp, synthetic construct
  source 1..10: mol_type = other DNA
gcctgacgtt

SEQ ID NO: 1067 - DNA, 10 bp, synthetic construct
  source 1..10: mol_type = other DNA
tagcctaaga

SEQ ID NO: 1068 - DNA, 10 bp, synthetic construct
  source 1..10: mol_type = other DNA
aagcatatcc

SEQ ID NO: 1069 - DNA, 10 bp, synthetic construct
  source 1..10: mol_type = other DNA
ctaatccgtt

SEQ ID NO: 1070 - DNA, 10 bp, synthetic construct
  source 1..10: mol_type = other DNA
gcttggtcga

SEQ ID NO: 1071 - DNA, 10 bp, synthetic construct
  source 1..10: mol_type = other DNA
tgcgcagaag

SEQ ID NO: 1072 - DNA, 10 bp, synthetic construct
  source 1..10: mol_type = other DNA
gatcctgata

SEQ ID NO: 1073 - DNA, 10 bp, synthetic construct
  source 1..10: mol_type = other DNA
tcaagcacgg

SEQ ID NO: 1074 - DNA, 10 bp, synthetic construct
  source 1..10: mol_type = other DNA
ctgtagcgct

SEQ ID NO: 1075 - DNA, 10 bp, synthetic construct
  source 1..10: mol_type = other DNA
agcgtattac

SEQ ID NO: 1076 - DNA, 10 bp, synthetic construct
  source 1..10: mol_type = other DNA
tgtctgattg

SEQ ID NO: 1077 - DNA, 10 bp, synthetic construct
  source 1..10: mol_type = other DNA
accagacggt

SEQ ID NO: 1078 - DNA, 10 bp, synthetic construct
  misc_feature 1..10
  source 1..10: mol_type = other DNA
gtgtctgacc

SEQ ID NO: 1079 - DNA, 10 bp, synthetic construct
  source 1..10: mol_type = other DNA
caagactcaa

SEQ ID NO: 1080 - DNA, 10 bp, synthetic construct
  source 1..10: mol_type = other DNA
caagactcaa

SEQ ID NO: 1081 - DNA, 10 bp, synthetic construct
  source 1..10: mol_type = other DNA
agacgaggtc

SEQ ID NO: 1082 - DNA, 10 bp, synthetic construct
  source 1..10: mol_type = other DNA
ctggattact

SEQ ID NO: 1083 - DNA, 10 bp, synthetic construct
  source 1..10: mol_type = other DNA
gatatgctga

SEQ ID NO: 1084 - DNA, 10 bp, synthetic construct
  source 1..10: mol_type = other DNA
ctggtcaagg

SEQ ID NO: 1085 - DNA, 10 bp, synthetic construct
  source 1..10: mol_type = other DNA
gctccattcc

SEQ ID NO: 1086 - DNA, 10 bp, synthetic construct
  source 1..10: mol_type = other DNA
tgatgtcgaa

SEQ ID NO: 1087 - DNA, 10 bp, synthetic construct
  source 1..10: mol_type = other DNA
aacaaggctt

SEQ ID NO: 1088 - DNA, 10 bp, synthetic construct
  source 1..10: mol_type = other DNA
catctagaca

SEQ ID NO: 1089 - DNA, 10 bp, synthetic construct
  source 1..10: mol_type = other DNA
gcggatacag

SEQ ID NO: 1090 - DNA, 10 bp, synthetic construct
  source 1..10: mol_type = other DNA
ttctgcttgt

SEQ ID NO: 1091 - DNA, 10 bp, synthetic construct
  source 1..10: mol_type = other DNA
agaacgcgtc

SEQ ID NO: 1092 - DNA, 10 bp, synthetic construct
  source 1..10: mol_type = other DNA
aagacgaact

SEQ ID NO: 1093 - DNA, 10 bp, synthetic construct
  source 1..10: mol_type = other DNA
ccagtactgc

SEQ ID NO: 1094 - DNA, 10 bp, synthetic construct
  source 1..10: mol_type = other DNA
gttcgctgta

SEQ ID NO: 1095 - DNA, 10 bp, synthetic construct
  source 1..10: mol_type = other DNA
tgctatgcag

SEQ ID NO: 1096 - DNA, 10 bp, synthetic construct
  source 1..10: mol_type = other DNA
tacacgcgca

SEQ ID NO: 1097 - DNA, 10 bp, synthetic construct
  source 1..10: mol_type = other DNA
aggtacgcag

SEQ ID NO: 1098 - DNA, 10 bp, synthetic construct
  source 1..10: mol_type = other DNA
gttcgattgt

SEQ ID NO: 1099 - DNA, 10 bp, synthetic construct
  source 1..10: mol_type = other DNA
ccagttaatc

SEQ ID NO: 1100 - DNA, 10 bp, synthetic construct
  source 1..10: mol_type = other DNA
taagatcgga

SEQ ID NO: 1101 - DNA, 10 bp, synthetic construct
  source 1..10: mol_type = other DNA
agctcggctt

SEQ ID NO: 1102 - DNA, 10 bp, synthetic construct
  source 1..10: mol_type = other DNA
gttcgataag

SEQ ID NO: 1103 - DNA, 10 bp, synthetic construct
  source 1..10: mol_type = other DNA
ccgatcatcc

SEQ ID NO: 1104 - DNA, 10 bp, synthetic construct
  source 1..10: mol_type = other DNA
actggactca

SEQ ID NO: 1105 - DNA, 10 bp, synthetic construct
  source 1..10: mol_type = other DNA
caatagaggc

SEQ ID NO: 1106 - DNA, 10 bp, synthetic construct
  source 1..10: mol_type = other DNA
gtcacttaag

SEQ ID NO: 1107 - DNA, 10 bp, synthetic construct
  source 1..10: mol_type = other DNA
tggctcgctt

SEQ ID NO: 1108 - DNA, 10 bp, synthetic construct
  source 1..10: mol_type = other DNA
cgcactatgg

SEQ ID NO: 1109 - DNA, 10 bp, synthetic construct
  source 1..10: mol_type = other DNA
gaggtacatt

SEQ ID NO: 1110 - DNA, 10 bp, synthetic construct
  source 1..10: mol_type = other DNA
tcttagtgac

SEQ ID NO: 1111 - DNA, 10 bp, synthetic construct
  source 1..10: mol_type = other DNA
atacgcgcca

SEQ ID NO: 1112 - DNA, 10 bp, synthetic construct
  source 1..10: mol_type = other DNA
tgaggcatat

SEQ ID NO: 1113 - DNA, 10 bp, synthetic construct
  source 1..10: mol_type = other DNA
attctatggc

SEQ ID NO: 1114 - DNA, 10 bp, synthetic construct
  source 1..10: mol_type = other DNA
ccgtagcatg

SEQ ID NO: 1115 - DNA, 10 bp, synthetic construct
  source 1..10: mol_type = other DNA
gacactgcca

SEQ ID NO: 1116 - DNA, 10 bp, synthetic construct
  source 1..10: mol_type = other DNA
acggcattaa

SEQ ID NO: 1117 - DNA, 10 bp, synthetic construct
  source 1..10: mol_type = other DNA
gtaagcgagg

SEQ ID NO: 1118 - DNA, 10 bp, synthetic construct
  source 1..10: mol_type = other DNA
cattatcgct

SEQ ID NO: 1119 - DNA, 10 bp, synthetic construct
  source 1..10: mol_type = other DNA
tgcctgactc

SEQ ID NO: 1120 - DNA, 10 bp, synthetic construct
  source 1..10: mol_type = other DNA
gactcatcca

SEQ ID NO: 1121 - DNA, 10 bp, synthetic construct
  source 1..10: mol_type = other DNA
acgaacatac

SEQ ID NO: 1122 - DNA, 10 bp, synthetic construct
  source 1..10: mol_type = other DNA
ttacgtcggt

SEQ ID NO: 1123 - DNA, 10 bp, synthetic construct
  source 1..10: mol_type = other DNA
cgtgtggatg

SEQ ID NO: 1124 - DNA, 10 bp, synthetic construct
  source 1..10: mol_type = other DNA
cctaacattc

SEQ ID NO: 1125 - DNA, 10 bp, synthetic construct
  source 1..10: mol_type = other DNA
atcgcacgca

SEQ ID NO: 1126 - DNA, 10 bp, synthetic construct
  source 1..10: mol_type = other DNA
gaacgttcgg

SEQ ID NO: 1127 - DNA, 10 bp, synthetic construct
  source 1..10: mol_type = other DNA
tggttggaat

SEQ ID NO: 1128 - DNA, 10 bp, synthetic construct
  source 1..10: mol_type = other DNA
aacaagtgag

SEQ ID NO: 1129 - DNA, 10 bp, synthetic construct
  source 1..10: mol_type = other DNA
gttgttgctc

SEQ ID NO: 1130 - DNA, 10 bp, synthetic construct
  source 1..10: mol_type = other DNA
tgacgcaact

SEQ ID NO: 1131 - DNA, 10 bp, synthetic construct
  source 1..10: mol_type = other DNA
ccgtcactga

SEQ ID NO: 1132 - DNA, 10 bp, synthetic construct
  source 1..10: mol_type = other DNA
tgttattccg

SEQ ID NO: 1133 - DNA, 10 bp, synthetic construct
  source 1..10: mol_type = other DNA
ctactcaaga

SEQ ID NO: 1134 - DNA, 10 bp, synthetic construct
  source 1..10: mol_type = other DNA
acgagacgtc

SEQ ID NO: 1135 - DNA, 10 bp, synthetic construct
  source 1..10: mol_type = other DNA
gacgcggtat

SEQ ID NO: 1136 - DNA, 10 bp, synthetic construct
  source 1..10: mol_type = other DNA
gatgacgtta

SEQ ID NO: 1137 - DNA, 10 bp, synthetic construct
  source 1..10: mol_type = other DNA
agccgatacc

SEQ ID NO: 1138 - DNA, 10 bp, synthetic construct
  source 1..10: mol_type = other DNA
ttatctcgag

SEQ ID NO: 1139 - DNA, 10 bp, synthetic construct
  source 1..10: mol_type = other DNA
ccgatgacgt

SEQ ID NO: 1140 - DNA, 10 bp, synthetic construct
  source 1..10: mol_type = other DNA
gtgagttcgc

SEQ ID NO: 1141 - DNA, 10 bp, synthetic construct
  source 1..10: mol_type = other DNA
acacaacatg

SEQ ID NO: 1142 - DNA, 10 bp, synthetic construct
  source 1..10: mol_type = other DNA
cgtgcgatca

SEQ ID NO: 1143 - DNA, 10 bp, synthetic construct
  source 1..10: mol_type = other DNA
tacttcggat

SEQ ID NO: 1144 - DNA, 10 bp, synthetic construct
  source 1..10: mol_type = other DNA
ctatcggtgt

SEQ ID NO: 1145 - DNA, 10 bp, synthetic construct
  source 1..10: mol_type = other DNA
gacattcaag

SEQ ID NO: 1146 - DNA, 10 bp, synthetic construct
  source 1..10: mol_type = other DNA
agtgacacca

SEQ ID NO: 1147 - DNA, 10 bp, synthetic construct
  source 1..10: mol_type = other DNA
tcgcgatgtc

SEQ ID NO: 1148 - DNA, 10 bp, synthetic construct
  source 1..10: mol_type = other DNA
cgagtcagtc

SEQ ID NO: 1149 - DNA, 10 bp, synthetic construct
  source 1..10: mol_type = other DNA
ttgccagtga

SEQ ID NO: 1150 - DNA, 10 bp, synthetic construct
  source 1..10: mol_type = other DNA
aacaagcact

SEQ ID NO: 1151 - DNA, 10 bp, synthetic construct
  source 1..10: mol_type = other DNA
gcttgttcag

SEQ ID NO: 1152 - DNA, 10 bp, synthetic construct
  source 1..10: mol_type = other DNA
tagtgatgtg

SEQ ID NO: 1153 - DNA, 10 bp, synthetic construct
  source 1..10: mol_type = other DNA
cgtgtgacat

SEQ ID NO: 1154 - DNA, 10 bp, synthetic construct
  source 1..10: mol_type = other DNA
atccaccacc

SEQ ID NO: 1155 - DNA, 10 bp, synthetic construct
  source 1..10: mol_type = other DNA
gcaactgtga

SEQ ID NO: 1156 - DNA, 10 bp, synthetic construct
  source 1..10: mol_type = other DNA
atccaccggt

SEQ ID NO: 1157 - DNA, 10 bp, synthetic construct
  source 1..10: mol_type = other DNA
ccgtcattac

SEQ ID NO: 1158 - DNA, 10 bp, synthetic construct
  source 1..10: mol_type = other DNA
tgagtggcta

SEQ ID NO: 1159 - DNA, 10 bp, synthetic construct
  source 1..10: mol_type = other DNA
gatagtaacg

(19) United States
(12) Patent Application Publication
(10) Pub. No.: US 2026/0218167 A1
(43) Pub. Date: Jul. 30, 2026

(54) METHOD FOR CONSTRUCTING A DNA LIBRARY BASED ON AN MGI PLATFORM AND APPLICATIONS THEREOF

(71) Applicant: Nanodigmbio (Nanjing) Biotechnology Co., LTD., Jiangsu (CN)

(72) Inventors: Yugang HU, Jiangsu (CN); Yan QU, Jiangsu (CN); Biao WANG, Jiangsu (CN); Tong LI, Jiangsu (CN); Qiang WU, Jiangsu (CN)

(73) Assignee: Nanodigmbio (Nanjing) Biotechnology Co., LTD., Jiangsu (CN)

(21) Appl. No.: 19/299,633

(22) Filed: Aug. 14, 2025

(30) Foreign Application Priority Data
Jan. 26, 2025 (CN) 202510124651.X

(51) Int. Cl. C12N 15/10; C12Q 1/6806; C12Q 1/6855; C12Q 1/6869

(52) U.S. Cl. CPC C12N 15/1065; C12Q 1/6806; C12Q 1/6855; C12Q 1/6869

(57) ABSTRACT

Provided in the present application is a method for constructing a DNA library based on an MGI platform and applications thereof. The method includes: ligating a bubble adapter to a target sample to obtain a ligation product; and performing library construction on the ligation product utilizing an amplification primer pair, obtaining an MGI platform based amplified library with dual indexes, where the amplification primer pair has the dual indexes and includes a 5′ end library index and a 3′ end library index; and the 5′ end library index is selected from indexes corresponding to any one of groups of 4-base-balanced phosphorylation ends in Table 1 or Table 2, and the 3′ end library index is selected from indexes corresponding to fixedly-matched non-phosphorylation ends corresponding to the 5′ end library index group in Table 1 or Table 2.

### CROSS-REFERENCE TO RELATED APPLICATION(S)

This application claims priority to Chinese Patent Application No. 202510124651.X filed on Jan. 26, 2025, the disclosure of which is hereby incorporated by reference in its entirety as part of this application.

### SEQUENCE LISTING

The present application includes a sequence listing submitted electronically in XML format, which is hereby incorporated by reference in its entirety. The XML file is named 45539_SequenceListing.xml, created on Aug. 1, 2025, with a file size of 1,012,062 bytes. The sequence listing contains 1159 sequences numbered SEQ ID NOs: 1 to 1159, which is substantially the same as that disclosed in the Chinese patent application No. 202510124651X. The sequence listing does not contain any new content.

### FIELD

The present disclosure relates to the field of DNA library construction, and specifically, to a method for constructing a DNA library based on an MGI platform and applications thereof.

### BACKGROUND

With the booming development of a high-throughput sequencing technology, high-throughput sequencers have also ushered in a new developmental upsurge. At present, the sequencers on the market are mainly categorized into Illumina sequencers, MGI sequencers, and other brands of sequencers. The Illumina sequencers and the MGI sequencers account for more than half of the sequencing market. In recent years, due to various factors, the market for the MGI sequencers in our country has ushered in a great increase. An added market share of the sequencers equals or even far exceeds an added market share of the Illumina sequencers. The wide application and development of the MGI sequencers have given sufficient impetus to studies related to sequencing on an MGI platform.

The design and use of sequencing adapters are essential links during sequencing. Currently, the MGI sequencing platform mainly uses software App-A to convert a Y-type adapter used in a library construction solution of an Illumina platform into a bubble adapter structure suitable for the MGI sequencing platform. The lack of independence in the development and use of adapters for the MGI sequencing platform has always been limited by the Illumina platform, not facilitating development of the MGI sequencing platform.

Although, in recent years, the MGI platform has made new explorations and innovations in terms of sequencing adapters, there are still some problems remaining. For example, in Patent CN111910258B, Nanodigmbio optimized a solution of balancing unique dual indexes in groups of 4 of MGI, solved the problem of easy occurrence of sample crosstalk when the MGI platform used a single index to mark a library, and developed corresponding MGI sequencing index products, that is, an MGISEQ Dual Indexes solution (for MGI). During market testing and usage, the inventor found that when the base composition includes a single base at a proportion of at least 12.5% (as required by the MGI platform sequencing, which mandates that no single base in the base composition falls below 12.5%), the product's metrics were optimally effective. However, if there are a large number of samples (e.g., 384 samples) are loaded at the same time, the data output of index primer combinations for some samples in the Patent CN111910258B is not ideal. During practical application, base imbalance can result in reduced quality of sequencing data and increased sequencing costs.

Therefore, based on the above background, it is essential to develop index adapters to be applied to the MGI platform and can meet the simultaneously loading of a large number of samples.

### SUMMARY

The present disclosure is mainly intended to provide a method for constructing a DNA library based on an MGI platform and applications thereof, so as to solve the problem of unsatisfactory data output when a large number of samples are loaded on an MGI platform in the prior art.

In order to implement the above objective, an aspect of the present disclosure provides a method for constructing a DNA library based on an MGI platform. The above method includes the following operations.

A bubble adapter is ligated to a target sample to obtain a ligation product.

Library construction is performed on the ligation product utilizing an amplification primer pair, obtaining an MGI platform based amplified library with dual indexes.

The amplification primer pair has the dual indexes and includes a 5′ end library index and a 3′ end library index.

The 5′ end library index is selected from indexes corresponding to any one of groups of 4-base-balanced phosphorylation ends in Table 1 or Table 2, and the above 3′ end library index is selected from indexes corresponding to fixedly-matched non-phosphorylation ends corresponding to the above 5′ end library index group in Table 1 or Table 2.

The above 4-base-balance refers to balance of index sequences in groups of 4, at each position from the first to the tenth in the index sequence, there is one base of A, one base of T, one base of C, and one base of G. The above bubble adapter comprises a first adapter sequence and a second adapter sequence, the above first adapter sequence is SEQ ID NO: 769, and the above second adapter sequence is SEQ ID NO: 770.

Each of the above amplification primer pairs further includes a 5′ end universal amplification sequence and a 3′ end universal amplification sequence, the above 5′ end universal amplification sequence includes a universal sequence located upstream of the above 5′ end library index and a universal sequence located downstream of the above 5′ end library index, and the above 3′ end universal amplification sequence comprises a universal sequence located upstream of the above 3′ end library index and a universal sequence located downstream of the above 3′ end library index. The universal sequence located upstream of the above 5′ end library index is SEQ ID NO: 773, the universal sequence located downstream of the above 5′ end library index is SEQ ID NO: 774; the universal sequence located upstream of the above 3′ end library index is SEQ ID NO: 775, and the universal sequence located downstream of the above 3′ end library index is SEQ ID NO: 771, and is used in combination with the first adapter sequence shown in SEQ ID NO: 769 and the second adapter sequence shown in SEQ ID NO: 770.

TABLE 1

| | | | Index | | Index | | | | Index | | Index |
|---|---|---|---|---|---|---|---|---|---|---|---|
| | | | corresponding | | corresponding | | | | corresponding | | corresponding |
| | | | to | | to non- | | | | to | | to non- |
| Balanced | | SEQ ID | phosphorylation | SEQ ID | phosphorylation | Balanced | | SEQ ID | phosphorylation | SEQ ID | phosphorylation |
| combination | Number | NO: | end | NO: | end | combination | Number | NO: | end | NO: | end |
| Group | M1- | 776 | TCACATT | 1159 | GATAGTAAC | Group | M1- | 968 | AGCTACT | 967 | ATGTTCAT |
| 1 | 001 | | GCT | | G | 49 | 193 | | CTG | | CC |
| | M1- | 777 | AATGGCG | 1158 | TGAGTGGCT | | M1- | 969 | CAACGT | 966 | TGACCACC |
| | 002 | | CTC | | A | | 194 | | GAGT | | AG |
| | M1- | 778 | GTCTCAA | 1157 | CCGTCATTA | | M1- | 970 | TCGATG | 965 | CACGGTTG |
| | 003 | | TGA | | C | | 195 | | CTAA | | GT |
| | M1- | 779 | CGGATGC | 1156 | ATCCACCGG | | M1- | 971 | GTTGCA | 964 | GCTAAGGA |
| | 004 | | AAG | | T | | 196 | | AGCC | | TA |
| Group | M1- | 780 | TCGCTTA | 1155 | GCAACTGT | Group | M1- | 972 | CGACATG | 963 | TCTGAAGA |
| 2 | 005 | | AGC | | GA | 50 | 197 | | TGT | | CA |
| | M1- | 781 | CGAGGC | 1154 | ATCCACCA | | M1- | 973 | GATGCGC | 962 | CACTGCAG |
| | 006 | | TTAG | | CC | | 198 | | ATA | | AG |
| | M1- | 782 | GTCTAAG | 1153 | CGTGTGAC | | M1- | 974 | TCGTGAT | 961 | GTAATGTCG |
| | 007 | | GCT | | AT | | 199 | | CAG | | T |
| | M1- | 783 | AATACGC | 1152 | TAGTGATG | | M1- | 975 | ATCATCA | 960 | AGGCCTCTT |
| | 008 | | CTA | | TG | | 200 | | GCC | | C |
| Group | M1- | 784 | AAGCCTA | 1151 | GCTTGTTCA | Group | M1- | 976 | TCAATG | 959 | TAGCAAGT |
| 3 | 009 | | TTG | | G | 51 | 201 | | GCGG | | TG |
| | M1- | 785 | CGCTACT | 1150 | AACAAGCA | | M1- | 977 | CAGTAA | 958 | CGCTTCTC |
| | 010 | | GCA | | CT | | 202 | | CTCT | | CT |
| | M1- | 786 | TCAAGAG | 1149 | TTGCCAGTG | | M1- | 978 | GTTGCC | 957 | ACAAGTAA |
| | 011 | | CAT | | A | | 203 | | TGAC | | GC |
| | M1- | 787 | GTTGTGC | 1148 | CGAGTCAGT | | M1- | 979 | AGCCGT | 956 | GTTGCGCG |
| | 012 | | AGC | | C | | 204 | | AATA | | AA |
| Group | M1- | 788 | AGACAG | 1147 | TCGCGATG | Group | M1- | 980 | CTAATAG | 955 | AACATCGT |
| 4 | 013 | | GAAT | | TC | 52 | 205 | | GCT | | AG |
| | M1- | 789 | CCTTGCC | 1146 | AGTGACAC | | M1- | 981 | TCTCCTC | 954 | TGATCGAG |
| | 014 | | GTA | | CA | | 206 | | CAC | | GC |
| | M1- | 790 | GTCATTA | 1145 | GACATTCA | | M1- | 982 | AGCTGC | 953 | GTTGGACA |
| | 015 | | CGG | | AG | | 207 | | ATGA | | TA |
| | M1- | 791 | TAGGCAT | 1144 | CTATCGGT | | M1- | 983 | GAGGAG | 952 | CCGCATTC |
| | 016 | | TCC | | GT | | 208 | | TATG | | CT |
| Group | M1- | 792 | CATATCA | 1143 | TACTTCGG | Group | M1- | 984 | ACCACG | 951 | AGAACCTA |
| 5 | 017 | | TCG | | AT | 53 | 209 | | TAGC | | CA |
| | M1- | 793 | GCACAAC | 1142 | CGTGCGATC | | M1- | 985 | GAATGCA | 950 | TCGTGTCCA |
| | 018 | | AAT | | A | | 210 | | GTA | | T |
| | M1- | 794 | TTGTCGT | 1141 | ACACAACA | | M1- | 986 | TTGGTAG | 949 | CTCGTAGGT |
| | 019 | | GGC | | TG | | 211 | | CCT | | C |
| | M1- | 795 | AGCGGT | 1140 | GTGAGTTC | | M1- | 987 | CGTCATC | 948 | GATCAGATG |
| | 020 | | GCTA | | GC | | 212 | | TAG | | G |
| Group | M1- | 796 | AGCCAG | 1139 | CCGATGAC | Group | M1- | 988 | TCAATGA | 947 | GTGTGTTGC |
| 6 | 021 | | TAGG | | GT | 54 | 213 | | GGT | | G |
| | M1- | 797 | GTAAGTG | 1138 | TTATCTCGA | | M1- | 989 | AGCGAA | 946 | AGTAAGCC |
| | 022 | | TAC | | G | | 214 | | GCTG | | AA |
| | M1- | 798 | TAGTCAC | 1137 | AGCCGATA | | M1- | 990 | CTGTCTT | 945 | CAACTAGT |
| | 023 | | GTT | | CC | | 215 | | AAC | | GT |
| | M1- | 799 | CCTGTCA | 1136 | GATGACGT | | M1- | 991 | GATCGCC | 944 | TCCGCCAAT |
| | 024 | | CCA | | TA | | 216 | | TCA | | C |
| Group | M1- | 800 | ATCGTGG | 1135 | GACGCGGT | Group | M1- | 992 | GTTCCG | 943 | TGGCGATA |
| 7 | 025 | | ATG | | AT | 55 | 217 | | AATG | | CA |
| | M1- | 801 | TGGAGAT | 1134 | ACGAGACG | | M1- | 993 | TACGTAC | 942 | GCTACCAC |
| | 026 | | CGA | | TC | | 218 | | GCA | | GT |
| | M1- | 802 | CCTCACA | 1133 | CTACTCAAG | | M1- | 994 | CGGAGTT | 941 | AACGTTGTT |
| | 027 | | GAT | | A | | 219 | | CAC | | C |
| | M1- | 803 | GAATCTC | 1132 | TGTTATTCC | | M1- | 995 | ACATACG | 940 | CTATAGCG |
| | 028 | | TCC | | G | | 220 | | TGT | | AG |
| Group 8 | M1- | 804 | GCAGACT | 1131 | CCGTCACTG | Group | M1- | 996 | CTGAAGA | 939 | CGCTTGAAT |
| | 029 | | GAC | | A | 56 | 221 | | GAT | | T |
| | M1- | 805 | CTCATTA | 1130 | TGACGCAA | | M1- | 997 | GAAGCCT | 938 | ATGACTGCC |
| | 030 | | ACG | | CT | | 222 | | CCA | | G |
| | M1- | 806 | TGGTGA | 1129 | GTTGTTGC | | M1- | 998 | TGTCTTC | 937 | TAACACCG |
| | 031 | | GCTT | | TC | | 223 | | ATC | | GC |
| | M1- | 807 | AATCCGC | 1128 | AACAAGTG | | M1- | 999 | ACCTGAG | 936 | GCTGGATTA |
| | 032 | | TGA | | AG | | 224 | | TGG | | A |
| Group | M1- | 808 | TCGCATC | 1127 | TGGTTGGA | Group | M1- | 1000 | GTACGTC | 935 | AGACGGTC |
| 9 | 033 | | AAC | | AT | 57 | 225 | | CTT | | AA |
| | M1- | 809 | AGAACA | 1126 | GAACGTTC | | M1- | 1001 | AACTAG | 934 | TAGATCCA |
| | 034 | | GTGA | | GG | | 226 | | GTCA | | CC |
| | M1- | 810 | CATGTCT | 1125 | ATCGCACG | | M1- | 1002 | TGTGTCA | 933 | GCCTCTATG |
| | 035 | | CCT | | CA | | 227 | | GGC | | G |
| | M1- | 811 | GTCTGG | 1124 | CCTAACATT | | M1- | 1003 | CCGACA | 932 | CTTGAAGG |
| | 036 | | AGTG | | C | | 228 | | TAAG | | TT |
| Group | M1- | 812 | GAGGTCT | 1123 | CGTGTGGAT | Group | M1- | 1004 | TCCACAC | 931 | ATAACGGT |
| 10 | 037 | | GTG | | G | 58 | 229 | | GTC | | GG |
| | M1- | 813 | CTATAGA | 1122 | TTACGTCGG | | M1- | 1005 | CGGCAC | 930 | TGCCACTG |
| | 038 | | CGT | | T | | 230 | | ATGA | | TA |
| | M1- | 814 | TGCAGTG | 1121 | ACGAACATA | | M1- | 1006 | GTATGTT | 929 | CATGTACC |
| | 039 | | ACC | | C | | 231 | | CCT | | AC |
| | M1- | 815 | ACTCCAC | 1120 | GACTCATCC | | M1- | 1007 | AATGTG | 928 | GCGTGTAA |
| | 040 | | TAA | | A | | 232 | | GAAG | | CT |
| Group | M1- | 816 | GCGAAG | 1119 | TGCCTGAC | Group | M1- | 1008 | AGACAG | 927 | TGTAACGC |
| 11 | 041 | | TAGG | | TC | 59 | 233 | | ACGT | | TG |
| | M1- | 817 | TGCCTAA | 1118 | CATTATCGC | | M1- | 1009 | TTCTGT | 926 | GCCGTTAT |
| | 042 | | CCT | | T | | 234 | | GGAG | | GC |
| | M1- | 818 | AATGGTC | 1117 | GTAAGCGA | | M1- | 1010 | CAGGTCT | 925 | CTATCACAC |
| | 043 | | TAC | | GG | | 235 | | TCC | | T |
| | M1- | 819 | CTATCCG | 1116 | ACGGCATT | | M1- | 1011 | GCTACAC | 924 | AAGCGGTG |
| | 044 | | GTA | | AA | | 236 | | ATA | | AA |
| Group | M1- | 820 | TACGCTT | 1115 | GACACTGCC | Group | M1- | 1012 | CTAGCG | 923 | GACCTGTC |
| 12 | 045 | | CAG | | A | 60 | 237 | | ACAC | | CT |
| | M1- | 821 | CGGAGCA | 1114 | CCGTAGCAT | | M1- | 1013 | AGGCAT | 922 | AGTAATCG |
| | 046 | | TCT | | G | | 238 | | TACT | | AC |
| | M1- | 822 | GTACTAG | 1113 | ATTCTATGG | | M1- | 1014 | GACATC | 921 | CTGTGCAT |
| | 047 | | ATC | | C | | 239 | | CGGA | | TA |
| | M1- | 823 | ACTTAGC | 1112 | TGAGGCATA | | M1- | 1015 | TCTTGAG | 920 | TCAGCAGA |
| | 048 | | GGA | | T | | 240 | | TTG | | GG |
| Group | M1- | 824 | ATCACTC | 1111 | ATACGCGC | Group | M1- | 1016 | ACAACA | 919 | ACATGAAT |
| 13 | 049 | | CAT | | CA | 61 | 241 | | GAAG | | GC |
| | M1- | 825 | GATCGCA | 1110 | TCTTAGTG | | M1- | 1017 | CGCTTG | 918 | TGCCTCCA |
| | 050 | | GTG | | AC | | 242 | | TGGA | | CT |
| | M1- | 826 | CCGGAAT | 1109 | GAGGTACA | | M1- | 1018 | GTTGGT | 917 | GATACTTG |
| | 051 | | TCC | | TT | | 243 | | CTCC | | TG |
| | M1- | 827 | TGATTGG | 1108 | CGCACTAT | | M1- | 1019 | TAGCAC | 916 | CTGGAGGC |
| | 052 | | AGA | | GG | | 244 | | ACTT | | AA |
| Group | M1- | 828 | CACAAGG | 1107 | TGGCTCGCT | Group | M1- | 1020 | GCTTGC | 915 | TCGCGGAT |
| 14 | 053 | | TCG | | T | 62 | 245 | | AATA | | CA |
| | M1- | 829 | TCTCGCA | 1106 | GTCACTTAA | | M1- | 1021 | TGGACG | 914 | CATTCTCA |
| | 054 | | GGA | | G | | 246 | | TGCT | | TC |
| | M1- | 830 | GTGGTAT | 1105 | CAATAGAGG | | M1- | 1022 | AACGAA | 913 | GTAGACGG |
| | 055 | | CAT | | C | | 247 | | CCGG | | AG |
| | M1- | 831 | AGATCTC | 1104 | ACTGGACTC | | M1- | 1023 | CTACTTG | 912 | AGCATATC |
| | 056 | | ATC | | A | | 248 | | TAC | | GT |
| Group | M1- | 832 | GATGGAG | 1103 | CCGATCATC | Group | M1- | 1024 | CGGTGAG | 911 | TTGTCGGC |
| 15 | 057 | | ATT | | C | 63 | 249 | | TGA | | AC |
| | M1- | 833 | CTCATTCT | 1102 | GTTCGATAA | | M1- | 1025 | GCAATGC | 910 | GACGAACT |
| | 058 | | GC | | G | | 250 | | ATT | | CG |
| | M1- | 834 | TGGCAGA | 1101 | AGCTCGGCT | | M1- | 1026 | TTCCATT | 909 | AGTAGCTA |
| | 059 | | CAA | | T | | 251 | | CAC | | GT |
| | M1- | 835 | ACATCCT | 1100 | TAAGATCGG | | M1- | 1027 | AATGCCA | 908 | CCACTTAGT |
| | 060 | | GCG | | A | | 252 | | GCG | | A |
| Group | M1- | 836 | ACGTCG | 1099 | CCAGTTAAT | Group | M1- | 1028 | TACTCTT | 907 | TAATAAGC |
| 16 | 061 | | CAGA | | C | 64 | 253 | | CTC | | CG |
| | M1- | 837 | TGTATAG | 1098 | GTTCGATT | | M1- | 1029 | AGGAAG | 906 | CGTCGTCA |
| | 062 | | GCT | | GT | | 254 | | GTAA | | AC |
| | M1- | 838 | CAACACA | 1097 | AGGTACGC | | M1- | 1030 | GTTGGA | 905 | ATCGTGAG |
| | 063 | | TTG | | AG | | 255 | | CGGT | | TT |
| | M1- | 839 | GTCGGTT | 1096 | TACACGCGC | | M1- | 1031 | CCACTC | 904 | GCGACCTT |
| | 064 | | CAC | | A | | 256 | | AACG | | GA |
| Group | M1- | 840 | AGCCATA | 1095 | TGCTATGCA | Group | M1- | 1032 | AATGACT | 903 | GAACAGGA |
| 17 | 065 | | AGC | | G | 65 | 257 | | GGT | | CC |
| | M1- | 841 | GTATTCC | 1094 | GTTCGCTGT | | M1- | 1033 | GTGCCA | 902 | TGTGCTCC |
| | 066 | | GAG | | A | | 258 | | CAAC | | TA |
| | M1- | 842 | CATAGGT | 1093 | CCAGTACTG | | M1- | 1034 | CGAATG | 901 | CTCAGCTT |
| | 067 | | TCA | | C | | 259 | | ATCG | | AG |
| | M1- | 843 | TCGGCAG | 1092 | AAGACGAA | | M1- | 1035 | TCCTGT | 900 | ACGTTAAG |
| | 068 | | CTT | | CT | | 260 | | GCTA | | GT |
| Group | M1- | 844 | GTTCGGT | 1091 | AGAACGCG | Group | M1- | 1036 | TGTGAAT | 899 | CGTAGAGT |
| 18 | 069 | | CCT | | TC | 66 | 261 | | TGG | | CA |
| | M1- | 845 | TGGATTG | 1090 | TTCTGCTT | | M1- | 1037 | AACCGGC | 898 | GACCAGTA |
| | 070 | | TAG | | GT | | 262 | | CTT | | AG |
| | M1- | 846 | CCAGAA | 1089 | GCGGATAC | | M1- | 1038 | CTATCCG | 897 | ACGGTTCG |
| | 071 | | CGTC | | AG | | 263 | | ACC | | TC |
| | M1- | 847 | AACTCCA | 1088 | CATCTAGAC | | M1- | 1039 | GCGATTA | 896 | TTATCCACG |
| | 072 | | AGA | | A | | 264 | | GAA | | T |
| Group | M1- | 848 | CGTACAC | 1087 | AACAAGGC | Group | M1- | 1040 | CGTAAC | 895 | GCAGGATG |
| 19 | 073 | | TGG | | TT | 67 | 265 | | CGCA | | GT |
| | M1- | 849 | GCACACA | 1086 | TGATGTCGA | | M1- | 1041 | GACTGA | 894 | AGTATCAC |
| | 074 | | GCA | | A | | 266 | | TAAC | | TC |
| | M1- | 850 | ATGGTGT | 1085 | GCTCCATTC | | M1- | 1042 | ATGCTTA | 893 | TTGTCTGT |
| | 075 | | ATC | | C | | 267 | | CTG | | AG |
| | M1- | 851 | TACTGTG | 1084 | CTGGTCAAG | | M1- | 1043 | TCAGCGG | 892 | CACCAGCA |
| | 076 | | CAT | | G | | 268 | | TGT | | CA |
| Group | M1- | 852 | CGGCAAT | 1083 | GATATGCT | Group | M1- | 1044 | ACATAAC | 891 | CTTGTATA |
| 20 | 077 | | CAG | | GA | 68 | 269 | | ACC | | CC |
| | M1- | 853 | GTAGTTC | 1082 | CTGGATTAC | | M1- | 1045 | CACCTG | 890 | TGCTCTCT |
| | 078 | | GGA | | T | | 270 | | AGGT | | GG |
| | M1- | 854 | TACAGGA | 1081 | AGACGAGG | | M1- | 1046 | GTGACT | 889 | GAACGGAC |
| | 079 | | ACT | | TC | | 271 | | GTAA | | TA |
| | M1- | 855 | ACTTCCG | 1080 | TCCTCCACA | | M1- | 1047 | TGTGGC | 888 | ACGAACGG |
| | 080 | | TTC | | G | | 272 | | TCTG | | AT |
| Group | M1- | 856 | TGCTCCA | 1079 | CAAGACTC | Group | M1- | 1048 | CTCAGA | 887 | GCACCGCT |
| 21 | 081 | | CGA | | AA | 69 | 273 | | CTCT | | AT |
| | M1- | 857 | AATCAAG | 1078 | GTGTCTGA | | M1- | 1049 | ACACATG | 886 | TTGAGCGA |
| | 082 | | GTC | | CC | | 274 | | CTA | | CG |
| | M1- | 858 | GTAGGTC | 1077 | ACCAGACG | | M1- | 1050 | TGTGCCT | 885 | CGCTTAAGT |
| | 083 | | AAT | | GT | | 275 | | AAG | | A |
| | M1- | 859 | CCGATGT | 1076 | TGTCTGATT | | M1- | 1051 | GAGTTGA | 884 | AATGATTCG |
| | 084 | | TCG | | G | | 276 | | GGC | | C |
| Group | M1- | 860 | GACGTG | 1075 | AGCGTATTA | Group | M1- | 1052 | AGCCGTT | 883 | TGGCACTG |
| 22 | 085 | | TGCA | | C | 70 | 277 | | CTC | | AT |
| | M1- | 861 | TCGCCAC | 1074 | CTGTAGCG | | M1- | 1053 | TTGTTGG | 882 | GTCACGAC |
| | 086 | | TTC | | CT | | 278 | | TCT | | TG |
| | M1- | 862 | ATAAGCA | 1073 | TCAAGCAC | | M1- | 1054 | CAAGAC | 881 | CATTGAGA |
| | 087 | | CGT | | GG | | 279 | | AAGA | | GC |
| | M1- | 863 | CGTTATG | 1072 | GATCCTGA | | M1- | 1055 | GCTACAC | 880 | ACAGTTCTC |
| | 088 | | AAG | | TA | | 280 | | GAG | | A |
| Group | M1- | 864 | AATAGAG | 1071 | TGCGCAGA | Group | M1- | 1056 | GTGACG | 879 | TGGTATTC |
| 23 | 089 | | CCA | | AG | 71 | 281 | | CGAT | | TC |
| | M1- | 865 | GTACCTC | 1070 | GCTTGGTC | | M1- | 1057 | AACCTCT | 878 | ATAAGCGG |
| | 090 | | GAC | | GA | | 282 | | CTG | | AG |
| | M1- | 866 | CCGGTG | 1069 | CTAATCCGT | | M1- | 1058 | TCTTGAG | 877 | GCCGTGAA |
| | 091 | | ATTG | | T | | 283 | | AGA | | CA |
| | M1- | 867 | TGCTACT | 1068 | AAGCATATC | | M1- | 1059 | CGAGAT | 876 | CATCCACT |
| | 092 | | AGT | | C | | 284 | | ATCC | | GT |
| Group | M1- | 868 | GATCCG | 1067 | TAGCCTAA | Group | M1- | 1060 | GAAGGAT | 875 | TCTCTCAAC |
| 24 | 093 | | GACT | | GA | 72 | 285 | | TCA | | A |
| | M1- | 869 | TTAGGCA | 1066 | GCCTGACG | | M1- | 1061 | TCGCCTG | 874 | AGCGGTGG |
| | 094 | | CAA | | TT | | 286 | | GTT | | AT |
| | M1- | 870 | AGCATTC | 1065 | CTTATGGC | | M1- | 1062 | AGTAACA | 873 | CAGACATCT |
| | 095 | | TTG | | CG | | 287 | | CGG | | G |
| | M1- | 871 | CCGTAAT | 1064 | AGAGACTT | | M1- | 1063 | CTCTTGC | 872 | GTATAGCTG |
| | 096 | | GGC | | AC | | 288 | | AAC | | C |
| Group | M1- | 872 | GTATAGC | 1063 | CTCTTGCAA | Group | M1- | 1064 | AGAGAC | 871 | CCGTAATG |
| 25 | 097 | | TGC | | C | 73 | 289 | | TTAC | | GC |
| | M1- | 873 | CAGACAT | 1062 | AGTAACAC | | M1- | 1065 | CTTATGG | 870 | AGCATTCT |
| | 098 | | CTG | | GG | | 290 | | CCG | | TG |
| | M1- | 874 | AGCGGTG | 1061 | TCGCCTGGT | | M1- | 1066 | GCCTGA | 869 | TTAGGCAC |
| | 099 | | GAT | | T | | 291 | | CGTT | | AA |
| | M1- | 875 | TCTCTCA | 1060 | GAAGGATT | | M1- | 1067 | TAGCCTA | 868 | GATCCGGA |
| | 100 | | ACA | | CA | | 292 | | AGA | | CT |
| Group | M1- | 876 | CATCCAC | 1059 | CGAGATAT | Group | M1- | 1068 | AAGCATA | 867 | TGCTACTA |
| 26 | 101 | | TGT | | CC | 74 | 293 | | TCC | | GT |
| | M1- | 877 | GCCGTGA | 1058 | TCTTGAGAG | | M1- | 1069 | CTAATCC | 866 | CCGGTGAT |
| | 102 | | ACA | | A | | 294 | | GTT | | TG |
| | M1- | 878 | ATAAGCG | 1057 | AACCTCTCT | | M1- | 1070 | GCTTGG | 865 | GTACCTCG |
| | 103 | | GAG | | G | | 295 | | TCGA | | AC |
| | M1- | 879 | TGGTATT | 1056 | GTGACGCG | | M1- | 1071 | TGCGCAG | 864 | AATAGAGC |
| | 104 | | CTC | | AT | | 296 | | AAG | | CA |
| Group | M1- | 880 | ACAGTTC | 1055 | GCTACACG | Group | M1- | 1072 | GATCCT | 863 | CGTTATGA |
| 27 | 105 | | TCA | | AG | 75 | 297 | | GATA | | AG |
| | M1- | 881 | CATTGAG | 1054 | CAAGACAA | | M1- | 1073 | TCAAGC | 862 | ATAAGCAC |
| | 106 | | AGC | | GA | | 298 | | ACGG | | GT |
| | M1- | 882 | GTCACG | 1053 | TTGTTGGT | | M1- | 1074 | CTGTAG | 861 | TCGCCACT |
| | 107 | | ACTG | | CT | | 299 | | CGCT | | TC |
| | M1- | 883 | TGGCACT | 1052 | AGCCGTTC | | M1- | 1075 | AGCGTAT | 860 | GACGTGTG |
| | 108 | | GAT | | TC | | 300 | | TAC | | CA |
| Group | M1- | 884 | AATGATT | 1051 | GAGTTGAG | Group | M1- | 1076 | TGTCTG | 859 | CCGATGTT |
| 28 | 109 | | CGC | | GC | 76 | 301 | | ATTG | | CG |
| | M1- | 885 | CGCTTAA | 1050 | TGTGCCTA | | M1- | 1077 | ACCAGA | 858 | GTAGGTCA |
| | 110 | | GTA | | AG | | 302 | | CGGT | | AT |
| | M1- | 886 | TTGAGC | 1049 | ACACATGC | | M1- | 1078 | GTGTCT | 857 | AATCAAGG |
| | 111 | | GACG | | TA | | 303 | | GACC | | TC |
| | M1- | 887 | GCACCGC | 1048 | CTCAGACTC | | M1- | 1079 | CAAGAC | 856 | TGCTCCAC |
| | 112 | | TAT | | T | | 304 | | TCAA | | GA |
| Group | M1- | 888 | ACGAAC | 1047 | TGTGGCTC | Group | M1- | 1080 | TCCTCCA | 855 | ACTTCCGTT |
| 29 | 113 | | GGAT | | TG | 77 | 305 | | CAG | | C |
| | M1- | 889 | GAACGG | 1046 | GTGACTGT | | M1- | 1081 | AGACGA | 854 | TACAGGAA |
| | 114 | | ACTA | | AA | | 306 | | GGTC | | CT |
| | M1- | 890 | TGCTCTC | 1045 | CACCTGAG | | M1- | 1082 | CTGGATT | 853 | GTAGTTCG |
| | 115 | | TGG | | GT | | 307 | | ACT | | GA |
| | M1- | 891 | CTTGTAT | 1044 | ACATAACA | | M1- | 1083 | GATATGC | 852 | CGGCAATC |
| | 116 | | ACC | | CC | | 308 | | TGA | | AG |
| Group | M1- | 892 | CACCAGC | 1043 | TCAGCGGTG | Group | M1- | 1084 | CTGGTCA | 851 | TACTGTGCA |
| 30 | 117 | | ACA | | T | 78 | 309 | | AGG | | T |
| | M1- | 893 | TTGTCTG | 1042 | ATGCTTACT | | M1- | 1085 | GCTCCAT | 850 | ATGGTGTAT |
| | 118 | | TAG | | G | | 310 | | TCC | | C |
| | M1- | 894 | AGTATCA | 1041 | GACTGATAA | | M1- | 1086 | TGATGTC | 849 | GCACACAG |
| | 119 | | CTC | | C | | 311 | | GAA | | CA |
| | M1- | 895 | GCAGGA | 1040 | CGTAACCG | | M1- | 1087 | AACAAG | 848 | CGTACACT |
| | 120 | | TGGT | | CA | | 312 | | GCTT | | GG |
| Group | M1- | 896 | TTATCCA | 1039 | GCGATTAG | Group | M1- | 1088 | CATCTAG | 847 | AACTCCAA |
| 31 | 121 | | CGT | | AA | 79 | 313 | | ACA | | GA |
| | M1- | 897 | ACGGTTC | 1038 | CTATCCGA | | M1- | 1089 | GCGGAT | 846 | CCAGAACG |
| | 122 | | GTC | | CC | | 314 | | ACAG | | TC |
| | M1- | 898 | GACCAG | 1037 | AACCGGCC | | M1- | 1090 | TTCTGCT | 845 | TGGATTGT |
| | 123 | | TAAG | | TT | | 315 | | TGT | | AG |
| | M1- | 899 | CGTAGA | 1036 | TGTGAATT | | M1- | 1091 | AGAACG | 844 | GTTCGGTC |
| | 124 | | GTCA | | GG | | 316 | | CGTC | | CT |
| Group | M1- | 900 | ACGTTAA | 1035 | TCCTGTGCT | Group | M1- | 1092 | AAGACG | 843 | TCGGCAGC |
| 32 | 125 | | GGT | | A | 80 | 317 | | AACT | | TT |
| | M1- | 901 | CTCAGCT | 1034 | CGAATGATC | | M1- | 1093 | CCAGTAC | 842 | CATAGGTTC |
| | 126 | | TAG | | G | | 318 | | TGC | | A |
| | M1- | 902 | TGTGCTC | 1033 | GTGCCACAA | | M1- | 1094 | GTTCGC | 841 | GTATTCCG |
| | 127 | | CTA | | C | | 319 | | TGTA | | AG |
| | M1- | 903 | GAACAGG | 1032 | AATGACTGG | | M1- | 1095 | TGCTATG | 840 | AGCCATAA |
| | 128 | | ACC | | T | | 320 | | CAG | | GC |
| Group | M1- | 904 | GCGACCT | 1031 | CCACTCAAC | Group | M1- | 1096 | TACACGC | 839 | GTCGGTTC |
| 33 | 129 | | TGA | | G | 81 | 321 | | GCA | | AC |
| | M1- | 905 | ATCGTGA | 1030 | GTTGGACG | | M1- | 1097 | AGGTACG | 838 | CAACACATT |
| | 130 | | GTT | | GT | | 322 | | CAG | | G |
| | M1- | 906 | CGTCGTC | 1029 | AGGAAGGT | | M1- | 1098 | GTTCGAT | 837 | TGTATAGGC |
| | 131 | | AAC | | AA | | 323 | | TGT | | T |
| | M1- | 907 | TAATAAG | 1028 | TACTCTTCT | | M1- | 1099 | CCAGTTA | 836 | ACGTCGCA |
| | 132 | | CCG | | C | | 324 | | ATC | | GA |
| Group | M1- | 908 | CCACTTA | 1027 | AATGCCAG | Group | M1- | 1100 | TAAGATC | 835 | ACATCCTG |
| 34 | 133 | | GTA | | CG | 82 | 325 | | GGA | | CG |
| | M1- | 909 | AGTAGCT | 1026 | TTCCATTCA | | M1- | 1101 | AGCTCG | 834 | TGGCAGAC |
| | 134 | | AGT | | C | | 326 | | GCTT | | AA |
| | M1- | 910 | GACGAA | 1025 | GCAATGCA | | M1- | 1102 | GTTCGAT | 833 | CTCATTCT |
| | 135 | | CTCG | | TT | | 327 | | AAG | | GC |
| | M1- | 911 | TTGTCGG | 1024 | CGGTGAGT | | M1- | 1103 | CCGATC | 832 | GATGGAGA |
| | 136 | | CAC | | GA | | 328 | | ATCC | | TT |
| Group | M1- | 912 | AGCATAT | 1023 | CTACTTGTA | Group | M1- | 1104 | ACTGGA | 831 | AGATCTCA |
| 35 | 137 | | CGT | | C | 83 | 329 | | CTCA | | TC |
| | M1- | 913 | GTAGAC | 1022 | AACGAACC | | M1- | 1105 | CAATAGA | 830 | GTGGTATC |
| | 138 | | GGAG | | GG | | 330 | | GGC | | AT |
| | M1- | 914 | CATTCTC | 1021 | TGGACGTG | | M1- | 1106 | GTCACT | 829 | TCTCGCAG |
| | 139 | | ATC | | CT | | 331 | | TAAG | | GA |
| | M1- | 915 | TCGCGG | 1020 | GCTTGCAA | | M1- | 1107 | TGGCTC | 828 | CACAAGGT |
| | 140 | | ATCA | | TA | | 332 | | GCTT | | CG |
| Group | M1- | 916 | CTGGAG | 1019 | TAGCACAC | Group | M1- | 1108 | CGCACTA | 827 | TGATTGGA |
| 36 | 141 | | GCAA | | TT | 84 | 333 | | TGG | | GA |
| | M1- | 917 | GATACTT | 1018 | GTTGGTCT | | M1- | 1109 | GAGGTAC | 826 | CCGGAATTC |
| | 142 | | GTG | | CC | | 334 | | ATT | | C |
| | M1- | 918 | TGCCTCC | 1017 | CGCTTGTG | | M1- | 1110 | TCTTAGT | 825 | GATCGCAG |
| | 143 | | ACT | | GA | | 335 | | GAC | | TG |
| | M1- | 919 | ACATGAA | 1016 | ACAACAGA | | M1- | 1111 | ATACGCG | 824 | ATCACTCCA |
| | 144 | | TGC | | AG | | 336 | | CCA | | T |
| Group | M1- | 920 | TCAGCA | 1015 | TCTTGAGT | Group | M1- | 1112 | TGAGGC | 823 | ACTTAGCG |
| 37 | 145 | | GAGG | | TG | 85 | 337 | | ATAT | | GA |
| | M1- | 921 | CTGTGCA | 1014 | GACATCCG | | M1- | 1113 | ATTCTAT | 822 | GTACTAGA |
| | 146 | | TTA | | GA | | 338 | | GGC | | TC |
| | M1- | 922 | AGTAATC | 1013 | AGGCATTA | | M1- | 1114 | CCGTAG | 821 | CGGAGCAT |
| | 147 | | GAC | | CT | | 339 | | CATG | | CT |
| | M1- | 923 | GACCTGT | 1012 | CTAGCGAC | | M1- | 1115 | GACACT | 820 | TACGCTTC |
| | 148 | | CCT | | AC | | 340 | | GCCA | | AG |
| Group | M1- | 924 | AAGCGGT | 1011 | GCTACACAT | Group | M1- | 1116 | ACGGCAT | 819 | CTATCCGGT |
| 38 | 149 | | GAA | | A | 86 | 341 | | TAA | | A |
| | M1- | 925 | CTATCAC | 1010 | CAGGTCTT | | M1- | 1117 | GTAAGCG | 818 | AATGGTCTA |
| | 150 | | ACT | | CC | | 342 | | AGG | | C |
| | M1- | 926 | GCCGTTA | 1009 | TTCTGTGGA | | M1- | 1118 | CATTATC | 817 | TGCCTAACC |
| | 151 | | TGC | | G | | 343 | | GCT | | T |
| | M1- | 927 | TGTAACG | 1008 | AGACAGAC | | M1- | 1119 | TGCCTGA | 816 | GCGAAGTA |
| | 152 | | CTG | | GT | | 344 | | CTC | | GG |
| Group | M1- | 928 | GCGTGTA | 1007 | AATGTGGAA | Group | M1- | 1120 | GACTCAT | 815 | ACTCCACT |
| 39 | 153 | | ACT | | G | 87 | 345 | | CCA | | AA |
| | M1- | 929 | CATGTAC | 1006 | GTATGTTCC | | M1- | 1121 | ACGAAC | 814 | TGCAGTGA |
| | 154 | | CAC | | T | | 346 | | ATAC | | CC |
| | M1- | 930 | TGCCACT | 1005 | CGGCACATG | | M1- | 1122 | TTACGTC | 813 | CTATAGAC |
| | 155 | | GTA | | A | | 347 | | GGT | | GT |
| | M1- | 931 | ATAACGG | 1004 | TCCACACGT | | M1- | 1123 | CGTGTGG | 812 | GAGGTCTG |
| | 156 | | TGG | | C | | 348 | | ATG | | TG |
| Group | M1- | 932 | CTTGAAG | 1003 | CCGACATA | Group | M1- | 1124 | CCTAACA | 811 | GTCTGGAG |
| 40 | 157 | | GTT | | AG | 88 | 349 | | TTC | | TG |
| | M1- | 933 | GCCTCTA | 1002 | TGTGTCAGG | | M1- | 1125 | ATCGCA | 810 | CATGTCTC |
| | 158 | | TGG | | C | | 350 | | CGCA | | CT |
| | M1- | 934 | TAGATCC | 1001 | AACTAGGT | | M1- | 1126 | GAACGTT | 809 | AGAACAGT |
| | 159 | | ACC | | CA | | 351 | | CGG | | GA |
| | M1- | 935 | AGACGG | 1000 | GTACGTCC | | M1- | 1127 | TGGTTG | 808 | TCGCATCA |
| | 160 | | TCAA | | TT | | 352 | | GAAT | | AC |
| Group | M1- | 936 | GCTGGAT | 999 | ACCTGAGTG | Group | M1- | 1128 | AACAAG | 807 | AATCCGCT |
| 41 | 161 | | TAA | | G | 89 | 353 | | TGAG | | GA |
| | M1- | 937 | TAACACC | 998 | TGTCTTCAT | | M1- | 1129 | GTTGTT | 806 | TGGTGAGC |
| | 162 | | GGC | | C | | 354 | | GCTC | | TT |
| | M1- | 938 | ATGACTG | 997 | GAAGCCTC | | M1- | 1130 | TGACGC | 805 | CTCATTAA |
| | 163 | | CCG | | CA | | 355 | | AACT | | CG |
| | M1- | 939 | CGCTTGA | 996 | CTGAAGAG | | M1- | 1131 | CCGTCAC | 804 | GCAGACTG |
| | 164 | | ATT | | AT | | 356 | | TGA | | AC |
| Group | M1- | 940 | CTATAGC | 995 | ACATACGT | Group | M1- | 1132 | TGTTATT | 803 | GAATCTCT |
| 42 | 165 | | GAG | | GT | 90 | 357 | | CCG | | CC |
| | M1- | 941 | AACGTTG | 994 | CGGAGTTCA | | M1- | 1133 | CTACTCA | 802 | CCTCACAG |
| | 166 | | TTC | | C | | 358 | | AGA | | AT |
| | M1- | 942 | GCTACCA | 993 | TACGTACGC | | M1- | 1134 | ACGAGA | 801 | TGGAGATC |
| | 167 | | CGT | | A | | 359 | | CGTC | | GA |
| | M1- | 943 | TGGCGAT | 992 | GTTCCGAA | | M1- | 1135 | GACGCG | 800 | ATCGTGGA |
| | 168 | | ACA | | TG | | 360 | | GTAT | | TG |
| Group | M1- | 944 | TCCGCCA | 991 | GATCGCCT | Group | M1- | 1136 | GATGACG | 799 | CCTGTCACC |
| 43 | 169 | | ATC | | CA | 91 | 361 | | TTA | | A |
| | M1- | 945 | CAACTAG | 990 | CTGTCTTA | | M1- | 1137 | AGCCGAT | 798 | TAGTCACGT |
| | 170 | | TGT | | AC | | 362 | | ACC | | T |
| | M1- | 946 | AGTAAGC | 989 | AGCGAAGC | | M1- | 1138 | TTATCTC | 797 | GTAAGTGTA |
| | 171 | | CAA | | TG | | 363 | | GAG | | C |
| | M1- | 947 | GTGTGTT | 988 | TCAATGAG | | M1- | 1139 | CCGATGA | 796 | AGCCAGTA |
| | 172 | | GCG | | GT | | 364 | | CGT | | GG |
| Group | M1- | 948 | GATCAGA | 987 | CGTCATCTA | Group | M1- | 1140 | GTGAGT | 795 | AGCGGTGC |
| 44 | 173 | | TGG | | G | 92 | 365 | | TCGC | | TA |
| | M1- | 949 | CTCGTAG | 986 | TTGGTAGC | | M1- | 1141 | ACACAA | 794 | TTGTCGTG |
| | 174 | | GTC | | CT | | 366 | | CATG | | GC |
| | M1- | 950 | TCGTGTC | 985 | GAATGCAG | | M1- | 1142 | CGTGCG | 793 | GCACAACA |
| | 175 | | CAT | | TA | | 367 | | ATCA | | AT |
| | M1- | 951 | AGAACCT | 984 | ACCACGTA | | M1- | 1143 | TACTTCG | 792 | CATATCATC |
| | 176 | | ACA | | GC | | 368 | | GAT | | G |
| Group | M1- | 952 | CCGCATT | 983 | GAGGAGTAT | Group | M1- | 1144 | CTATCGG | 791 | TAGGCATTC |
| 45 | 177 | | CCT | | G | 93 | 369 | | TGT | | C |
| | M1- | 953 | GTTGGAC | 982 | AGCTGCATG | | M1- | 1145 | GACATTC | 790 | GTCATTACG |
| | 178 | | ATA | | A | | 370 | | AAG | | G |
| | M1- | 954 | TGATCGA | 981 | TCTCCTCCA | | M1- | 1146 | AGTGACA | 789 | CCTTGCCGT |
| | 179 | | GGC | | C | | 371 | | CCA | | A |
| | M1- | 955 | AACATCG | 980 | CTAATAGGC | | M1- | 1147 | TCGCGAT | 788 | AGACAGGA |
| | 180 | | TAG | | T | | 372 | | GTC | | AT |
| Group | M1- | 956 | GTTGCG | 979 | AGCCGTAA | Group | M1- | 1148 | CGAGTC | 787 | GTTGTGCA |
| 46 | 181 | | CGAA | | TA | 94 | 373 | | AGTC | | GC |
| | M1- | 957 | ACAAGTA | 978 | GTTGCCTG | | M1- | 1149 | TTGCCA | 786 | TCAAGAGC |
| | 182 | | AGC | | AC | | 374 | | GTGA | | AT |
| | M1- | 958 | CGCTTCT | 977 | CAGTAACT | | M1- | 1150 | AACAAG | 785 | CGCTACTGC |
| | 183 | | CCT | | CT | | 375 | | CACT | | A |
| | M1- | 959 | TAGCAAG | 976 | TCAATGGC | | M1- | 1151 | GCTTGTT | 784 | AAGCCTATT |
| | 184 | | TTG | | GG | | 376 | | CAG | | G |
| Group | M1- | 960 | AGGCCTC | 975 | ATCATCAGC | Group | M1- | 1152 | TAGTGAT | 783 | AATACGCC |
| 47 | 185 | | TTC | | C | 95 | 377 | | GTG | | TA |
| | M1- | 961 | GTAATGT | 974 | TCGTGATC | | M1- | 1153 | CGTGTG | 782 | GTCTAAGG |
| | 186 | | CGT | | AG | | 378 | | ACAT | | CT |
| | M1- | 962 | CACTGCA | 973 | GATGCGCAT | | M1- | 1154 | ATCCACC | 781 | CGAGGCTT |
| | 187 | | GAG | | A | | 379 | | ACC | | AG |
| | M1- | 963 | TCTGAAG | 972 | CGACATGTG | | M1- | 1155 | GCAACTG | 780 | TCGCTTAAG |
| | 188 | | ACA | | T | | 380 | | TGA | | C |
| Group | M1- | 964 | GCTAAGG | 971 | GTTGCAAGC | Group | M1- | 1156 | ATCCACC | 779 | CGGATGCA |
| 48 | 189 | | ATA | | C | 96 | 381 | | GGT | | AG |
| | M1- | 965 | CACGGTT | 970 | TCGATGCTA | | M1- | 1157 | CCGTCAT | 778 | GTCTCAATG |
| | 190 | | GGT | | A | | 382 | | TAC | | A |
| | M1- | 966 | TGACCAC | 969 | CAACGTGA | | M1- | 1158 | TGAGTGG | 777 | AATGGCGC |
| | 191 | | CAG | | GT | | 383 | | CTA | | TC |
| | M1- | 967 | ATGTTCAT | 968 | AGCTACTCT | | M1- | 1159 | GATAGTA | 776 | TCACATTGC |
| | 192 | | CC | | G | | 384 | | ACG | | T |

Note:
1. Bold fonts in two columns of “tag corresponding to phosphorylation end” and “tag corresponding to non-phosphorylation end” in the table are indexes that are marked as excellent indexes; normal fonts are indexes that are marked as normal indexes; and the underlined parts are indexes that are marked as unqualified indexes.
2. Bold fonts in the column of “balanced combination” in the table are 4-base balanced combinations that are marked as excellent groups; normal fonts are 4-base balanced combinations that are marked as normal groups; and the underlined parts are 4-base balanced combinations that are marked as unqualified groups.

TABLE 2

| | | | | | | | | | | | Index |
|---|---|---|---|---|---|---|---|---|---|---|---|
| | | | Index | | Index | | | | Index | | corres- |
| | | | corres- | | corres- | | | | corres- | | ponding |
| | | | ponding | | ponding | | | | ponding | | to non- |
| Bal- | | | to phos- | | to non- | Bal- | | | to phos- | | phos- |
| anced | | SEQ | phoryl- | SEQ | phosphoryl- | anced | | SEQ | phoryl- | SEQ | phoryl- |
| combi- | Num- | ID | ation | ID | ation | combin- | Num- | ID | ation | ID | ation |
| nation | ber | NO: | end | NO: | end | ation | ber | NO: | end | NO: | end |
| Group | M2- | 1 | CCGTTGT | 193 | GCTCTGT | Group | M2- | 385 | ATAGGCG | 577 | TCAAGTCG |
| 1 | 001 | | GAG | | GTC | 49 | 193 | | TTA | | CC |
| | M2- | 2 | AATGGTG | 194 | TGGTGCG | | M2- | 386 | TGCCATA | 578 | GTGTACAC |
| | 002 | | AGT | | CAA | | 194 | | CGG | | TA |
| | M2- | 3 | TGCAACC | 195 | AACACAC | | M2- | 387 | CATTCGT | 579 | CGCGTAGT |
| | 003 | | TTC | | TGT | | 195 | | GCC | | GT |
| | M2- | 4 | GTACCAA | 196 | CTAGATA | | M2- | 388 | GCGATA | 580 | AATCCGTA |
| | 004 | | CCA | | ACG | | 196 | | CAAT | | AG |
| Group | M2- | 5 | TGCGTTC | 197 | AGGCCGT | Group | M2- | 389 | TTAACGC | 581 | TGCAGAAT |
| 2 | 005 | | TGC | | TAC | 50 | 197 | | GAC | | CC |
| | M2- | 6 | CTGTCGT | 198 | CTTGGAC | | M2- | 390 | CATGTT | 582 | ACTTAGGA |
| | 006 | | GAT | | CTG | | 198 | | GTCA | | GT |
| | M2- | 7 | ACAAGC | 199 | TCCAATA | | M2- | 391 | AGGCGA | 583 | CTGGTCTC |
| | 007 | | AATG | | GGA | | 199 | | TCTG | | AA |
| | M2- | 8 | GATCAAG | 200 | GAATTCG | | M2- | 392 | GCCTAC | 584 | GAACCTCG |
| | 008 | | CCA | | ACT | | 200 | | AAGT | | TG |
| Group | M2- | 9 | ACTTAGA | 201 | GAATTCGC | Group | M2- | 393 | GAGGTT | 585 | TGGCTACA |
| 3 | 009 | | TCG | | GA | 51 | 201 | | AGTA | | TA |
| | M2- | 10 | TTAGGAG | 202 | CGTGGTC | | M2- | 394 | AGTTCC | 586 | CCATGTAG |
| | 010 | | GAA | | ACT | | 202 | | GCCT | | AG |
| | M2- | 11 | CGCATCC | 203 | TTCACGA | | M2- | 395 | CCACAG | 587 | GTTGACGT |
| | 011 | | ATC | | TAC | | 203 | | CAAG | | CC |
| | M2- | 12 | GAGCCTT | 204 | ACGCAAT | | M2- | 396 | TTCAGAT | 588 | AACACGTC |
| | 012 | | CGT | | GTG | | 204 | | TGC | | GT |
| Group | M2- | 13 | ACTCGCG | 205 | ACAATCC | Group | M2- | 397 | CGGATA | 589 | TAACCACC |
| 4 | 013 | | GTA | | GGC | 52 | 205 | | CACT | | TC |
| | M2- | 14 | CACGAA | 206 | TTGTGGT | | M2- | 398 | GACGAT | 590 | ACGTGCTT |
| | 014 | | CACT | | ATG | | 206 | | AGGA | | AT |
| | M2- | 15 | GTAACTA | 207 | CGCCATGT | | M2- | 399 | TCTCGGT | 591 | GTCGTGGA |
| | 015 | | TGG | | AA | | 207 | | CTG | | GA |
| | M2- | 16 | TGGTTGT | 208 | GATGCAA | | M2- | 400 | ATATCCG | 592 | CGTAATAGC |
| | 016 | | CAC | | CCT | | 208 | | TAC | | G |
| Group | M2- | 17 | AGGTCA | 209 | TATCGTT | Group | M2- | 401 | AGATAGC | 593 | AACCGTGG |
| 5 | 017 | | CTAG | | GTG | 53 | 209 | | CTT | | TA |
| | M2- | 18 | CCTGATA | 210 | ATGACAG | | M2- | 402 | CTTACCA | 594 | GTTGTACTA |
| | 018 | | GCC | | CAC | | 210 | | TGC | | C |
| | M2- | 19 | GACCTG | 211 | CGCTTCA | | M2- | 403 | TCGGTTG | 595 | TCATCGAA |
| | 019 | | GATT | | TGT | | 211 | | GAG | | GG |
| | M2- | 20 | TTAAGCT | 212 | GCAGAGC | | M2- | 404 | GACCGAT | 596 | CGGAACTC |
| | 020 | | CGA | | ACA | | 212 | | ACA | | CT |
| Group | M2- | 21 | CGGTGAA | 213 | CTACGCG | Group | M2- | 405 | AACAACC | 597 | AGTACAAC |
| 6 | 021 | | GTC | | AAG | 54 | 213 | | TCA | | TG |
| | M2- | 22 | GTAACTG | 214 | AAGGTGA | | M2- | 406 | CGGCTGT | 598 | CAAGTCTG |
| | 022 | | CAT | | TTC | | 214 | | AAC | | CA |
| | M2- | 23 | TCTCTGT | 215 | GCTTATTG | | M2- | 407 | GTAGCAG | 599 | GTGCAGCT |
| | 023 | | TGA | | CT | | 215 | | GTG | | AC |
| | M2- | 24 | AACGACC | 216 | TGCACAC | | M2- | 408 | TCTTGTA | 600 | TCCTGTGA |
| | 024 | | ACG | | CGA | | 216 | | CGT | | GT |
| Group | M2- | 25 | GCGAATC | 217 | ACTGGATC | Group | M2- | 409 | ACGTGA | 601 | CACTAATC |
| 7 | 025 | | AGC | | CG | 55 | 217 | | ACAC | | AG |
| | M2- | 26 | AACTTCG | 218 | GAGAACA | | M2- | 410 | CGCACT | 602 | ACTAGCGG |
| | 026 | | GTA | | GAT | | 218 | | GGTA | | CT |
| | M2- | 27 | TGTGGA | 219 | TTACCTC | | M2- | 411 | GTAGAG | 603 | GTAGTTCA |
| | 027 | | ATAG | | AGA | | 219 | | CTCT | | TC |
| | M2- | 28 | CTACCGT | 220 | CGCTTGG | | M2- | 412 | TATCTCT | 604 | TGGCCGAT |
| | 028 | | CCT | | TTC | | 220 | | AGG | | GA |
| Group | M2- | 29 | CCATAAG | 221 | TCACCTCT | Group | M2- | 413 | AACAACT | 605 | ACCATCCG |
| 8 | 029 | | AGG | | CG | 56 | 221 | | TGG | | AA |
| | M2- | 30 | GTCGGTA | 222 | ATCAGCG | | M2- | 414 | CCACCAC | 606 | CATTCGATT |
| | 030 | | GTC | | CTC | | 222 | | CTA | | C |
| | M2- | 31 | TGTCTGC | 223 | CGGTAATG | | M2- | 415 | GTGGTGA | 607 | GTAGGATAG |
| | 031 | | CAT | | AA | | 223 | | GAC | | G |
| | M2- | 32 | AAGACCT | 224 | GATGTGA | | M2- | 416 | TGTTGTG | 608 | TGGCATGCC |
| | 032 | | TCA | | AGT | | 224 | | ACT | | T |
| Group | M2- | 33 | CCGATTC | 225 | TAGTTGG | Group | M2- | 417 | CCATTG | 609 | CGTAGATC |
| 9 | 033 | | GAT | | TAC | 57 | 225 | | AATG | | GC |
| | M2- | 34 | ATTGCAG | 226 | CGAGACA | | M2- | 418 | TTCAATC | 610 | TAAGCGCG |
| | 034 | | CCA | | ACT | | 226 | | GAC | | TG |
| | M2- | 35 | GAATACT | 227 | GTTCCAC | | M2- | 419 | AGGCGC | 611 | GTGCTTGT |
| | 035 | | TGC | | CGA | | 227 | | TTCT | | AT |
| | M2- | 36 | TGCCGG | 228 | ACCAGTT | | M2- | 420 | GATGCA | 612 | ACCTACAA |
| | 036 | | AATG | | GTG | | 228 | | GCGA | | CA |
| Group | M2- | 37 | ATGCGG | 229 | CAAGCAG | Group | M2- | 421 | CACGAT | 613 | AGTCATGA |
| 10 | 037 | | ATCC | | CAG | 58 | 229 | | GTGA | | CA |
| | M2- | 38 | CCTTCAT | 230 | GCGAACT | | M2- | 422 | TGGTCC | 614 | TTGAGCCT |
| | 038 | | GAA | | GGT | | 230 | | AATT | | GG |
| | M2- | 39 | GACGATC | 231 | TGCTGTC | | M2- | 423 | ATTAGGT | 615 | CAAGTATCT |
| | 039 | | ATG | | TTA | | 231 | | GCC | | C |
| | M2- | 40 | TGAATCG | 232 | ATTCTGA | | M2- | 424 | GCACTAC | 616 | GCCTCGAG |
| | 040 | | CGT | | ACC | | 232 | | CAG | | AT |
| Group | M2- | 41 | TACACTC | 233 | ACCAATC | Group | M2- | 425 | AAGTGA | 617 | CACTCGTA |
| 11 | 041 | | GGT | | GTT | 59 | 233 | | TCCA | | GC |
| | M2- | 42 | ATACAGG | 234 | TGTTCAG | | M2- | 426 | GTACATC | 618 | GCTGGTAC |
| | 042 | | ACG | | CAA | | 234 | | TTC | | CT |
| | M2- | 43 | CGTGTCT | 235 | CAGGTGT | | M2- | 427 | TCTGCC | 619 | AGGATCCT |
| | 043 | | CAC | | AGC | | 235 | | GGAT | | AA |
| | M2- | 44 | GCGTGAA | 236 | GTACGCAT | | M2- | 428 | CGCATGA | 620 | TTACAAGG |
| | 044 | | TTA | | CG | | 236 | | AGG | | TG |
| Group | M2- | 45 | TTCGTTC | 237 | CAACACC | Group | M2- | 429 | ACTGAG | 621 | AATGAGGC |
| 12 | 045 | | ACA | | GAG | 60 | 237 | | TAAG | | TA |
| | M2- | 46 | GAGACG | 238 | GTTGCAG | | M2- | 430 | CTCACAG | 622 | GTAAGTCG |
| | 046 | | GTTG | | ATC | | 238 | | CGA | | CG |
| | M2- | 47 | ACACAAT | 239 | TGGTGGT | | M2- | 431 | GAGTTC | 623 | TGCTCCTA |
| | 047 | | GGT | | TGT | | 239 | | AGCT | | AC |
| | M2- | 48 | CGTTGCA | 240 | ACCATTA | | M2- | 432 | TGACGT | 624 | CCGCTAAT |
| | 048 | | CAC | | CCA | | 240 | | CTTC | | GT |
| Group | M2- | 49 | GAACACT | 241 | AGTTGTCT | Group | M2- | 433 | CGCCTGT | 625 | AGTTAGTC |
| 13 | 049 | | ATC | | GC | 61 | 241 | | TGT | | AC |
| | M2- | 50 | TCTGCGG | 242 | CTAGAGG | | M2- | 434 | GAGAATA | 626 | CTCGTTGTC |
| | 050 | | TAA | | ACA | | 242 | | GCA | | G |
| | M2- | 51 | AGGTTAA | 243 | GCGACCT | | M2- | 435 | TTAGCAC | 627 | GCGAGCCA |
| | 051 | | GCG | | CAT | | 243 | | ATC | | TA |
| | M2- | 52 | CTCAGTC | 244 | TACCTAAG | | M2- | 436 | ACTTGCG | 628 | TAACCAAG |
| | 052 | | CGT | | TG | | 244 | | CAG | | GT |
| Group | M2- | 53 | CGCAATG | 245 | GTGTTATC | Group | M2- | 437 | CGAGAA | 629 | CCAGATGT |
| 14 | 053 | | AAC | | TC | 62 | 245 | | TTCA | | GC |
| | M2- | 54 | TTGGCAC | 246 | TCAGACG | | M2- | 438 | GCGTTC | 630 | GATTCGTG |
| | 054 | | TTA | | TCG | | 246 | | GCTT | | AG |
| | M2- | 55 | GCATTCA | 247 | AACCGGA | | M2- | 439 | TTCCGTA | 631 | TTGCTCAC |
| | 055 | | GCG | | AGT | | 247 | | AGC | | CA |
| | M2- | 56 | AATCGGT | 248 | CGTACTCG | | M2- | 440 | AATACGC | 632 | AGCAGACA |
| | 056 | | CGT | | AA | | 248 | | GAG | | TT |
| Group | M2- | 57 | ATATGGC | 249 | ACTCGAA | Group | M2- | 441 | CGTGATT | 633 | TAGCGGAG |
| 15 | 057 | | AGA | | GGT | 63 | 249 | | GGT | | TG |
| | M2- | 58 | CGGACT | 250 | CGCTTGT | | M2- | 442 | ATCATGC | 634 | AGCAACTA |
| | 058 | | GTCT | | AAG | | 250 | | TAC | | GA |
| | M2- | 59 | GATCAAT | 251 | GTAACCG | | M2- | 443 | GCATCCG | 635 | CTAGTTCTC |
| | 059 | | CTG | | CTC | | 251 | | ATG | | T |
| | M2- | 60 | TCCGTCA | 252 | TAGGATCT | | M2- | 444 | TAGCGAA | 636 | GCTTCAGC |
| | 060 | | GAC | | CA | | 252 | | CCA | | AC |
| Group | M2- | 61 | AGAGTGC | 253 | GTCCGATT | Group | M2- | 445 | GATCCT | 637 | CATAATCC |
| 16 | 061 | | ATG | | AC | 64 | 253 | | CTCG | | GG |
| | M2- | 62 | GTTCGCA | 254 | CAGTCGG | | M2- | 446 | TTGAAG | 638 | GTCCGCTG |
| | 062 | | TAC | | ATT | | 254 | | GAAC | | AT |
| | M2- | 63 | TCGAATT | 255 | TGTGATCG | | M2- | 447 | CCATTCT | 639 | AGATCAGT |
| | 063 | | CGT | | CG | | 255 | | GTA | | CC |
| | M2- | 64 | CACTCAG | 256 | ACAATCA | | M2- | 448 | AGCGGA | 640 | TCGGTGAA |
| | 064 | | GCA | | CGA | | 256 | | ACGT | | TA |
| Group | M2- | 65 | CCTTGGT | 257 | TGTGACTA | Group | M2- | 449 | TTCCACC | 641 | CAAGCCTG |
| 17 | 065 | | GCA | | CA | 65 | 257 | | TGG | | GT |
| | M2- | 66 | GAGCATC | 258 | ACACTTG | | M2- | 450 | GCATCTT | 642 | GTCCTTGCC |
| | 066 | | CAT | | CGG | | 258 | | CTT | | A |
| | M2- | 67 | AGAGTCG | 259 | CTGTGGCT | | M2- | 451 | AATGGAG | 643 | TCTAGGATT |
| | 067 | | ATC | | AT | | 259 | | AAC | | G |
| | M2- | 68 | TTCACAA | 260 | GACACAA | | M2- | 452 | CGGATGA | 644 | AGGTAACA |
| | 068 | | TGG | | GTC | | 260 | | GCA | | AC |
| Group | M2- | 69 | GTATCCT | 261 | TCATACTG | Group | M2- | 453 | CATGCC | 645 | CTGGAGGT |
| 18 | 069 | | CAG | | TC | 66 | 261 | | TATG | | CT |
| | M2- | 70 | TACCGAG | 262 | GATACGG | | M2- | 454 | GCGTGGA | 646 | AGCTCTAC |
| | 070 | | TGT | | AGG | | 262 | | CAA | | AA |
| | M2- | 71 | ACTGTGC | 263 | CGGCTAA | | M2- | 455 | AGCAATG | 647 | GATATATGG |
| | 071 | | ACC | | CCA | | 263 | | GCT | | C |
| | M2- | 72 | CGGAATA | 264 | ATCGGTC | | M2- | 456 | TTACTAC | 648 | TCACGCCA |
| | 072 | | GTA | | TAT | | 264 | | TGC | | TG |
| Group | M2- | 73 | GTGTCTA | 265 | GCCTCCTG | Group | M2- | 457 | CGGTAAT | 649 | TGGATCGG |
| 19 | 073 | | CAG | | TT | 67 | 265 | | ATC | | TC |
| | M2- | 74 | TACGTATA | 266 | TATAGGAC | | M2- | 458 | ACAGCC | 650 | ATATAGCC |
| | 074 | | CC | | AC | | 266 | | GGAA | | GG |
| | M2- | 75 | AGTAAGC | 267 | AGAGAAC | | M2- | 459 | GTTCTG | 651 | CCTCCATT |
| | 075 | | GGT | | TGA | | 267 | | CTCG | | AT |
| | M2- | 76 | CCACGCG | 268 | CTGCTTGA | | M2- | 460 | TACAGTA | 652 | GACGGTAA |
| | 076 | | TTA | | CG | | 268 | | CGT | | CA |
| Group | M2- | 77 | AGCTTAT | 269 | CCAACTAC | Group | M2- | 461 | CGTGGA | 653 | CAATTCCT |
| 20 | 077 | | GGC | | CA | 68 | 269 | | GTTG | | CA |
| | M2- | 78 | CCGAATG | 270 | GTGCGGC | | M2- | 462 | GTCTTCT | 654 | GCGAGAAG |
| | 078 | | ATT | | ATT | | 270 | | CGA | | AG |
| | M2- | 79 | GTTGGC | 271 | TGTGAAG | | M2- | 463 | TCACCT | 655 | TTCCAGTA |
| | 079 | | ACCA | | GAG | | 271 | | CAAT | | GT |
| | M2- | 80 | TAACCGC | 272 | AACTTCT | | M2- | 464 | AAGAAG | 656 | AGTGCTGC |
| | 080 | | TAG | | TGC | | 272 | | AGCC | | TC |
| Group | M2- | 81 | AGAAGTA | 273 | TCGTGGA | Group | M2- | 465 | AACGCCG | 657 | TACCGCAC |
| 21 | 081 | | GGA | | ATT | 69 | 273 | | TCA | | AT |
| | M2- | 82 | CTTCCAG | 274 | AATACAG | | M2- | 466 | GCGCTGA | 658 | GCAGTACA |
| | 082 | | TTG | | CGC | | 274 | | ATC | | CG |
| | M2- | 83 | GAGGTG | 275 | GTCCTCC | | M2- | 467 | CTAAGTT | 659 | CGTACGGTT |
| | 083 | | CAAC | | TAA | | 275 | | GGT | | A |
| | M2- | 84 | TCCTACT | 276 | CGAGATT | | M2- | 468 | TGTTAAC | 660 | ATGTATTGG |
| | 084 | | CCT | | GCG | | 276 | | CAG | | C |
| Group | M2- | 85 | TCCGACA | 277 | CCGTAGT | Group | M2- | 469 | CGATACC | 661 | CTGCCTTA |
| 22 | 085 | | GTT | | TGG | 70 | 277 | | TAG | | GT |
| | M2- | 86 | AGTAGTT | 278 | TTAGCTC | | M2- | 470 | GCTATGA | 662 | GATGTGGT |
| | 086 | | ACC | | GTT | | 278 | | CCT | | AC |
| | M2- | 87 | GAATCG | 279 | GATCGCA | | M2- | 471 | TTCGCT | 663 | TCCTACAG |
| | 087 | | GTGG | | CAA | | 279 | | GGTC | | CG |
| | M2- | 88 | CTGCTAC | 280 | AGCATAG | | M2- | 472 | AAGCGA | 664 | AGAAGACC |
| | 088 | | CAA | | ACC | | 280 | | TAGA | | TA |
| Group | M2- | 89 | CGTTGAA | 281 | AGTCGCC | Group | M2- | 473 | TCTGAT | 665 | ACTGGTGC |
| 23 | 089 | | TGG | | ATT | 71 | 281 | | GGAA | | AA |
| | M2- | 90 | ATGGCTT | 282 | GAGTAAT | | M2- | 474 | AGAACG | 666 | CGCCAGAA |
| | 090 | | CTA | | TGG | | 282 | | TCCG | | TC |
| | M2- | 91 | GACATGC | 283 | CTAGCTA | | M2- | 475 | GTCTTC | 667 | GTAATACT |
| | 091 | | ACC | | GCA | | 283 | | CATT | | CG |
| | M2- | 92 | TCACACG | 284 | TCCATGG | | M2- | 476 | CAGCGA | 668 | TAGTCCTG |
| | 092 | | GAT | | CAC | | 284 | | ATGC | | GT |
| Group | M2- | 93 | CCAATAT | 285 | TAGCTTC | Group | M2- | 477 | CGAGCAC | 669 | AGTCTAAG |
| 24 | 093 | | GGT | | CGG | 72 | 285 | | TGT | | GA |
| | M2- | 94 | GTCTCGC | 286 | ACTTACAG | | M2- | 478 | GCTATTG | 670 | GAGGACGA |
| | 094 | | TCA | | CT | | 286 | | ATC | | TC |
| | M2- | 95 | TGGCAC | 287 | CGCAGAT | | M2- | 479 | TAGTACA | 671 | CCATGTTCA |
| | 095 | | GAAC | | ATA | | 287 | | CAG | | T |
| | M2- | 96 | AATGGTA | 288 | GTAGCGG | | M2- | 480 | ATCCGGT | 672 | TTCACGCTC |
| | 096 | | CTG | | TAC | | 288 | | GCA | | G |
| Group | M2- | 97 | GAGGCT | 289 | TCTAATC | Group | M2- | 481 | AACTTG | 673 | GATCTAGC |
| 25 | 097 | | GAAT | | GCA | 73 | 289 | | GTGT | | AC |
| | M2- | 98 | AGTAAGA | 290 | AAGCTAA | | M2- | 482 | GCACGA | 674 | CTGTCGATG |
| | 098 | | GTC | | CAC | | 290 | | AGTG | | G |
| | M2- | 99 | TCCTGAC | 291 | CGAGCGT | | M2- | 483 | CGTAAC | 675 | TCCAGTTA |
| | 099 | | TGA | | AGT | | 291 | | TACC | | CA |
| | M2- | 100 | CTACTCT | 292 | GTCTGCG | | M2- | 484 | TTGGCT | 676 | AGAGACCG |
| | 100 | | CCG | | TTG | | 292 | | CCAA | | TT |
| Group | M2- | 101 | AGGTCTG | 293 | TGAACAA | Group | M2- | 485 | GTTCATC | 677 | GTACAATTC |
| 26 | 101 | | ATT | | CTC | 74 | 293 | | GAG | | G |
| | M2- | 102 | CTTGAGC | 294 | ACTCAGT | | M2- | 486 | TACACAT | 678 | CATTCCGC |
| | 102 | | CAG | | GCT | | 294 | | CGC | | AA |
| | M2- | 103 | GAACGAA | 295 | CTCGTTCA | | M2- | 487 | ACGTGCG | 679 | TGGATGCA |
| | 103 | | GGC | | GG | | 295 | | ACT | | GC |
| | M2- | 104 | TCCATCT | 296 | GAGTGCG | | M2- | 488 | CGAGTG | 680 | ACCGGTAG |
| | 104 | | TCA | | TAA | | 296 | | ATTA | | TT |
| Group | M2- | 105 | CGTCACG | 297 | CGCGACC | Group | M2- | 489 | CAGCTC | 681 | ATGCGTTC |
| 27 | 105 | | CAA | | ATT | 75 | 297 | | CTCT | | CG |
| | M2- | 106 | GCCTGTT | 298 | AATACTGG | | M2- | 490 | GCAGATA | 682 | CCAACACT |
| | 106 | | ATT | | CA | | 298 | | AGC | | AC |
| | M2- | 107 | ATAACAA | 299 | TTACGGA | | M2- | 491 | TGTTGAT | 683 | GACGTCGA |
| | 107 | | GCG | | CGC | | 299 | | GAG | | TA |
| | M2- | 108 | TAGGTGC | 300 | GCGTTAT | | M2- | 492 | ATCACGG | 684 | TGTTAGAG |
| | 108 | | TGC | | TAG | | 300 | | CTA | | GT |
| Group | M2- | 109 | GATCTGA | 301 | TCAGTTC | Group | M2- | 493 | TTCAGA | 685 | CCTCTCAC |
| 28 | 109 | | ACA | | GAT | 76 | 301 | | ACAG | | GT |
| | M2- | 110 | CTCAATG | 302 | CTGAGCA | | M2- | 494 | CAAGCC | 686 | GTCTCAGT |
| | 110 | | TGT | | AGC | | 302 | | GTGA | | CG |
| | M2- | 111 | TCGTGAC | 303 | GACTAAG | | M2- | 495 | AGGTTG | 687 | TAGGAGCA |
| | 111 | | GAC | | TTG | | 303 | | TGTT | | TA |
| | M2- | 112 | AGAGCC | 304 | AGTCCGT | | M2- | 496 | GCTCATC | 688 | AGAAGTTG |
| | 112 | | TCTG | | CCA | | 304 | | ACC | | AC |
| Group | M2- | 113 | CGAAGTA | 305 | GCGGATA | Group | M2- | 497 | CCATACT | 689 | TGGTTCGT |
| 29 | 113 | | CCG | | TTC | 77 | 305 | | CAG | | CG |
| | M2- | 114 | ACCGAAT | 306 | ATACCGC | | M2- | 498 | GTGAGTA | 690 | ACCGCACG |
| | 114 | | AGA | | AAG | | 306 | | TTC | | TT |
| | M2- | 115 | TAGTCGC | 307 | TGTTGCG | | M2- | 499 | TGTGCG | 691 | CTTAAGTC |
| | 115 | | TAT | | CGT | | 307 | | GACA | | AC |
| | M2- | 116 | GTTCTCG | 308 | CACATAT | | M2- | 500 | AACCTA | 692 | GAACGTAA |
| | 116 | | GTC | | GCA | | 308 | | CGGT | | GA |
| Group | M2- | 117 | AACACG | 309 | AGCGTGA | Group | M2- | 501 | TGAACC | 693 | CTCCGCAA |
| 30 | 117 | | AGAA | | CCT | 78 | 309 | | GAAG | | GT |
| | M2- | 118 | CTTCGCT | 310 | GTATAACG | | M2- | 502 | ACCTGG | 694 | GATGTTGC |
| | 118 | | CTC | | AC | | 310 | | CGTA | | AA |
| | M2- | 119 | GCGTATC | 311 | TAGAGCTA | | M2- | 503 | CAGCTAT | 695 | TGATAATGC |
| | 119 | | TCG | | TG | | 311 | | CCT | | G |
| | M2- | 120 | TGAGTAG | 312 | CCTCCTG | | M2- | 504 | GTTGATA | 696 | ACGACGCT |
| | 120 | | AGT | | TGA | | 312 | | TGC | | TC |
| Group | M2- | 121 | TGGAGTT | 313 | CTCAAGG | Group | M2- | 505 | GCCTCAT | 697 | GCACAGAG |
| 31 | 121 | | GCG | | CTC | 79 | 313 | | GAC | | AC |
| | M2- | 122 | GTTGTAA | 314 | GCACCTT | | M2- | 506 | ATACACA | 698 | CTTGCACT |
| | 122 | | CGT | | AGG | | 314 | | CCT | | CA |
| | M2- | 123 | ACCTACC | 315 | TGTGTAC | | M2- | 507 | TATGGTG | 699 | TACTTCGA |
| | 123 | | TAA | | TCT | | 315 | | ATG | | GG |
| | M2- | 124 | CAACCGG | 316 | AAGTGCA | | M2- | 508 | CGGATG | 700 | AGGAGTTC |
| | 124 | | ATC | | GAA | | 316 | | CTGA | | TT |
| Group | M2- | 125 | GATAGCT | 317 | TGCGACG | Group | M2- | 509 | CCGCTTG | 701 | CGGACGTT |
| 32 | 125 | | TCG | | ATA | 80 | 317 | | TTG | | AA |
| | M2- | 126 | CCGCAA | 318 | ATGACGA | | M2- | 510 | TTCTGCT | 702 | ACTCAACG |
| | 126 | | CGAT | | TCG | | 318 | | ACA | | GC |
| | M2- | 127 | AGATCG | 319 | CATTGTT | | M2- | 511 | AGAACA | 703 | GTATGCAAT |
| | 127 | | GATA | | CAC | | 319 | | AGGT | | G |
| | M2- | 128 | TTCGTTA | 320 | GCACTAC | | M2- | 512 | GATGAGC | 704 | TACGTTGCC |
| | 128 | | CGC | | GGT | | 320 | | CAC | | T |
| Group | M2- | 129 | AAGAGA | 321 | GCAACCT | Group | M2- | 513 | ACAAGC | 705 | ACTGGCTA |
| 33 | 129 | | CACA | | GAG | 81 | 321 | | TCTA | | AC |
| | M2- | 130 | CCACTTG | 322 | TAGTAGC | | M2- | 514 | CTGCAG | 706 | CTACTGAT |
| | 130 | | GAT | | ACT | | 322 | | GAAG | | GT |
| | M2- | 131 | GTCTCCT | 323 | AGCCTTA | | M2- | 515 | TGTTCTA | 707 | GACTATGG |
| | 131 | | TGC | | TTC | | 323 | | GCT | | CA |
| | M2- | 132 | TGTGAG | 324 | CTTGGAG | | M2- | 516 | GACGTA | 708 | TGGACACC |
| | 132 | | ACTG | | CGA | | 324 | | CTGC | | TG |
| Group | M2- | 133 | AGGATAG | 325 | AGACGTG | Group | M2- | 517 | GTAAGG | 709 | TCTCACCA |
| 34 | 133 | | CCG | | TTA | 82 | 325 | | TGTT | | GG |
| | M2- | 134 | TCTGCTA | 326 | CCGGTGA | | M2- | 518 | AAGGCT | 710 | AACGTAGG |
| | 134 | | TAC | | GAT | | 326 | | AAGC | | CT |
| | M2- | 135 | GTCTACT | 327 | GTCTCAC | | M2- | 519 | CGCTTC | 711 | CGATCTTC |
| | 135 | | GGT | | AGC | | 327 | | CTAA | | TC |
| | M2- | 136 | CAACGG | 328 | TATAACT | | M2- | 520 | TCTCAA | 712 | GTGAGGAT |
| | 136 | | CATA | | CCG | | 328 | | GCCG | | AA |
| Group | M2- | 137 | GTACAGC | 329 | GAAGTCC | Group | M2- | 521 | GCTCTAT | 713 | GCACTTCAT |
| 35 | 137 | | GGA | | TTG | 83 | 329 | | CCG | | G |
| | M2- | 138 | CGCATAG | 330 | TCCAATGC | | M2- | 522 | TGAGCTA | 714 | CTGGACAG |
| | 138 | | CAC | | CA | | 330 | | GTA | | GT |
| | M2- | 139 | ACTGGTT | 331 | ATGTCGA | | M2- | 523 | CTGTAGG | 715 | TGTTGGTCC |
| | 139 | | ATG | | GAC | | 331 | | AGC | | A |
| | M2- | 140 | TAGTCCA | 332 | CGTCGATA | | M2- | 524 | AACAGCC | 716 | AACACAGT |
| | 140 | | TCT | | GT | | 332 | | TAT | | AC |
| Group | M2- | 141 | TGACGG | 333 | GATTACA | Group | M2- | 525 | GCGCATC | 717 | TTCAAGGA |
| 36 | 141 | | TTGC | | GGT | 84 | 333 | | GTT | | GA |
| | M2- | 142 | GCTAACC | 334 | TCCGGAG | | M2- | 526 | TTAAGC | 718 | CCGTCTAG |
| | 142 | | GTG | | AAC | | 334 | | ATCC | | TG |
| | M2- | 143 | ATGGTTG | 335 | ATACCGT | | M2- | 527 | CGCGTAT | 719 | AGAGGATC |
| | 143 | | ACT | | CTA | | 335 | | CAG | | CT |
| | M2- | 144 | CACTCAA | 336 | CGGATTCT | | M2- | 528 | AATTCG | 720 | GATCTCCT |
| | 144 | | CAA | | CG | | 336 | | GAGA | | AC |
| Group | M2- | 145 | TAAGCTA | 337 | AACTGAC | Group | M2- | 529 | AGCGAA | 721 | ACCATGAG |
| 37 | 145 | | CCA | | CGT | 85 | 337 | | TGAT | | AC |
| | M2- | 146 | AGCTGAG | 338 | GTACTTAT | | M2- | 530 | CATCGT | 722 | TGTTCATC |
| | 146 | | GTG | | CC | | 338 | | CCTG | | TG |
| | M2- | 147 | CTTCTCC | 339 | CGTACGTA | | M2- | 531 | GCGTTCA | 723 | CTAGACCT |
| | 147 | | AGC | | AG | | 339 | | AGA | | GT |
| | M2- | 148 | GCGAAGT | 340 | TCGGACG | | M2- | 532 | TTAACGG | 724 | GAGCGTGA |
| | 148 | | TAT | | GTA | | 340 | | TCC | | CA |
| Group | M2- | 149 | ATCCATG | 341 | AGGACAG | Group | M2- | 533 | CGGATAT | 725 | TGCTGGAT |
| 38 | 149 | | TCC | | AGG | 86 | 341 | | CAA | | CT |
| | M2- | 150 | TAGGTCT | 342 | TCTGACA | | M2- | 534 | GTACCG | 726 | AAGGCACA |
| | 150 | | CGG | | CAA | | 342 | | AAGC | | AC |
| | M2- | 151 | GCATGAA | 343 | GAATGTT | | M2- | 535 | TACTACC | 727 | CTAATCTGG |
| | 151 | | GAA | | GTC | | 343 | | GTT | | A |
| | M2- | 152 | CGTACGC | 344 | CTCCTGC | | M2- | 536 | ACTGGTG | 728 | GCTCATGCT |
| | 152 | | ATT | | TCT | | 344 | | TCG | | G |
| Group | M2- | 153 | GTGAATT | 345 | AGAAGAT | Group | M2- | 537 | CGTAATT | 729 | GATCCTCAA |
| 39 | 153 | | GAC | | GCA | 87 | 345 | | CAG | | C |
| | M2- | 154 | TACCTAG | 346 | GTTCCGCT | | M2- | 538 | GAAGCG | 730 | TGCAGCAG |
| | 154 | | ACT | | TC | | 346 | | GTCA | | CA |
| | M2- | 155 | AGATGCC | 347 | CACTATGA | | M2- | 539 | TTCCGAA | 731 | CTAGTGTTG |
| | 155 | | TGG | | GT | | 347 | | GGT | | G |
| | M2- | 156 | CCTGCGA | 348 | TCGGTCA | | M2- | 540 | ACGTTCC | 732 | ACGTAAGC |
| | 156 | | CTA | | CAG | | 348 | | ATC | | TT |
| Group | M2- | 157 | CCGGTCA | 349 | GTCAGGC | Group | M2- | 541 | TAGGAC | 733 | AGTGGATC |
| 40 | 157 | | ACT | | ATG | | 349 | | CGTC | | AA |
| | M2- | 158 | GTAAGAT | 350 | TCAGTCA | 88 | M2- | 542 | AGTCCT | 734 | TACATCAA |
| | 158 | | GAG | | TGT | | 350 | | GTAA | | CG |
| | M2- | 159 | TACTCGC | 351 | AAGCAAT | | M2- | 543 | CTAAGG | 735 | CTACCGCG |
| | 159 | | CGA | | GAC | | 351 | | AAGG | | TT |
| | M2- | 160 | AGTCATG | 352 | CGTTCTG | | M2- | 544 | GCCTTAT | 736 | GCGTATGT |
| | 160 | | TTC | | CCA | | 352 | | CCT | | GC |
| Group | M2- | 161 | TCGAGGT | 353 | GAAGACG | Group | M2- | 545 | CTTGGTG | 737 | ACACTAGC |
| 41 | 161 | | TCC | | AGT | 89 | 353 | | ACC | | CT |
| | M2- | 162 | GATGCTA | 354 | CTTCTTC | | M2- | 546 | AGAACA | 738 | TGTGAGCG |
| | 162 | | GAA | | CTC | | 354 | | CTTG | | AA |
| | M2- | 163 | ATCTAAC | 355 | AGGAGAA | | M2- | 547 | GCGCACA | 739 | CACTCCATG |
| | 163 | | CTG | | GAA | | 355 | | CAA | | C |
| | M2- | 164 | CGACTC | 356 | TCCTCGT | | M2- | 548 | TACTTGT | 740 | GTGAGTTA |
| | 164 | | GAGT | | TCG | | 356 | | GGT | | TG |
| Group | M2- | 165 | ACCATAC | 357 | ACATGCG | Group | M2- | 549 | TTCAACA | 741 | TACTCGGA |
| 42 | 165 | | TCC | | TAA | 90 | 357 | | AGC | | AG |
| | M2- | 166 | CGGTGTA | 358 | TGCAAGT | | M2- | 550 | CATGCAC | 742 | ATTGGCCTG |
| | 166 | | CAA | | AGC | | 358 | | GCT | | C |
| | M2- | 167 | GTTGCG | 359 | CTTCCAA | | M2- | 551 | GCATGTG | 743 | CGGCATTGT |
| | 167 | | GAGT | | GCT | | 359 | | TTA | | T |
| | M2- | 168 | TAACACT | 360 | GAGGTTC | | M2- | 552 | AGGCTGT | 744 | GCAATAAC |
| | 168 | | GTG | | CTG | | 360 | | CAG | | CA |
| Group | M2- | 169 | CAAGTAA | 361 | CACGTTG | Group | M2- | 553 | TTGTAC | 745 | GTAGCGAG |
| 43 | 169 | | CGG | | CAG | 91 | 361 | | GGCT | | TT |
| | M2- | 170 | TCGAGT | 362 | GTGAACA | | M2- | 554 | CGAGTT | 746 | AACCGTTC |
| | 170 | | GTAT | | AGA | | 362 | | AAGG | | GA |
| | M2- | 171 | GTTCCGT | 363 | TGTTGGT | | M2- | 555 | GATCCAC | 747 | TCTATCGTA |
| | 171 | | ACC | | GTC | | 363 | | TTA | | C |
| | M2- | 172 | AGCTACC | 364 | ACACCAC | | M2- | 556 | ACCAGGT | 748 | CGGTAACA |
| | 172 | | GTA | | TCT | | 364 | | CAC | | CG |
| Group | M2- | 173 | TGATACG | 365 | AGACTCG | Group | M2- | 557 | TCCACG | 749 | GCAACTTG |
| 44 | 173 | | CGG | | CGA | 92 | 365 | | GTTC | | TC |
| | M2- | 174 | CCGACA | 366 | TAGTGAA | | M2- | 558 | ATATTCT | 750 | ATGGAGAC |
| | 174 | | CATC | | TCG | | 366 | | CGG | | GG |
| | M2- | 175 | GTTGGTT | 367 | CCTGCTT | | M2- | 559 | CGGCGA | 751 | CGTCTAGT |
| | 175 | | GAA | | AAT | | 367 | | AGAT | | AA |
| | M2- | 176 | AACCTGA | 368 | GTCAAGC | | M2- | 560 | GATGATC | 752 | TACTGCCA |
| | 176 | | TCT | | GTC | | 368 | | ACA | | CT |
| Group | M2- | 177 | ATTGAGC | 369 | CCTATAAT | Group | M2- | 561 | GACCTAC | 753 | GATCAACA |
| 45 | 177 | | ACT | | GC | 93 | 369 | | TAT | | AG |
| | M2- | 178 | TGAATTG | 370 | AGCTACC | | M2- | 562 | TGGAATA | 754 | ACATTCTTC |
| | 178 | | TGC | | AAT | | 370 | | CCA | | C |
| | M2- | 179 | GACCGAT | 371 | TTACGTG | | M2- | 563 | ATTGCCG | 755 | TTGGCTGC |
| | 179 | | GTG | | CCG | | 371 | | AGC | | GT |
| | M2- | 180 | CCGTCCA | 372 | GAGGCGT | | M2- | 564 | CCATGGT | 756 | CGCAGGAG |
| | 180 | | CAA | | GTA | | 372 | | GTG | | TA |
| Group | M2- | 181 | TAAGACG | 373 | GTTGCCAT | Group | M2- | 565 | AGATTGT | 757 | CGTGTATGT |
| 46 | 181 | | CCA | | TA | 94 | 373 | | GCG | | C |
| | M2- | 182 | CTCTCTC | 374 | ACCAATT | | M2- | 566 | GATGAAG | 758 | GTCTGTCA |
| | 182 | | TAG | | CGG | | 374 | | TGC | | AG |
| | M2- | 183 | GCTAGAT | 375 | CGATGAG | | M2- | 567 | TCGACTA | 759 | TCAACCGC |
| | 183 | | AGT | | ACT | | 375 | | CAT | | CA |
| | M2- | 184 | AGGCTG | 376 | TAGCTGC | | M2- | 568 | CTCCGCC | 760 | AAGCAGAT |
| | 184 | | AGTC | | GAC | | 376 | | ATA | | GT |
| Group | M2- | 185 | TCTAACG | 377 | CGAGTTG | Group | M2- | 569 | AGTTGCC | 761 | GACAAGTG |
| 47 | 185 | | TGT | | AAT | 95 | 377 | | GGA | | GT |
| | M2- | 186 | AGGCCT | 378 | GACACCT | | M2- | 570 | CCAACTT | 762 | CTGGTACA |
| | 186 | | CCAA | | TCC | | 378 | | AAC | | CA |
| | M2- | 187 | GAATTGA | 379 | TTGCAAC | | M2- | 571 | TAGGTGA | 763 | TGACCTGCT |
| | 187 | | ACC | | GTA | | 379 | | TCT | | G |
| | M2- | 188 | CTCGGAT | 380 | ACTTGGA | | M2- | 572 | GTCCAAG | 764 | ACTTGCATA |
| | 188 | | GTG | | CGG | | 380 | | CTG | | C |
| Group | M2- | 189 | GTTCTAA | 381 | GTCATCG | Group | M2- | 573 | TCCAAGC | 765 | AGTGAGGT |
| 48 | 189 | | CTC | | CGT | 96 | 381 | | TCC | | CA |
| | M2- | 190 | CCAACTC | 382 | TAGGAAT | | M2- | 574 | AGGTTCG | 766 | CTACTAAGG |
| | 190 | | GCT | | GCA | | 382 | | GTG | | C |
| | M2- | 191 | AGGTAGT | 383 | CCTTGTC | | M2- | 575 | CTAGGTA | 767 | GAGTCCTAT |
| | 191 | | AAG | | TAC | | 383 | | AGA | | T |
| | M2- | 192 | TACGGC | 384 | AGACCGA | | M2- | 576 | GATCCAT | 768 | TCCAGTCC |
| | 192 | | GTGA | | ATG | | 384 | | CAT | | AG |

Note:
1. Bold fonts in two columns of “index corresponding to phosphorylation end” and “index corresponding to non-phosphorylation end″ in the table are indexes that are marked as excellent indexes; normal fonts are indexes that are marked as normal indexes; and the underlined parts are indexes that are marked as unqualified indexes.
2. Bold fonts in the column of ″balanced combination” in the table are 4-base balanced combinations that are marked as excellent groups; normal fonts are 4-base balanced combinations that are marked as normal groups; and the underlined parts are 4-base balanced combinations that are marked as unqualified groups.

In order to implement the above objective, a second aspect of the present disclosure provides a kit for constructing a DNA library. The above kit includes an amplification primer composition, where the above amplification primer composition includes a combination of a plurality of amplification primer pairs and a bubble adapter in the method for constructing a DNA library based on an MGI platform.

In order to implement the above objective, a third aspect of the present disclosure provides a sequencing library. The above sequencing library is a library that is constructed utilizing the above method for constructing a DNA library based on an MGI platform.

Utilizing the technical solutions of the present disclosure, dual indexes showing stable library output and whole-genome library sequencing data are further screened out from 96 groups of 4-base balanced fixed dual indexes (shown in Table 1 in the present disclosure) and another 96 groups of 4-base balanced fixed dual indexes (shown in Table 2 in the present disclosure) provided in CN111910258B.

The dual indexes of which normalized values for library output and whole-genome library sequencing data are 0.85-1.15 are marked as the excellent indexes; the dual indexes of which normalized values for library output and/or whole-genome library sequencing data are >1.2 or <0.8 are marked as the unqualified indexes; and other indexes are marked as the normal indexes. Since one 4-base balanced group totally includes 4 pairs of the dual indexes, the 4-base balanced group totally obtains 4 marks.

Then, the 4-base balanced group is further tagged, according to marking situations of the 4-base balanced dual indexes. If the 4 marks in the 4-base balanced group are all excellent indexes, the 4-base balanced group is marked as an excellent group. If the 4 marks in the 4-base balanced group have excellent indexes and normal indexes or only have the normal indexes, the 4-base balanced group is marked as a normal group. If the 4 marks in the 4-base balanced group have unqualified indexes, the 4-base balanced group is marked as an unqualified group. According to the above screening standard, in the present disclosure, 58 excellent groups, 51 normal groups, and 83 unqualified groups are screened out from the 192 groups of 4-base balanced fixed dual indexes in Table 1 and Table 2. In order to improve the quality of sequencing data, when the above indexes are used, the excellent groups are the first selected, the normal groups are the second selected, and the unqualified groups are eliminated.

The development and application of the present disclosure provide more dual index adapters for an MGI platform, expand the application range and field of an MGI sequencing platform, and solve the problem of easy occurrence of sample crosstalk when the MGI platform deals with the loading of mass sequencing samples (e.g., 384 samples), thereby improving the sequencing quality of the MGI platform.

### BRIEF DESCRIPTION OF DRAWINGS

The drawings, which form a part of the present disclosure, are used to provide a further understanding of the present disclosure. The exemplary embodiments of the present disclosure and the description thereof are used to explain the present disclosure, but do not constitute improper limitations to the present disclosure. In the drawings:

FIG. 1 shows assessment of library output and whole-genome library sequencing data splitting of 4-base balanced combinations (shown in Table 1 in the present disclosure) of 96 groups of 4-base balanced fixed unique dual indexes in Patent CN111910258B.

FIG. 2 shows a ratio of excellent groups, normal groups, and unqualified groups in 96 groups of 4-base balanced combinations (shown in Table 1 in the present disclosure) in Patent CN111910258B.

FIG. 3 shows assessment of library output and whole-genome library sequencing data splitting of 4-base balanced combinations (shown in Table 2 in the present disclosure) of another set of 96 groups of fixed unique dual indexes in the present disclosure.

FIG. 4 shows a ratio of excellent groups, normal groups, and unqualified groups in another set of 96 groups of 4-base balanced combinations (shown in Table 2 in the present disclosure) in the present disclosure.

### DESCRIPTION OF EMBODIMENTS

It is to be noted that the embodiments in the disclosure and the features in the embodiments may be combined with one another without conflict. The present disclosure will be described below in detail with reference to the embodiments.

### Term Explanation

4-base balanced group: in this application, a group consists of four dual indexes. This group includes a total of four upstream indexes and four downstream indexes. Both the upstream and downstream indexes are composed of 10 bases. For the first to the tenth positions in the four upstream indexes each of the bases A, T, C, and G appears exactly once. Similarly, for the first and tenth positions in the four downstream indexes, each of the bases A, T, C, and G also appears exactly once.

Fixed unique dual indexes in 4-base balanced groups: in this application, the dual indexes in the 4-base balanced group have fixed upstream and downstream indexes pairings that cannot be interchangeably replaced.

Assessment for 4-base balanced group: first, evaluate each dual indexes in the four-base balanced group from the perspectives of library output and whole-genome sequencing data splitting. Mark each paired dual indexes as excellent indexes, normal indexes, or unqualified indexes. Then, based on the marks of the four dual indexes within the four-base balanced group, evaluate the overall groups and mark it as an excellent group, normal group, or unqualified group.

Library output normalization: a sample is broken through ultrasound to obtain 200-400 bp DNA fragments; different index adapters are used to construct different DNA libraries for equivalent amount of DNA, all conditions are the same except that the indexes are different; and a numerical value obtained by dividing the library output of a single DNA library by an average of all DNA libraries is a normalized value for the libraries, and the closer the value is to 1, the better.

Whole-genome sequencing data splitting normalization: library construction is performed on the same sample utilizing different indexes, obtaining DNA libraries of different indexes; and the above construction process has the same conditions except that the indexes are different. Different libraries constructed by different indexes are mixed with equal mass and then sent for sequencing; and generally, 0.2-0.5 GB of data is arranged for each dual indexes. A numerical value obtained by dividing the data produced through sequencing of each library by an average of the data produced through sequencing of all the libraries is a whole-genome sequencing data splitting normalized value; and the closer the value is to 1, the better.

Index pairs: also known as dual indexes, include 5′ end library index and 3′ end library index, and are used to distinguish between different samples under test.

Since its launch, the MGI sequencer has, for the first time, surpassed the installation volume of Illumina sequencers in terms of new installations in the domestic market. Although MGI has won the favor of the domestic market by virtue of two factors: domestic production and price, the platform mainly uses sequencing adapters converted by App-A for loading. From the perspective of client applications, Nanodigmbio optimizes a solution of balancing unique dual indexes in groups of 4 of MGI, shown in Patent CN111910258B.

As the MGISEQ Dual Indexes solution for MGI, developed utilizing Patent CN111910258B, is used and tested in the market, it has been found that good sequencing quality can be achieved when an index sequence meets the minimal base balance required by MGI. Specifically, this requires that the proportion of any single base be at least 12.5%.

Since MGI loading is more stringent in terms of base balance than Illumina loading, the problem mainly solved in CN111910258B is that 4-balanced or 8-balanced index adapter combinations facilitate MGI loading.

As the products developed by the Patent CN111910258B are used, the inventor finds that although the products developed by the CN111910258B solve the problem of base balance on the MGI sequencing platform, the library construction efficiency and the loading data splitting of equal-ratio mixing of some fixed dual index combination primers fluctuate greatly, seriously affecting the sequencing quality of the MGI platform, thus not meeting client requirements. The

CN111910258B solves the problem of absolute balance of bases in groups of 4 or 8 and the problem of possibly affecting library output when different indexes pass through a secondary structure, dimensional fluctuations of library output through assessment and practical application are relatively stable. However, due to a large difference in base reads by a sequencer itself, the data from specific index combinations of a fixation group fluctuates greatly in data splitting after equal-ratio mixing.

Since the MGI sequencer requires that the proportion of a base cannot be less than 12.5%, when a large number of samples (e.g., 384 samples) are loaded at the same, data output of index primer combinations of some samples is not ideal, thus affecting client satisfaction. Furthermore, in order to meet the simultaneously loading of a large number of samples, before the base read preference is not solved by MGI currently, sequencers that DNBSEQ-T7 outputs 6Tb data and DNBSEQ-T20X2 outputs 72Tb data are launched successively based on the current throughput of sequencers. On the basis of the Patent CN111910258B, in the present disclosure, in order to meet clients for better product satisfaction, on the basis of 4-base balanced fixed dual indexes in groups of 4, strict screening and evaluation are performed again on two dimensions of library output and sequencing data splitting, and better use suggestions are given.

As mentioned in the Background, dual sequencing indexes of the MGI platform in the prior art has the problem of unsatisfactory data output when dealing with the simultaneous loading of a large number of samples (e.g., 384 samples). In the present disclosure, on the basis of 4-base balanced fixed dual indexes in groups of 4, strict evaluation and screening are performed on the two dimensions of library output and whole-genome library sequencing data splitting of the DNA library constructed by different indexes. The method of the present disclosure provides a new solution for the selection of sequencing indexes of the MGI platform, and improves the accuracy of the sequencing data when a large number of samples (e.g., 384 samples) are loaded at the same time, thereby promoting the development of MGI platform sequencing. Based on the above problems, the inventor proposes a series of protective solutions of the present disclosure. A first typical implementation of the present disclosure provides a method for constructing a DNA library based on an MGI platform. The construction method includes: a bubble adapter is ligated to a target sample to obtain a ligation product.

Library construction is performed on the ligation product utilizing an amplification primer pair, obtaining an MGI platform based amplified library with dual indexes.

The amplification primer pair has the dual indexes and includes a 5′ end library index and a 3′ end library index.

The 5′ end library index is selected from indexes corresponding to any one of groups of 4-base-balanced phosphorylation ends in Table 1 or Table 2, and the above 3′ end library index is selected from indexes corresponding to fixedly-matched non-phosphorylation ends corresponding to the above 5′ end library index group in Table 1 or Table 2.

The above 4-base-balance refers to balance of index sequences in groups of 4, at each position from the first to the tenth in the index sequence, there is one base of A, one base of T, one base of C, and one base of G.

The above bubble adapter comprises a first adapter sequence and a second adapter sequence, the above first adapter sequence is SEQ ID NO: 769, and the above second adapter sequence is SEQ ID NO: 770.

Each of the above amplification primer pairs further includes a 5′ end universal amplification sequence and a 3′ end universal amplification sequence, the above 5′ end universal amplification sequence includes a universal sequence located upstream of the above 5′ end library index and a universal sequence located downstream of the above 5′ end library index, and the above 3′ end universal amplification sequence comprises a universal sequence located upstream of the above 3′ end library index and a universal sequence located downstream of the above 3′ end library index. The universal sequence located upstream of the above 5′ end library index is SEQ ID NO: 773, the universal sequence located downstream of the above 5′ end library index is SEQ ID NO: 774; the universal sequence located upstream of the above 3′ end library index is SEQ ID NO: 775, and the universal sequence located downstream of the above 3′ end library index is SEQ ID NO: 771, and is used in combination with the first adapter sequence shown in SEQ ID NO: 769 and the second adapter sequence shown in SEQ ID NO: 770.

A second typical implementation of the present disclosure provides a kit for constructing a DNA library. The kit includes an amplification primer composition, where the amplification primer composition includes a combination of a plurality of amplification primer pairs and a bubble adapter in the method for constructing a DNA library based on an MGI platform.

A third typical implementation of the present disclosure provides a sequencing library. The sequencing library is a library that is constructed according to the method for constructing a DNA library based on an MGI platform.

The beneficial effects of the present disclosure are further described in detail below with reference to specific embodiments.

### Embodiment 1 Assessment of Library Output and Whole-Genome Library Sequencing Data Splitting of Unique Dual Index Library Construction Adapters of Different Combinations in Table 1 of CN111910258B

Specific experiment steps were shown as follows.

### Step I: Sample Fragmentation

A Covaris™ series DNA ultrasonic disruptor was used to fragment genomic DNA standard samples (Promega, G1521) to an average fragment size of 250-300 bp.

Step II: End repair & A tailing (NadPrep® DNA universal library construction kit, article number: #1002101, Nanodigmbio). Specific operation steps were shown as follows.
1. End Repair & A-Tailing Buffer was taken and melted at normal temperature, well mixed, and placed on ice for later use.
2. End Repair & A-Tailing Enzyme was taken and placed on ice for natural melting, well mixed, and subjected to instantaneous centrifugation for later use.
3. According to the following table, a reaction system was prepared in a 0.2 mL PCR tube placed on ice, and the reaction system was shown in Table 3.

TABLE 3

| | |
|---|---|
| Fragmented DNA | 40 μL(10 ng) |
| End Repair & A-Tailing Buffer | 6 μL |
| End Repair & A-Tailing Enzyme | 4 μL |
| Total volume | 50 μL |

4. Well mixing was performed, and instantaneous centrifugation was performed to cause all reaction liquid to be placed at the bottom of the PCR tube.
5. The following reaction procedures were started on a PCR instrument, a reaction tube was placed in the PCR instrument when a temperature was stabilized to 20° C., and the reaction procedures were shown in Table 4.

TABLE 4

| | |
|---|---|
| 20° C. | 30 min |
| 65° C. | 30 min |
| 10° C. | Hold |

### Step III: Adapter Ligation

1. NadPrep® Universal Stubby Adapter was formed through annealing of primers SEQ ID NO: 770 and SEQ ID NO: 769; after the two primers were mixed with equal molar, high-temperature incubation was performed for 2 min at 95° C., and then the temperature was slowly cooled to 20° C., so as to form the NadPrep® Universal Stubby Adapter. A sequence of SEQ ID NO: 770 was ttgtcttcctaacaggaacgacatggctacgatccgact*t; and * represented thio modification.

A sequence of SEQ ID NO: 769 was/5Phos/agtcggaggccaagcggtcttaggaagacaa, and/5Phos/represented phosphorylation modification; and the two sequences were the same in CN 111910258 B, and had the same functions and effects.
2. Ligation Buffer was taken and melted at normal temperature, well mixed, and placed on ice for later use.
3. DNA Ligase was taken and placed on ice for natural melting, well mixed, and subjected to instantaneous centrifugation for later use.
4. The PCR reaction tube in step II was taken out from the PCR instrument and placed on ice, a reaction system was prepared according to the following table, and the reaction system was shown in Table 5.

TABLE 5

| | |
|---|---|
| Reaction product in step II | 50 μL |
| NadPrep ® Universal Stubby Adapter | 2 μL |
| Ligation Buffer | 26 μL |
| DNA Ligase | 2 μL |
| Total volume | 80 μL |

4. Well mixing was performed, and instantaneous centrifugation was performed to cause all reaction liquid to be placed at the bottom of the PCR tube.
5. The following reaction procedures were started on a PCR instrument, a reaction tube was placed in the PCR instrument when a temperature was stabilized to 20° C., and the reaction procedures were shown in Table 6.

TABLE 6

| | |
|---|---|
| 20° C. | 15 min |
| 4° C. | Hold |

### Step IV: Purification of Ligation Product

1. NadPrep® SP Beads are taken out in advance for vortex mixing, and used after being balanced for 30 min at room temperature.
2. 40 μL of the NadPrep® SP Beads was added to the ligation reaction product in step III, well mixed, and incubated for 5-10 min at 25° C.
3. The PCR tube was subjected to instantaneous centrifugation and then placed on a magnetic frame for 5 min until the liquid was completely clear, and supernatant was pipetted utilizing a pipette and then discarded.
4. 150 μL of 80% ethanol was slowly added along a sidewall of the PCR tube, taking care not to disturb magnetic beads, and the PCR tube was allowed to stand for 30 sec; and supernatant was pipetted utilizing a pipette and then discarded.
5. S4 was repeated once.
6. The PCR tube was subjected to instantaneous centrifugation and then placed on the magnetic frame, a 10 μL pipette tip was used to remove a small amount of residual ethanol, taking care not to pipet the magnetic beads.
7. A cap of the PCR tube was opened, and the tube was allowed to stand at room temperature for about 5 min until the ethanol was completely volatilized.
8. The PCR tube was removed out, 20 μL of Nuclease Free Water was added to the PCR tube, and the tube entered step V with the magnetic beads.

### Step V: Amplification of NadPrep® Universal Stubby Adapter Ligation Product

2× HiFi PCR Master Mix and NadPrep® Universal Stubby Adapter Primer Mix were taken out and placed on ice for natural melting, well mixed, and subjected to instantaneous centrifugation for later use. The NadPrep® Universal Stubby Adapter Primer Mix was formed by mixing primers SEQ ID NO: 771 and SEQ ID NO: 772 with equal molar.

A sequence of SEQ ID NO: 771 was
TTGTCTTCCTAAGACCGCTTGGCC.
A sequence of SEQ ID NO: 772 was
CACAGAACGACATGGCTACGA.

2. According to the following table, a reaction system was prepared in a 0.2 mL PCR tube placed on ice (sequentially adding from top to bottom), and the reaction system was shown in Table 7.

TABLE 7

| | |
|---|---|
| Ligation product purified in step IV | 20 μL |
| NadPrep ® Universal Stubby Adapter Primer Mix | 5 μL |
| 2X HiFi PCR Master Mix | 25 μL |
| Total volume | 50 μL |

3. The PCR tube was placed in the PCR instrument to start the following procedures, and the reaction procedures were shown in Table 8.

TABLE 8

| | | |
|---|---|---|
| 98° C. | 2 min | |
| 98° C. | 15 s | 5 cycles |
| 60° C. | 30 s | |
| 72° C. | 30 s | |
| 72° C. | 2 min | |
| 4° C. | Hold | |

### Step VI: Purification and Quantification of NadPrep® Universal Stubby Adapter Amplification Product

1. NadPrep® SP Beads are taken out in advance for vortex mixing, and used after being balanced for 30 min at room temperature.
2. 50 μL of NadPrep® SP Beads was added to the amplification product in step V, well mixed, and incubated for 5-10 min at 25° C.
3. The PCR tube was subjected to instantaneous centrifugation and then placed on a magnetic frame for 5 min until the liquid was completely clear, and supernatant was pipetted utilizing a pipette and then discarded.
4. 150 μL of 80% ethanol was slowly added along a sidewall of the PCR tube, taking care not to disturb magnetic beads, and the PCR tube was allowed to stand for 30 sec; and supernatant was pipetted utilizing a pipette and then discarded.
5. S4 was repeated once.
6. The PCR tube was subjected to instantaneous centrifugation and then placed on the magnetic frame, a 10 μL pipette tip was used to remove a small amount of residual ethanol, taking care not to pipet the magnetic beads.
7. A cap of the PCR tube was opened, and the tube was allowed to stand at room temperature for about 5 min until the ethanol was completely volatilized.
8. The PCR tube was removed out, 50 μL of Nuclease Free Water was added to the PCR tube, a pipette was used to suspend the magnetic beads evenly, and incubation was performed for 2 min at 25° C.
9. The PCR tube was subjected to instantaneous centrifugation and then placed on the magnetic frame for 2 min until the liquid was completely clear, and supernatant was transferred to a new 0.2 mL PCR tube utilizing the pipette, taking care not to pipet the magnetic beads.
10. The purified product was quantified utilizing methods such as Qubit or quantitative PCR, etc.
11. The Nuclease Free Water was used to dilute the purified product, and a final concentration was 1 ng/μL.

### Step VII: Amplification of NadPrep® Universal MDI-Index Primer Mix

1. 2× HiFi PCR Master Mix and NadPrep® Universal MDI-Index Primer Mix were taken out and placed on ice for natural melting, well mixed, and subjected to instantaneous centrifugation for later use. The NadPrep® Universal MDI-Index Primer Mix was formed by mixing a Primer 1 and a Primer 2 with equal molar.

Sequences of the Primer 1 included 5′-SEQ ID NO: 773-nnnnnnnnnn-SEQ ID NO: 774-3′, with a 5′ end having phosphorylation modification; a sequence of SEQ ID NO: 773 was ctctcagtacgtcagcagtt; a sequence of SEQ ID NO: 774 was caactccttggctcacagaacgacatggctacga; and nnnnnnnnnn was selected from SEQ ID NO: 776-1159 (forward application, shown in Table 1 of the present disclosure).

Sequences of the Primer 2 included 5′-SEQ ID NO: 775-nnnnnnnnn-SEQ ID NO: 771-3′; a sequence of SEQ ID NO: 775 was gcatggcgaccttatcag; a sequence of SEQ ID NO: 771 was ttgtcttcctaagaccgcttggcc; and nnnnnnnnnn was selected from SEQ ID NO: 1159-776 (forward application, shown in Table 1 of the present disclosure).

It was to be noted that, the nnnnnnnnnn of the Primer 1 and the nnnnnnnnnn of the Primer 2 had a one-to-one correspondence fixedly-matched relationship. For example, when the nnnnnnnnnn of the Primer 1 was the sequence shown in SEQ ID NO: 1 in Table 1, the nnnnnnnnnn of the Primer 2 was the sequence shown in SEQ ID NO: 384 in Table 1.

For another example, when the nnnnnnnnnn of the Primer 1 was the sequence shown in SEQ ID NO: 2 in Table 1, the nnnnnnnnnn of the Primer 2 was the sequence shown in SEQ ID NO: 383 in Table 1.

For yet another example, when the nnnnnnnnnn of the Primer 1 was the sequence shown in SEQ ID NO: 3 in Table 1, the nnnnnnnnnn of the Primer 2 was the sequence shown in SEQ ID NO: 382 in Table 1, and so on; and 384 pairs of fixedly-matched dual index combinations were totally obtained and used for library construction. Specific correspondence relationships were shown in

Table 1. Therefore, 4-base balance was ensured, and specific combinations of upstream and downstream indexes were limited. This was done to assess the two indicators of library output and sequencing data splitting through such determined relationships.

It was to be noted that, the index sequences in Table 1 of the present disclosure were derived from Table 1 in the Patent CN 111910258 B; and the indexes in Table 1 in the Patent CN 111910258 B were fixedly matched to obtain 384 fixedly-matched upstream and downstream index pairs. In this embodiment, the balance of library output and whole-genome library sequencing data was determined further based on the 384 fixedly-matched upstream and downstream index pairs in Table 1 of the present disclosure, so as to assess whether the 384 fixedly-matched upstream and downstream index pairs were excellent, normal, or unqualified.
2. According to the following table, a reaction system was prepared in a 0.2 mL PCR tube placed on ice (sequentially adding from top to bottom), and the reaction system was shown in Table 9.

TABLE 9

| | |
|---|---|
| Ligation product purified in step VI | 10 μL |
| NadPrep ® Universal MDI-Index Primer Mix | 2.5 μL |
| 2X HiFi PCR Master Mix | 12.5 μL |
| Total volume | 25 μL |

3. The PCR tube was placed in the PCR instrument to start the following procedures, and the reaction procedures were shown in Table 10.

TABLE 10

| | | |
|---|---|---|
| 98° C. | 2 min | |
| 98° C. | 15 s | 8 cycles |
| 60° C. | 30 s | |
| 72° C. | 30 s | |
| 72° C. | 2 min | |
| 4° C. | Hold | |

### Step VIII: Purification and Quantification of Amplified Library

1. NadPrep® SP Beads are taken out in advance for vortex mixing, and used after being balanced for 30 min at room temperature.
2. 25 μL of NadPrep® SP Beads was added to the amplification product in step VII, well mixed, and incubated for 5-10 min at 25° C.
3. The PCR tube was subjected to instantaneous centrifugation and then placed on a magnetic frame for 5 min until the liquid was completely clear, and supernatant was pipetted utilizing a pipette and then discarded.
4. 150 μL of 80% ethanol was slowly added along a sidewall of the PCR tube, taking care not to disturb magnetic beads, and the PCR tube was allowed to stand for 30 sec; and supernatant was pipetted utilizing a pipette and then discarded.
5. S4 was repeated once.
6. The PCR tube was subjected to instantaneous centrifugation and then placed on the magnetic frame, a 10 μL pipette tip was used to remove a small amount of residual ethanol, taking care not to pipet the magnetic beads.
7. A cap of the PCR tube was opened, and the tube was allowed to stand at room temperature for about 5 min until the ethanol was completely volatilized.
8. The PCR tube was removed out, 30 μL of a TE Solution was added to the PCR tube, a pipette was used to suspend the magnetic beads evenly, and incubation was performed for 2 min at 25° C.
9. The PCR tube was subjected to instantaneous centrifugation and then placed on the magnetic frame for 2 min until the liquid was completely clear, and supernatant was transferred to a new 0.2 mL PCR tube utilizing the pipette, taking care not to pipet the magnetic beads.
10. The purified product was quantified utilizing methods such as Qubit or quantitative PCR, etc.
   Step IX: Direct sequencing after equal-ratio mixing

5 ng of each library was taken and mixed together, 200 ng of the mixed libraries after equal-ratio mixing was taken to arrange loading sequencing, each library was arranged for 0.5 GB for loading, and loading sequencing was performed on an MGI2000 sequencer.

### S10: Whole-Genome Library Sequencing Data Splitting

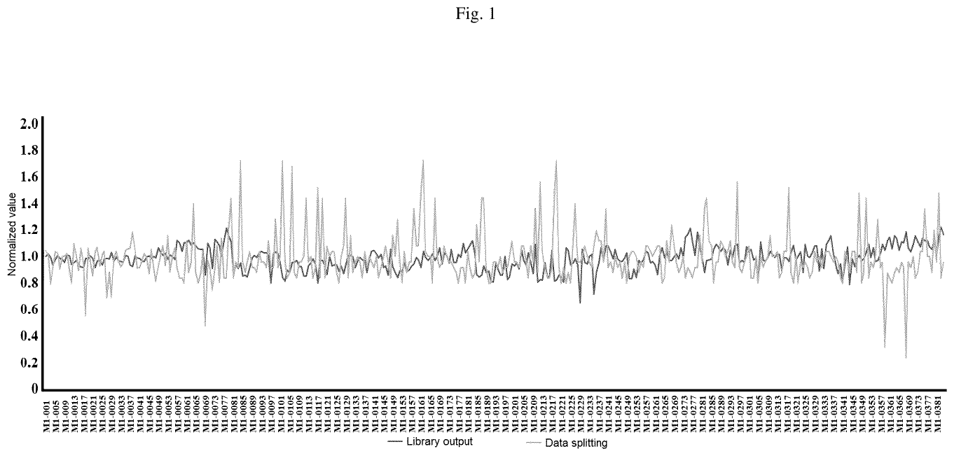

Data splitting was performed according to the 384 pairs of dual indexes Step VII, and library output and whole-genome library sequencing data splitting were assessed; and normalization processing was performed on the library output and whole-genome library sequencing data corresponding to each pair of the indexes, obtaining assessed data corresponding to each pair of the indexes, as shown in FIG. 1. In the present disclosure, classification and statistics were performed on the dual indexes (index pairs) in Table 1 according to classification standards of “0.85≤normalized value ≤1.15”, “0.80≤normalized value <0.85 or 1.15<normalized value ≤1.20”, and “normalized value >1.2 or normalized value <0.80” from two perspectives of library output and data splitting. Statistical results were shown in Table 11.

TABLE 11
Data for library output and data splitting
of each dual indexes after normalization

| | Normalized | | |
|---|---|---|---|
| | value > | 0.80 ≤ normalized | 0.85 ≤ |
| | 1.2 or normalized | value < 0.85 | normalized |
| | value < 0.80 | or 1.15 < normalized | value ≤ 1.15 |
| | Unqualified | value ≤ 1.20 | Excellent |
| | indexes | Normal indexes | indexes |
| Library | 6 | 29 | 349 |
| normalization | | | |
| Data splitting | 44 | 32 | 308 |
| normalization | | | |

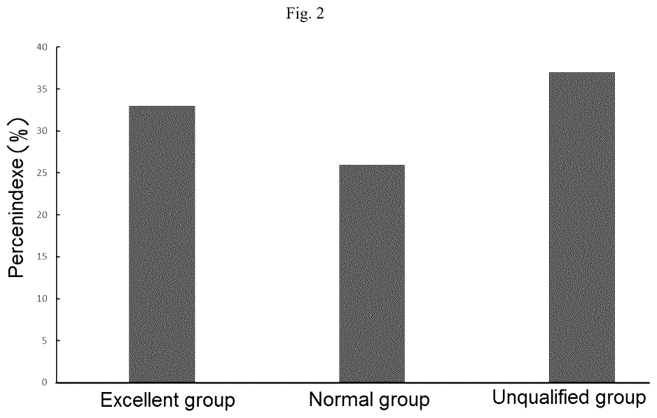

In order to optimize the use of the index combinations, in the present disclosure, according to a 4-base balance rule, the 384 pairs of fixedly-matched dual index combinations were classified into 96 4-base balanced groups (as described in S7 of Embodiment 1), and each pair of the dual indexes was further assessed from two perspectives of the uniformity of library output and the uniformity of whole-genome library sequencing data splitting. The dual indexes of which normalized values for library output and whole-genome library sequencing data were 0.85-1.15 were marked as the excellent indexes; the dual indexes of which normalized values for library output and/or whole-genome library sequencing data were >1.2 or <0.8 were marked as the unqualified indexes; and other indexes were marked as the normal indexes. Since one 4-base balanced group totally included 4 pairs of the dual indexes, the 4-base balanced group totally obtained 4 marks. Then, the 4-base balanced group was further tagged according to marking situations of the 4-base balanced dual indexes. If the 4 marks in the 4-base balanced group were all excellent indexes, the 4-base balanced group was an excellent group. If the marks in the 4-base balanced group had excellent indexes and normal indexes or only had the normal indexes, the 4-base balanced group was marked as a normal group. If the marks in the 4-base balanced group had unqualified indexes, the 4-base balanced group was an unqualified group. According to the above screening standards, in the present disclosure, 33 excellent groups (combinations marked by bold fonts in the column of balanced combination in Table 1), 26 normal groups (combinations marked by normal fonts in the column of balanced combination in Table 1), and 37 unqualified groups (combinations marked by underlines in the column of balanced combination in Table 1) were screened out from 96 groups of 4-base balanced fixed dual indexes in Table 1 (shown in Table 13). The proportion of each group with different mark was shown in FIG. 2.

It was to be noted that, the grouping of 4-base balance was performed on Table 1 of the Patent CN 111910258 B, that is, the sequences shown in SEQ ID NO: 1-4 in Table 1 of CN 111910258 B jointly formed a No. 1 4-balance group, the sequences shown in SEQ ID NO: 5-8 jointly formed a No. 2 4-balance group, the sequences shown in SEQ ID NO: 9-12 jointly formed a No. 3 4-balance group, the sequences shown in SEQ ID NO: 13-16 jointly formed a No. 4 4-balance group, and so on.

Thus, in the present disclosure, when the nnnnnnnnnn of the Primer 1 was the sequence shown in SEQ ID NO: 1-4 in Table 1, the nnnnnnnnnn of the Primer 2 was the fixedly-matched sequence shown in SEQ ID NO: 384-381 in Table 1. The sequences shown in SEQ ID NO: 1-4 also formed a 4-base balanced group, recorded as the first group of balanced combinations.

For another example, when the nnnnnnnnnn of the Primer 1 was the sequence shown in SEQ ID NO: 5-8 in Table 1, the nnnnnnnnnn of the Primer 2 was the fixedly-matched sequence shown in SEQ ID NO: 380-377 in Table 1. The sequences shown in SEQ ID NO: 5-8 also formed a 4-base balanced group, recorded as the second group of balanced combinations, and so on. The 384 pairs of fixedly-matched dual index combinations for library construction in the present disclosure were further classified into 96 4-base balanced groups. Details were shown in Table 1.

### Embodiment 2 Assessment of Balanced Library Output and Whole-Genome Library Sequencing Data Splitting of Unique Dual Index Library Construction Adapters of Different Combinations in Table 2

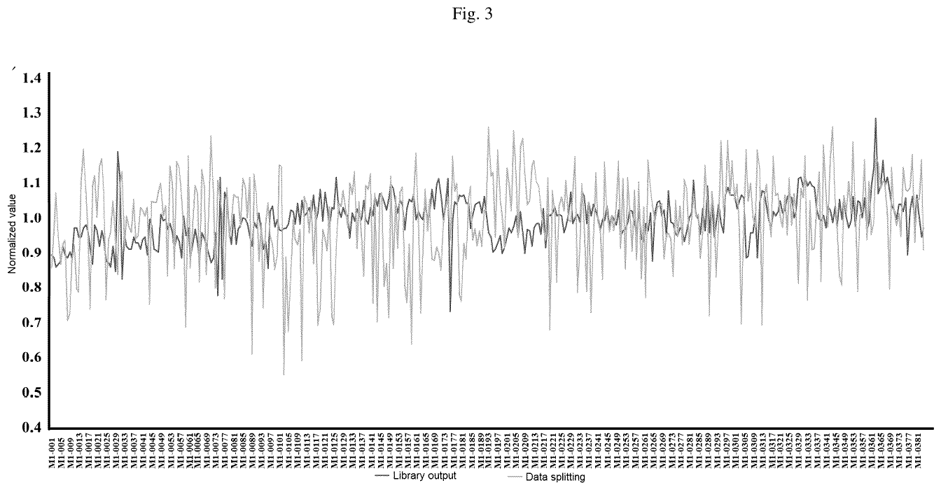

Experiment steps in this embodiment were the same as Embodiment 1. However, in sequences of Primer 1 of Embodiment 2, nnnnnnnnnn was selected from indexes corresponding to phosphorylation ends in Table 2 of the present disclosure; and in sequences of Primer 2 of Embodiment 2, nnnnnnnnnn was selected from indexes corresponding to non-phosphorylation ends in Table 2 of the present disclosure. Normalized values for library output and whole-genome library sequencing data splitting of each dual index pairs in Table 2 were shown in FIG. 3. It was to be noted that, the indexes corresponding to the phosphorylation ends in Table 2 correspondingly fixedly match the indexes corresponding to the non-phosphorylation ends on a one-to-one basis. For example, a pair of indexes numbered M2-001 was fixedly matched; and when the nnnnnnnnnn of the Primer 1 selected an index corresponding to a phosphorylation end in M2-001, the nnnnnnnnnn of the Primer 2 could only be an index corresponding to a non-phosphorylation end in M2-001.

It was to be noted that, 384 pairs of dual indexes were totally provided in Table 2. According to the 4-base balance rule, the 384 pairs of dual indexes were divided into 96 groups of 4-base balanced combinations, and the index sequences were specifically shown in Table 2.

Utilizing the screening method same as Embodiment 1, classification and statistics were performed on the dual indexes (index pairs) in Table 2 of the present disclosure from two perspectives of library output and whole-genome library sequencing data splitting. Statistical results were shown in Table 12.

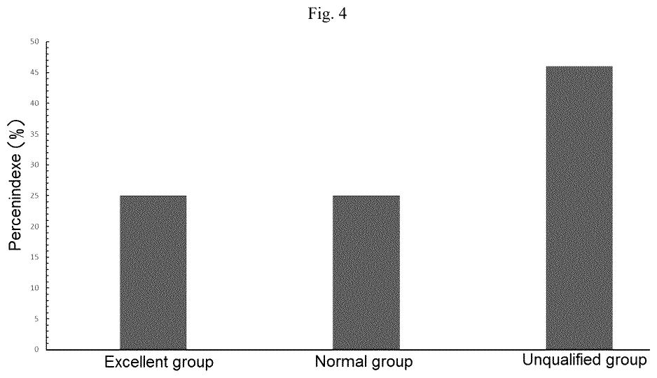

Further, the dual indexes in Table 2 of the present disclosure were respectively marked as “excellent indexes”, “normal indexes”, and “unqualified indexes” based on the marking principle same as Embodiment 1. Further, the 4-base balanced groups in Table 2 were marked according to the standard same as Embodiment 1 based on marking results. The marking results showed that, in the present disclosure, 25 excellent groups (combinations marked by bold fonts in the column of balanced combination in Table 2), 25 normal groups (combinations marked by normal fonts in the column of balanced combination in Table 2), and 46 unqualified groups (combinations marked by underlines in the column of balanced combination in Table 2) were screened out from 96 groups of 4-base balanced fixed dual indexes in Table 2 (shown in Table 13). The proportion of each group with different mark was shown in FIG. 4.

TABLE 12
Data for library output and data splitting
of each dual indexes after normalization

| | Normalized | | |
|---|---|---|---|
| | value > | 0.80 ≤ normalized | 0.85 ≤ |
| | 1.2 or normalized | value < 0.85 | normalized |
| | value < 0.80 | or 1.15 < normalized | value ≤ 1.15 |
| | Unqualified | value ≤ 1.20 | Excellent |
| | indexes | Normal indexes | indexes |
| Library | 3 | 4 | 377 |
| normalization | | | |
| Data splitting | 49 | 48 | 287 |
| normalization | | | |

It was to be noted that, the indexes in Table 2 and Table 1 were derived from different sources, and the sequences of the indexes were different, but the standards for assessing the indexes in the two tables were the same in the present disclosure. Therefore, the indexes in Table 2 and Table 1 could be subjected to loading sequencing together. By combining the assessment results of Table 1 and Table 2, 58 excellent groups, 51 normal groups, and 83 unqualified groups were totally provided in the present disclosure (shown in Table 13).

TABLE 13

| | Excellent | | | | | |
|---|---|---|---|---|---|---|
| | group | Normal group | | Unqualified group | | |
| Table | 33 | 26 | | 37 | | |
| 1 | | | | | | |
| Table | 25 | 25 | | 46 | | |
| 2 | | | | | | |
| Total | 58 | 51 | | 83 | | |
| | | | Normal | | Normal | |
| | Excellent | Excellent | index | Excellent | index | Unqualified |
| | index pairs | index pairs | pairs | index pairs | pairs | index pairs |
| Table | 132 | 66 | 38 | 85 | 12 | 51 |
| 1 | | | | | | |
| Table | 100 | 68 | 32 | 116 | 17 | 51 |
| 2 | | | | | | |
| Total | 232 | 134 | 70 | 201 | 29 | 102 |

In order to take high sequencing quality and simultaneous loading sequencing of a large number of samples into consideration, it was suggested that the present disclosure was used according to the following standard: when the number of samples did not exceed 232, four-base balanced index pairs in the excellent group were preferred. When the number of samples was 233-436, four-base balanced index pairs in the excellent group and normal group were preferred. When the number of samples was 437-768, four-base balanced index pairs in the excellent group, normal group, and unqualified group were preferred. It was to be noted that, when the number of samples was 233-567, if four-base balance was not taken into consideration, index pairs in the excellent group+excellent index pairs in the normal group+excellent index pairs in the unqualified group were preferred. Likewise, when the number of samples was 567-666, if four-base balance was not taken into consideration, excellent index pairs and normal index pairs in all groups were preferred.

Furthermore, if the number of samples was not a multiple of 4, for example, a sample size was 14, of which 12 samples used 4-base balanced dual indexes and the remaining 2 samples might not take 4-base balance into consideration.

It might be seen from the above description that, in the above embodiments of the present disclosure, the following technical effects were realized. In the present disclosure, in order to meet the increasing requirements for the types and quality of sequencing indexes in the prior art, the present disclosure provided 192 groups of 4-base balanced dual indexes, and marked the 192 4-base balanced groups as 58 excellent groups, 51 normal groups, and 83 unqualified groups from the two perspectives of library output and whole-genome data splitting. Further, the present disclosure gave recommendations for the use of the above dual indexes based on the evaluated results, and met the requirements for high sequencing quality on the basis of realizing the simultaneous loading sequencing of a large number of samples. The development and application of the present disclosure further improved the screening standard for the dual indexes of the MGI platform, such that the sequencing quality of the MGI platform was improved, and the problem of poor quality of MGI platform dual indexes when a large number of samples (e.g., 384 samples) were loaded at the same time was solved, facilitating development and improvement of an MGI sequencing platform, thereby achieving higher economic values.

The above are only the preferred embodiments of the present disclosure and are not intended to limit the present disclosure. For those skilled in the art, the present disclosure may have various modifications and variations. Any modifications, equivalent replacements, improvements and the like made within the spirit and principle of the present disclosure all fall within the scope of protection of the present disclosure.

What is claimed is:

1. A method for constructing a DNA library based on an MGI platform, comprising:
ligating a bubble adapter to a target sample to obtain a ligation product; and
performing library construction on the ligation product utilizing an amplification primer pair, obtaining an MGI platform based amplified library with dual indexes, wherein
the amplification primer pair has the dual indexes and comprises a 5′ end library index and a 3′ end library index;
the 5′ end library index is selected from indexes corresponding to any one of groups of 4-base-balanced phosphorylation ends in Table 1 or Table 2, and the 3′ end library index is selected from indexes corresponding to fixedly-matched non-phosphorylation ends corresponding to the 5′ end library index group in Table 1 or Table 2;
four-base-balance refers to balance of index sequences in groups of 4, the index sequences in groups of 4 are in Bits 1 to 10 of an index, with one for each base A, T, G and C;
the bubble adapter comprises a first adapter sequence and a second adapter sequence, the first adapter sequence is SEQ ID NO: 769, and the second adapter sequence is SEQ ID NO: 770;
each of the amplification primer pairs further comprises a 5′ end universal amplification sequence and a 3′ end universal amplification sequence, the 5′ end universal amplification sequence comprises a universal sequence located upstream of the 5′ end library index and a universal sequence located downstream of the 5′ end library index, and the 3′ end universal amplification sequence comprises a universal sequence located upstream of the 3′ end library index and a universal sequence located downstream of the 3′ end library index;
the universal sequence located upstream of the 5′ end library index is SEQ ID NO: 773, the universal sequence located downstream of the 5′ end library index is SEQ ID NO: 774; the universal sequence located upstream of the 3′ end library index is SEQ ID NO: 775, and the universal sequence located downstream of the 3′ end library index is SEQ ID NO: 771, and is used in combination with the first adapter sequence shown in SEQ ID NO: 769 and the second adapter sequence shown in SEQ ID NO: 770; and
TABLE 1

| | | | | | | | | | | | Index |
|---|---|---|---|---|---|---|---|---|---|---|---|
| | | | Index | | Index | | | | Index | | corres- |
| | | | corres- | | corres- | | | | corres- | | ponding |
| | | | ponding | | ponding | | | | ponding | | to non- |
| Bal- | | | to phos- | | to non- | Bal- | | | to phos- | | phos- |
| anced | | SEQ | phoryl- | SEQ | phosphoryl- | anced | | SEQ | phoryl- | SEQ | phoryl- |
| combi- | Num- | ID | ation | ID | ation | combin- | Num- | ID | ation | ID | ation |
| nation | ber | NO: | end | NO: | end | ation | ber | NO: | end | NO: | end |
| Group | M1- | 776 | TCACATT | 1159 | GATAGTAAC | Group | M1- | 968 | AGCTACT | 967 | ATGTTCAT |
| 1 | 001 | | GCT | | G | 49 | 193 | | CTG | | CC |
| | M1- | 777 | AATGGCG | 1158 | TGAGTGGCT | | M1- | 969 | CAACGT | 966 | TGACCACC |
| | 002 | | CTC | | A | | 194 | | GAGT | | AG |
| | M1- | 778 | GTCTCAA | 1157 | CCGTCATTA | | M1- | 970 | TCGATG | 965 | CACGGTTG |
| | 003 | | TGA | | C | | 195 | | CTAA | | GT |
| | M1- | 779 | CGGATGC | 1156 | ATCCACCGG | | M1- | 971 | GTTGCA | 964 | GCTAAGGA |
| | 004 | | AAG | | T | | 196 | | AGCC | | TA |
| Group | M1- | 780 | TCGCTTA | 1155 | GCAACTGT | Group | M1- | 972 | CGACATG | 963 | TCTGAAGA |
| 2 | 005 | | AGC | | GA | 50 | 197 | | TGT | | CA |
| | M1- | 781 | CGAGGC | 1154 | ATCCACCA | | M1- | 973 | GATGCGC | 962 | CACTGCAG |
| | 006 | | TTAG | | CC | | 198 | | ATA | | AG |
| | M1- | 782 | GTCTAAG | 1153 | CGTGTGAC | | M1- | 974 | TCGTGAT | 961 | GTAATGTCG |
| | 007 | | GCT | | AT | | 199 | | CAG | | T |
| | M1- | 783 | AATACGC | 1152 | TAGTGATG | | M1- | 975 | ATCATCA | 960 | AGGCCTCTT |
| | 008 | | CTA | | TG | | 200 | | GCC | | C |
| Group | M1- | 784 | AAGCCTA | 1151 | GCTTGTTCA | Group | M1- | 976 | TCAATG | 959 | TAGCAAGT |
| 3 | 009 | | TTG | | G | 51 | 201 | | GCGG | | TG |
| | M1- | 785 | CGCTACT | 1150 | AACAAGCA | | M1- | 977 | CAGTAA | 958 | CGCTTCTC |
| | 010 | | GCA | | CT | | 202 | | CTCT | | CT |
| | M1- | 786 | TCAAGAG | 1149 | TTGCCAGTG | | M1- | 978 | GTTGCC | 957 | ACAAGTAA |
| | 011 | | CAT | | A | | 203 | | TGAC | | GC |
| | M1- | 787 | GTTGTGC | 1148 | CGAGTCAGT | | M1- | 979 | AGCCGT | 956 | GTTGCGCG |
| | 012 | | AGC | | C | | 204 | | AATA | | AA |
| Group | M1- | 788 | AGACAG | 1147 | TCGCGATG | Group | M1- | 980 | CTAATAG | 955 | AACATCGT |
| 4 | 013 | | GAAT | | TC | 52 | 205 | | GCT | | AG |
| | M1- | 789 | CCTTGCC | 1146 | AGTGACAC | | M1- | 981 | TCTCCTC | 954 | TGATCGAG |
| | 014 | | GTA | | CA | | 206 | | CAC | | GC |
| | M1- | 790 | GTCATTA | 1145 | GACATTCA | | M1- | 982 | AGCTGC | 953 | GTTGGACA |
| | 015 | | CGG | | AG | | 207 | | ATGA | | TA |
| | M1- | 791 | TAGGCAT | 1144 | CTATCGGT | | M1- | 983 | GAGGAG | 952 | CCGCATTC |
| | 016 | | TCC | | GT | | 208 | | TATG | | CT |
| Group | M1- | 792 | CATATCA | 1143 | TACTTCGG | Group | M1- | 984 | ACCACG | 951 | AGAACCTA |
| 5 | 017 | | TCG | | AT | 53 | 209 | | TAGC | | CA |
| | M1- | 793 | GCACAAC | 1142 | CGTGCGATC | | M1- | 985 | GAATGCA | 950 | TCGTGTCCA |
| | 018 | | AAT | | A | | 210 | | GTA | | T |
| | M1- | 794 | TTGTCGT | 1141 | ACACAACA | | M1- | 986 | TTGGTAG | 949 | CTCGTAGGT |
| | 019 | | GGC | | TG | | 211 | | CCT | | C |
| | M1- | 795 | AGCGGT | 1140 | GTGAGTTC | | M1- | 987 | CGTCATC | 948 | GATCAGATG |
| | 020 | | GCTA | | GC | | 212 | | TAG | | G |
| Group | M1- | 796 | AGCCAG | 1139 | CCGATGAC | Group | M1- | 988 | TCAATGA | 947 | GTGTGTTGC |
| 6 | 021 | | TAGG | | GT | 54 | 213 | | GGT | | G |
| | M1- | 797 | GTAAGTG | 1138 | TTATCTCGA | | M1- | 989 | AGCGAA | 946 | AGTAAGCC |
| | 022 | | TAC | | G | | 214 | | GCTG | | AA |
| | M1- | 798 | TAGTCAC | 1137 | AGCCGATA | | M1- | 990 | CTGTCTT | 945 | CAACTAGT |
| | 023 | | GTT | | CC | | 215 | | AAC | | GT |
| | M1- | 799 | CCTGTCA | 1136 | GATGACGT | | M1- | 991 | GATCGCC | 944 | TCCGCCAAT |
| | 024 | | CCA | | TA | | 216 | | TCA | | C |
| Group | M1- | 800 | ATCGTGG | 1135 | GACGCGGT | Group | M1- | 992 | GTTCCG | 943 | TGGCGATA |
| 7 | 025 | | ATG | | AT | 55 | 217 | | AATG | | CA |
| | M1- | 801 | TGGAGAT | 1134 | ACGAGACG | | M1- | 993 | TACGTAC | 942 | GCTACCAC |
| | 026 | | CGA | | TC | | 218 | | GCA | | GT |
| | M1- | 802 | CCTCACA | 1133 | CTACTCAAG | | M1- | 994 | CGGAGTT | 941 | AACGTTGTT |
| | 027 | | GAT | | A | | 219 | | CAC | | C |
| | M1- | 803 | GAATCTC | 1132 | TGTTATTCC | | M1- | 995 | ACATACG | 940 | CTATAGCG |
| | 028 | | TCC | | G | | 220 | | TGT | | AG |
| Group | M1- | 804 | GCAGACT | 1131 | CCGTCACTG | Group | M1- | 996 | CTGAAGA | 939 | CGCTTGAAT |
| 8 | 029 | | GAC | | A | 56 | 221 | | GAT | | T |
| | M1- | 805 | CTCATTA | 1130 | TGACGCAA | | M1- | 997 | GAAGCCT | 938 | ATGACTGCC |
| | 030 | | ACG | | CT | | 222 | | CCA | | G |
| | M1- | 806 | TGGTGA | 1129 | GTTGTTGC | | M1- | 998 | TGTCTTC | 937 | TAACACCG |
| | 031 | | GCTT | | TC | | 223 | | ATC | | GC |
| | M1- | 807 | AATCCGC | 1128 | AACAAGTG | | M1- | 999 | ACCTGAG | 936 | GCTGGATTA |
| | 032 | | TGA | | AG | | 224 | | TGG | | A |
| Group | M1- | 808 | TCGCATC | 1127 | TGGTTGGA | Group | M1- | 1000 | GTACGTC | 935 | AGACGGTC |
| 9 | 033 | | AAC | | AT | 57 | 225 | | CTT | | AA |
| | M1- | 809 | AGAACA | 1126 | GAACGTTC | | M1- | 1001 | AACTAG | 934 | TAGATCCA |
| | 034 | | GTGA | | GG | | 226 | | GTCA | | CC |
| | M1- | 810 | CATGTCT | 1125 | ATCGCACG | | M1- | 1002 | TGTGTCA | 933 | GCCTCTATG |
| | 035 | | CCT | | CA | | 227 | | GGC | | G |
| | M1- | 811 | GTCTGG | 1124 | CCTAACATT | | M1- | 1003 | CCGACA | 932 | CTTGAAGG |
| | 036 | | AGTG | | C | | 228 | | TAAG | | TT |
| Group | M1- | 812 | GAGGTCT | 1123 | CGTGTGGAT | Group | M1- | 1004 | TCCACAC | 931 | ATAACGGT |
| 10 | 037 | | GTG | | G | 58 | 229 | | GTC | | GG |
| | M1- | 813 | CTATAGA | 1122 | TTACGTCGG | | M1- | 1005 | CGGCAC | 930 | TGCCACTG |
| | 038 | | CGT | | T | | 230 | | ATGA | | TA |
| | M1- | 814 | TGCAGTG | 1121 | ACGAACATA | | M1- | 1006 | GTATGTT | 929 | CATGTACC |
| | 039 | | ACC | | C | | 231 | | CCT | | AC |
| | M1- | 815 | ACTCCAC | 1120 | GACTCATCC | | M1- | 1007 | AATGTG | 928 | GCGTGTAA |
| | 040 | | TAA | | A | | 232 | | GAAG | | CT |
| Group | M1- | 816 | GCGAAG | 1119 | TGCCTGAC | Group | M1- | 1008 | AGACAG | 927 | TGTAACGC |
| 11 | 041 | | TAGG | | TC | 59 | 233 | | ACGT | | TG |
| | M1- | 817 | TGCCTAA | 1118 | CATTATCGC | | M1- | 1009 | TTCTGT | 926 | GCCGTTAT |
| | 042 | | CCT | | T | | 234 | | GGAG | | GC |
| | M1- | 818 | AATGGTC | 1117 | GTAAGCGA | | M1- | 1010 | CAGGTCT | 925 | CTATCACAC |
| | 043 | | TAC | | GG | | 235 | | TCC | | T |
| | M1- | 819 | CTATCCG | 1116 | ACGGCATT | | M1- | 1011 | GCTACAC | 924 | AAGCGGTG |
| | 044 | | GTA | | AA | | 236 | | ATA | | AA |
| Group | M1- | 820 | TACGCTT | 1115 | GACACTGCC | Group | M1- | 1012 | CTAGCG | 923 | GACCTGTC |
| 12 | 045 | | CAG | | A | 60 | 237 | | ACAC | | CT |
| | M1- | 821 | CGGAGCA | 1114 | CCGTAGCAT | | M1- | 1013 | AGGCAT | 922 | AGTAATCG |
| | 046 | | TCT | | G | | 238 | | TACT | | AC |
| | M1- | 822 | GTACTAG | 1113 | ATTCTATGG | | M1- | 1014 | GACATC | 921 | CTGTGCAT |
| | 047 | | ATC | | C | | 239 | | CGGA | | TA |
| | M1- | 823 | ACTTAGC | 1112 | TGAGGCATA | | M1- | 1015 | TCTTGAG | 920 | TCAGCAGA |
| | 048 | | GGA | | T | | 240 | | TTG | | GG |
| Group | M1- | 824 | ATCACTC | 1111 | ATACGCGC | Group | M1- | 1016 | ACAACA | 919 | ACATGAAT |
| 13 | 049 | | CAT | | CA | 61 | 241 | | GAAG | | GC |
| | M1- | 825 | GATCGCA | 1110 | TCTTAGTG | | M1- | 1017 | CGCTTG | 918 | TGCCTCCA |
| | 050 | | GTG | | AC | | 242 | | TGGA | | CT |
| | M1- | 826 | CCGGAAT | 1109 | GAGGTACA | | M1- | 1018 | GTTGGT | 917 | GATACTTG |
| | 051 | | TCC | | TT | | 243 | | CTCC | | TG |
| | M1- | 827 | TGATTGG | 1108 | CGCACTAT | | M1- | 1019 | TAGCAC | 916 | CTGGAGGC |
| | 052 | | AGA | | GG | | 244 | | ACTT | | AA |
| Group | M1- | 828 | CACAAGG | 1107 | TGGCTCGCT | Group | M1- | 1020 | GCTTGC | 915 | TCGCGGAT |
| 14 | 053 | | TCG | | T | 62 | 245 | | AATA | | CA |
| | M1- | 829 | TCTCGCA | 1106 | GTCACTTAA | | M1- | 1021 | TGGACG | 914 | CATTCTCA |
| | 054 | | GGA | | G | | 246 | | TGCT | | TC |
| | M1- | 830 | GTGGTAT | 1105 | CAATAGAGG | | M1- | 1022 | AACGAA | 913 | GTAGACGG |
| | 055 | | CAT | | C | | 247 | | CCGG | | AG |
| | M1- | 831 | AGATCTC | 1104 | ACTGGACTC | | M1- | 1023 | CTACTTG | 912 | AGCATATC |
| | 056 | | ATC | | A | | 248 | | TAC | | GT |
| Group | M1- | 832 | GATGGAG | 1103 | CCGATCATC | Group | M1- | 1024 | CGGTGAG | 911 | TTGTCGGC |
| 15 | 057 | | ATT | | C | 63 | 249 | | TGA | | AC |
| | M1- | 833 | CTCATTCT | 1102 | GTTCGATAA | | M1- | 1025 | GCAATGC | 910 | GACGAACT |
| | 058 | | GC | | G | | 250 | | ATT | | CG |
| | M1- | 834 | TGGCAGA | 1101 | AGCTCGGCT | | M1- | 1026 | TTCCATT | 909 | AGTAGCTA |
| | 059 | | CAA | | T | | 251 | | CAC | | GT |
| | M1- | 835 | ACATCCT | 1100 | TAAGATCGG | | M1- | 1027 | AATGCCA | 908 | CCACTTAGT |
| | 060 | | GCG | | A | | 252 | | GCG | | A |
| Group | M1- | 836 | ACGTCG | 1099 | CCAGTTAAT | Group | M1- | 1028 | TACTCTT | 907 | TAATAAGC |
| 16 | 061 | | CAGA | | C | 64 | 253 | | CTC | | CG |
| | M1- | 837 | TGTATAG | 1098 | GTTCGATT | | M1- | 1029 | AGGAAG | 906 | CGTCGTCA |
| | 062 | | GCT | | GT | | 254 | | GTAA | | AC |
| | M1- | 838 | CAACACA | 1097 | AGGTACGC | | M1- | 1030 | GTTGGA | 905 | ATCGTGAG |
| | 063 | | TTG | | AG | | 255 | | CGGT | | TT |
| | M1- | 839 | GTCGGTT | 1096 | TACACGCGC | | M1- | 1031 | CCACTC | 904 | GCGACCTT |
| | 064 | | CAC | | A | | 256 | | AACG | | GA |
| Group | M1- | 840 | AGCCATA | 1095 | TGCTATGCA | Group | M1- | 1032 | AATGACT | 903 | GAACAGGA |
| 17 | 065 | | AGC | | G | 65 | 257 | | GGT | | CC |
| | M1- | 841 | GTATTCC | 1094 | GTTCGCTGT | | M1- | 1033 | GTGCCA | 902 | TGTGCTCC |
| | 066 | | GAG | | A | | 258 | | CAAC | | TA |
| | M1- | 842 | CATAGGT | 1093 | CCAGTACTG | | M1- | 1034 | CGAATG | 901 | CTCAGCTT |
| | 067 | | TCA | | C | | 259 | | ATCG | | AG |
| | M1- | 843 | TCGGCAG | 1092 | AAGACGAA | | M1- | 1035 | TCCTGT | 900 | ACGTTAAG |
| | 068 | | CTT | | CT | | 260 | | GCTA | | GT |
| Group | M1- | 844 | GTTCGGT | 1091 | AGAACGCG | Group | M1- | 1036 | TGTGAAT | 899 | CGTAGAGT |
| 18 | 069 | | CCT | | TC | 66 | 261 | | TGG | | CA |
| | M1- | 845 | TGGATTG | 1090 | TTCTGCTT | | M1- | 1037 | AACCGGC | 898 | GACCAGTA |
| | 070 | | TAG | | GT | | 262 | | CTT | | AG |
| | M1- | 846 | CCAGAA | 1089 | GCGGATAC | | M1- | 1038 | CTATCCG | 897 | ACGGTTCG |
| | 071 | | CGTC | | AG | | 263 | | ACC | | TC |
| | M1- | 847 | AACTCCA | 1088 | CATCTAGAC | | M1- | 1039 | GCGATTA | 896 | TTATCCACG |
| | 072 | | AGA | | A | | 264 | | GAA | | T |
| Group | M1- | 848 | CGTACAC | 1087 | AACAAGGC | Group | M1- | 1040 | CGTAAC | 895 | GCAGGATG |
| 19 | 073 | | TGG | | TT | 67 | 265 | | CGCA | | GT |
| | M1- | 849 | GCACACA | 1086 | TGATGTCGA | | M1- | 1041 | GACTGA | 894 | AGTATCAC |
| | 074 | | GCA | | A | | 266 | | TAAC | | TC |
| | M1- | 850 | ATGGTGT | 1085 | GCTCCATTC | | M1- | 1042 | ATGCTTA | 893 | TTGTCTGT |
| | 075 | | ATC | | C | | 267 | | CTG | | AG |
| | M1- | 851 | TACTGTG | 1084 | CTGGTCAAG | | M1- | 1043 | TCAGCGG | 892 | CACCAGCA |
| | 076 | | CAT | | G | | 268 | | TGT | | CA |
| Group | M1- | 852 | CGGCAAT | 1083 | GATATGCT | Group | M1- | 1044 | ACATAAC | 891 | CTTGTATA |
| 20 | 077 | | CAG | | GA | 68 | 269 | | ACC | | CC |
| | M1- | 853 | GTAGTTC | 1082 | CTGGATTAC | | M1- | 1045 | CACCTG | 890 | TGCTCTCT |
| | 078 | | GGA | | T | | 270 | | AGGT | | GG |
| | M1- | 854 | TACAGGA | 1081 | AGACGAGG | | M1- | 1046 | GTGACT | 889 | GAACGGAC |
| | 079 | | ACT | | TC | | 271 | | GTAA | | TA |
| | M1- | 855 | ACTTCCG | 1080 | TCCTCCACA | | M1- | 1047 | TGTGGC | 888 | ACGAACGG |
| | 080 | | TTC | | G | | 272 | | TCTG | | AT |
| Group | M1- | 856 | TGCTCCA | 1079 | CAAGACTC | Group | M1- | 1048 | CTCAGA | 887 | GCACCGCT |
| 21 | 081 | | CGA | | AA | 69 | 273 | | CTCT | | AT |
| | M1- | 857 | AATCAAG | 1078 | GTGTCTGA | | M1- | 1049 | ACACATG | 886 | TTGAGCGA |
| | 082 | | GTC | | CC | | 274 | | CTA | | CG |
| | M1- | 858 | GTAGGTC | 1077 | ACCAGACG | | M1- | 1050 | TGTGCCT | 885 | CGCTTAAGT |
| | 083 | | AAT | | GT | | 275 | | AAG | | A |
| | M1- | 859 | CCGATGT | 1076 | TGTCTGATT | | M1- | 1051 | GAGTTGA | 884 | AATGATTCG |
| | 084 | | TCG | | G | | 276 | | GGC | | C |
| Group | M1- | 860 | GACGTG | 1075 | AGCGTATTA | Group | M1- | 1052 | AGCCGTT | 883 | TGGCACTG |
| 22 | 085 | | TGCA | | C | 70 | 277 | | CTC | | AT |
| | M1- | 861 | TCGCCAC | 1074 | CTGTAGCG | | M1- | 1053 | TTGTTGG | 882 | GTCACGAC |
| | 086 | | TTC | | CT | | 278 | | TCT | | TG |
| | M1- | 862 | ATAAGCA | 1073 | TCAAGCAC | | M1- | 1054 | CAAGAC | 881 | CATTGAGA |
| | 087 | | CGT | | GG | | 279 | | AAGA | | GC |
| | M1- | 863 | CGTTATG | 1072 | GATCCTGA | | M1- | 1055 | GCTACAC | 880 | ACAGTTCTC |
| | 088 | | AAG | | TA | | 280 | | GAG | | A |
| Group | M1- | 864 | AATAGAG | 1071 | TGCGCAGA | Group | M1- | 1056 | GTGACG | 879 | TGGTATTC |
| 23 | 089 | | CCA | | AG | 71 | 281 | | CGAT | | TC |
| | M1- | 865 | GTACCTC | 1070 | GCTTGGTC | | M1- | 1057 | AACCTCT | 878 | ATAAGCGG |
| | 090 | | GAC | | GA | | 282 | | CTG | | AG |
| | M1- | 866 | CCGGTG | 1069 | CTAATCCGT | | M1- | 1058 | TCTTGAG | 877 | GCCGTGAA |
| | 091 | | ATTG | | T | | 283 | | AGA | | CA |
| | M1- | 867 | TGCTACT | 1068 | AAGCATATC | | M1- | 1059 | CGAGAT | 876 | CATCCACT |
| | 092 | | AGT | | C | | 284 | | ATCC | | GT |
| Group | M1- | 868 | GATCCG | 1067 | TAGCCTAA | Group | M1- | 1060 | GAAGGAT | 875 | TCTCTCAAC |
| 24 | 093 | | GACT | | GA | 72 | 285 | | TCA | | A |
| | M1- | 869 | TTAGGCA | 1066 | GCCTGACG | | M1- | 1061 | TCGCCTG | 874 | AGCGGTGG |
| | 094 | | CAA | | TT | | 286 | | GTT | | AT |
| | M1- | 870 | AGCATTC | 1065 | CTTATGGC | | M1- | 1062 | AGTAACA | 873 | CAGACATCT |
| | 095 | | TTG | | CG | | 287 | | CGG | | G |
| | M1- | 871 | CCGTAAT | 1064 | AGAGACTT | | M1- | 1063 | CTCTTGC | 872 | GTATAGCTG |
| | 096 | | GGC | | AC | | 288 | | AAC | | C |
| Group | M1- | 872 | GTATAGC | 1063 | CTCTTGCAA | Group | M1- | 1064 | AGAGAC | 871 | CCGTAATG |
| 25 | 097 | | TGC | | C | 73 | 289 | | TTAC | | GC |
| | M1- | 873 | CAGACAT | 1062 | AGTAACAC | | M1- | 1065 | CTTATGG | 870 | AGCATTCT |
| | 098 | | CTG | | GG | | 290 | | CCG | | TG |
| | M1- | 874 | AGCGGTG | 1061 | TCGCCTGGT | | M1- | 1066 | GCCTGA | 869 | TTAGGCAC |
| | 099 | | GAT | | T | | 291 | | CGTT | | AA |
| | M1- | 875 | TCTCTCA | 1060 | GAAGGATT | | M1- | 1067 | TAGCCTA | 868 | GATCCGGA |
| | 100 | | ACA | | CA | | 292 | | AGA | | CT |
| Group | M1- | 876 | CATCCAC | 1059 | CGAGATAT | Group | M1- | 1068 | AAGCATA | 867 | TGCTACTA |
| 26 | 101 | | TGT | | CC | 74 | 293 | | TCC | | GT |
| | M1- | 877 | GCCGTGA | 1058 | TCTTGAGAG | | M1- | 1069 | CTAATCC | 866 | CCGGTGAT |
| | 102 | | ACA | | A | | 294 | | GTT | | TG |
| | M1- | 878 | ATAAGCG | 1057 | AACCTCTCT | | M1- | 1070 | GCTTGG | 865 | GTACCTCG |
| | 103 | | GAG | | G | | 295 | | TCGA | | AC |
| | M1- | 879 | TGGTATT | 1056 | GTGACGCG | | M1- | 1071 | TGCGCAG | 864 | AATAGAGC |
| | 104 | | CTC | | AT | | 296 | | AAG | | CA |
| Group | M1- | 880 | ACAGTTC | 1055 | GCTACACG | Group | M1- | 1072 | GATCCT | 863 | CGTTATGA |
| 27 | 105 | | TCA | | AG | 75 | 297 | | GATA | | AG |
| | M1- | 881 | CATTGAG | 1054 | CAAGACAA | | M1- | 1073 | TCAAGC | 862 | ATAAGCAC |
| | 106 | | AGC | | GA | | 298 | | ACGG | | GT |
| | M1- | 882 | GTCACG | 1053 | TTGTTGGT | | M1- | 1074 | CTGTAG | 861 | TCGCCACT |
| | 107 | | ACTG | | CT | | 299 | | CGCT | | TC |
| | M1- | 883 | TGGCACT | 1052 | AGCCGTTC | | M1- | 1075 | AGCGTAT | 860 | GACGTGTG |
| | 108 | | GAT | | TC | | 300 | | TAC | | CA |
| Group | M1- | 884 | AATGATT | 1051 | GAGTTGAG | Group | M1- | 1076 | TGTCTG | 859 | CCGATGTT |
| 28 | 109 | | CGC | | GC | 76 | 301 | | ATTG | | CG |
| | M1- | 885 | CGCTTAA | 1050 | TGTGCCTA | | M1- | 1077 | ACCAGA | 858 | GTAGGTCA |
| | 110 | | GTA | | AG | | 302 | | CGGT | | AT |
| | M1- | 886 | TTGAGC | 1049 | ACACATGC | | M1- | 1078 | GTGTCT | 857 | AATCAAGG |
| | 111 | | GACG | | TA | | 303 | | GACC | | TC |
| | M1- | 887 | GCACCGC | 1048 | CTCAGACTC | | M1- | 1079 | CAAGAC | 856 | TGCTCCAC |
| | 112 | | TAT | | T | | 304 | | TCAA | | GA |
| Group | M1- | 888 | ACGAAC | 1047 | TGTGGCTC | Group | M1- | 1080 | TCCTCCA | 855 | ACTTCCGTT |
| 29 | 113 | | GGAT | | TG | 77 | 305 | | CAG | | C |
| | M1- | 889 | GAACGG | 1046 | GTGACTGT | | M1- | 1081 | AGACGA | 854 | TACAGGAA |
| | 114 | | ACTA | | AA | | 306 | | GGTC | | CT |
| | M1- | 890 | TGCTCTC | 1045 | CACCTGAG | | M1- | 1082 | CTGGATT | 853 | GTAGTTCG |
| | 115 | | TGG | | GT | | 307 | | ACT | | GA |
| | M1- | 891 | CTTGTAT | 1044 | ACATAACA | | M1- | 1083 | GATATGC | 852 | CGGCAATC |
| | 116 | | ACC | | CC | | 308 | | TGA | | AG |
| Group | M1- | 892 | CACCAGC | 1043 | TCAGCGGTG | Group | M1- | 1084 | CTGGTCA | 851 | TACTGTGCA |
| 30 | 117 | | ACA | | T | 78 | 309 | | AGG | | T |
| | M1- | 893 | TTGTCTG | 1042 | ATGCTTACT | | M1- | 1085 | GCTCCAT | 850 | ATGGTGTAT |
| | 118 | | TAG | | G | | 310 | | TCC | | C |
| | M1- | 894 | AGTATCA | 1041 | GACTGATAA | | M1- | 1086 | TGATGTC | 849 | GCACACAG |
| | 119 | | CTC | | C | | 311 | | GAA | | CA |
| | M1- | 895 | GCAGGA | 1040 | CGTAACCG | | M1- | 1087 | AACAAG | 848 | CGTACACT |
| | 120 | | TGGT | | CA | | 312 | | GCTT | | GG |
| Group | M1- | 896 | TTATCCA | 1039 | GCGATTAG | Group | M1- | 1088 | CATCTAG | 847 | AACTCCAA |
| 31 | 121 | | CGT | | AA | 79 | 313 | | ACA | | GA |
| | M1- | 897 | ACGGTTC | 1038 | CTATCCGA | | M1- | 1089 | GCGGAT | 846 | CCAGAACG |
| | 122 | | GTC | | CC | | 314 | | ACAG | | TC |
| | M1- | 898 | GACCAG | 1037 | AACCGGCC | | M1- | 1090 | TTCTGCT | 845 | TGGATTGT |
| | 123 | | TAAG | | TT | | 315 | | TGT | | AG |
| | M1- | 899 | CGTAGA | 1036 | TGTGAATT | | M1- | 1091 | AGAACG | 844 | GTTCGGTC |
| | 124 | | GTCA | | GG | | 316 | | CGTC | | CT |
| Group | M1- | 900 | ACGTTAA | 1035 | TCCTGTGCT | Group | M1- | 1092 | AAGACG | 843 | TCGGCAGC |
| 32 | 125 | | GGT | | A | 80 | 317 | | AACT | | TT |
| | M1- | 901 | CTCAGCT | 1034 | CGAATGATC | | M1- | 1093 | CCAGTAC | 842 | CATAGGTTC |
| | 126 | | TAG | | G | | 318 | | TGC | | A |
| | M1- | 902 | TGTGCTC | 1033 | GTGCCACAA | | M1- | 1094 | GTTCGC | 841 | GTATTCCG |
| | 127 | | CTA | | C | | 319 | | TGTA | | AG |
| | M1- | 903 | GAACAGG | 1032 | AATGACTGG | | M1- | 1095 | TGCTATG | 840 | AGCCATAA |
| | 128 | | ACC | | T | | 320 | | CAG | | GC |
| Group | M1- | 904 | GCGACCT | 1031 | CCACTCAAC | Group | M1- | 1096 | TACACGC | 839 | GTCGGTTC |
| 33 | 129 | | TGA | | G | 81 | 321 | | GCA | | AC |
| | M1- | 905 | ATCGTGA | 1030 | GTTGGACG | | M1- | 1097 | AGGTACG | 838 | CAACACATT |
| | 130 | | GTT | | GT | | 322 | | CAG | | G |
| | M1- | 906 | CGTCGTC | 1029 | AGGAAGGT | | M1- | 1098 | GTTCGAT | 837 | TGTATAGGC |
| | 131 | | AAC | | AA | | 323 | | TGT | | T |
| | M1- | 907 | TAATAAG | 1028 | TACTCTTCT | | M1- | 1099 | CCAGTTA | 836 | ACGTCGCA |
| | 132 | | CCG | | C | | 324 | | ATC | | GA |
| Group | M1- | 908 | CCACTTA | 1027 | AATGCCAG | Group | M1- | 1100 | TAAGATC | 835 | ACATCCTG |
| 34 | 133 | | GTA | | CG | 82 | 325 | | GGA | | CG |
| | M1- | 909 | AGTAGCT | 1026 | TTCCATTCA | | M1- | 1101 | AGCTCG | 834 | TGGCAGAC |
| | 134 | | AGT | | C | | 326 | | GCTT | | AA |
| | M1- | 910 | GACGAA | 1025 | GCAATGCA | | M1- | 1102 | GTTCGAT | 833 | CTCATTCT |
| | 135 | | CTCG | | TT | | 327 | | AAG | | GC |
| | M1- | 911 | TTGTCGG | 1024 | CGGTGAGT | | M1- | 1103 | CCGATC | 832 | GATGGAGA |
| | 136 | | CAC | | GA | | 328 | | ATCC | | TT |
| Group | M1- | 912 | AGCATAT | 1023 | CTACTTGTA | Group | M1- | 1104 | ACTGGA | 831 | AGATCTCA |
| 35 | 137 | | CGT | | C | 83 | 329 | | CTCA | | TC |
| | M1- | 913 | GTAGAC | 1022 | AACGAACC | | M1- | 1105 | CAATAGA | 830 | GTGGTATC |
| | 138 | | GGAG | | GG | | 330 | | GGC | | AT |
| | M1- | 914 | CATTCTC | 1021 | TGGACGTG | | M1- | 1106 | GTCACT | 829 | TCTCGCAG |
| | 139 | | ATC | | CT | | 331 | | TAAG | | GA |
| | M1- | 915 | TCGCGG | 1020 | GCTTGCAA | | M1- | 1107 | TGGCTC | 828 | CACAAGGT |
| | 140 | | ATCA | | TA | | 332 | | GCTT | | CG |
| Group | M1- | 916 | CTGGAG | 1019 | TAGCACAC | Group | M1- | 1108 | CGCACTA | 827 | TGATTGGA |
| 36 | 141 | | GCAA | | TT | 84 | 333 | | TGG | | GA |
| | M1- | 917 | GATACTT | 1018 | GTTGGTCT | | M1- | 1109 | GAGGTAC | 826 | CCGGAATTC |
| | 142 | | GTG | | CC | | 334 | | ATT | | C |
| | M1- | 918 | TGCCTCC | 1017 | CGCTTGTG | | M1- | 1110 | TCTTAGT | 825 | GATCGCAG |
| | 143 | | ACT | | GA | | 335 | | GAC | | TG |
| | M1- | 919 | ACATGAA | 1016 | ACAACAGA | | M1- | 1111 | ATACGCG | 824 | ATCACTCCA |
| | 144 | | TGC | | AG | | 336 | | CCA | | T |
| Group | M1- | 920 | TCAGCA | 1015 | TCTTGAGT | Group | M1- | 1112 | TGAGGC | 823 | ACTTAGCG |
| 37 | 145 | | GAGG | | TG | 85 | 337 | | ATAT | | GA |
| | M1- | 921 | CTGTGCA | 1014 | GACATCCG | | M1- | 1113 | ATTCTAT | 822 | GTACTAGA |
| | 146 | | TTA | | GA | | 338 | | GGC | | TC |
| | M1- | 922 | AGTAATC | 1013 | AGGCATTA | | M1- | 1114 | CCGTAG | 821 | CGGAGCAT |
| | 147 | | GAC | | CT | | 339 | | CATG | | CT |
| | M1- | 923 | GACCTGT | 1012 | CTAGCGAC | | M1- | 1115 | GACACT | 820 | TACGCTTC |
| | 148 | | CCT | | AC | | 340 | | GCCA | | AG |
| Group | M1- | 924 | AAGCGGT | 1011 | GCTACACAT | Group | M1- | 1116 | ACGGCAT | 819 | CTATCCGGT |
| 38 | 149 | | GAA | | A | 86 | 341 | | TAA | | A |
| | M1- | 925 | CTATCAC | 1010 | CAGGTCTT | | M1- | 1117 | GTAAGCG | 818 | AATGGTCTA |
| | 150 | | ACT | | CC | | 342 | | AGG | | C |
| | M1- | 926 | GCCGTTA | 1009 | TTCTGTGGA | | M1- | 1118 | CATTATC | 817 | TGCCTAACC |
| | 151 | | TGC | | G | | 343 | | GCT | | T |
| | M1- | 927 | TGTAACG | 1008 | AGACAGAC | | M1- | 1119 | TGCCTGA | 816 | GCGAAGTA |
| | 152 | | CTG | | GT | | 344 | | CTC | | GG |
| Group | M1- | 928 | GCGTGTA | 1007 | AATGTGGAA | Group | M1- | 1120 | GACTCAT | 815 | ACTCCACT |
| 39 | 153 | | ACT | | G | 87 | 345 | | CCA | | AA |
| | M1- | 929 | CATGTAC | 1006 | GTATGTTCC | | M1- | 1121 | ACGAAC | 814 | TGCAGTGA |
| | 154 | | CAC | | T | | 346 | | ATAC | | CC |
| | M1- | 930 | TGCCACT | 1005 | CGGCACATG | | M1- | 1122 | TTACGTC | 813 | CTATAGAC |
| | 155 | | GTA | | A | | 347 | | GGT | | GT |
| | M1- | 931 | ATAACGG | 1004 | TCCACACGT | | M1- | 1123 | CGTGTGG | 812 | GAGGTCTG |
| | 156 | | TGG | | C | | 348 | | ATG | | TG |
| Group | M1- | 932 | CTTGAAG | 1003 | CCGACATA | Group | M1- | 1124 | CCTAACA | 811 | GTCTGGAG |
| 40 | 157 | | GTT | | AG | 88 | 349 | | TTC | | TG |
| | M1- | 933 | GCCTCTA | 1002 | TGTGTCAGG | | M1- | 1125 | ATCGCA | 810 | CATGTCTC |
| | 158 | | TGG | | C | | 350 | | CGCA | | CT |
| | M1- | 934 | TAGATCC | 1001 | AACTAGGT | | M1- | 1126 | GAACGTT | 809 | AGAACAGT |
| | 159 | | ACC | | CA | | 351 | | CGG | | GA |
| | M1- | 935 | AGACGG | 1000 | GTACGTCC | | M1- | 1127 | TGGTTG | 808 | TCGCATCA |
| | 160 | | TCAA | | TT | | 352 | | GAAT | | AC |
| Group | M1- | 936 | GCTGGAT | 999 | ACCTGAGTG | Group | M1- | 1128 | AACAAG | 807 | AATCCGCT |
| 41 | 161 | | TAA | | G | 89 | 353 | | TGAG | | GA |
| | M1- | 937 | TAACACC | 998 | TGTCTTCAT | | M1- | 1129 | GTTGTT | 806 | TGGTGAGC |
| | 162 | | GGC | | C | | 354 | | GCTC | | TT |
| | M1- | 938 | ATGACTG | 997 | GAAGCCTC | | M1- | 1130 | TGACGC | 805 | CTCATTAA |
| | 163 | | CCG | | CA | | 355 | | AACT | | CG |
| | M1- | 939 | CGCTTGA | 996 | CTGAAGAG | | M1- | 1131 | CCGTCAC | 804 | GCAGACTG |
| | 164 | | ATT | | AT | | 356 | | TGA | | AC |
| Group | M1- | 940 | CTATAGC | 995 | ACATACGT | Group | M1- | 1132 | TGTTATT | 803 | GAATCTCT |
| 42 | 165 | | GAG | | GT | 90 | 357 | | CCG | | CC |
| | M1- | 941 | AACGTTG | 994 | CGGAGTTCA | | M1- | 1133 | CTACTCA | 802 | CCTCACAG |
| | 166 | | TTC | | C | | 358 | | AGA | | AT |
| | M1- | 942 | GCTACCA | 993 | TACGTACGC | | M1- | 1134 | ACGAGA | 801 | TGGAGATC |
| | 167 | | CGT | | A | | 359 | | CGTC | | GA |
| | M1- | 943 | TGGCGAT | 992 | GTTCCGAA | | M1- | 1135 | GACGCG | 800 | ATCGTGGA |
| | 168 | | ACA | | TG | | 360 | | GTAT | | TG |
| Group | M1- | 944 | TCCGCCA | 991 | GATCGCCT | Group | M1- | 1136 | GATGACG | 799 | CCTGTCACC |
| 43 | 169 | | ATC | | CA | 91 | 361 | | TTA | | A |
| | M1- | 945 | CAACTAG | 990 | CTGTCTTA | | M1- | 1137 | AGCCGAT | 798 | TAGTCACGT |
| | 170 | | TGT | | AC | | 362 | | ACC | | T |
| | M1- | 946 | AGTAAGC | 989 | AGCGAAGC | | M1- | 1138 | TTATCTC | 797 | GTAAGTGTA |
| | 171 | | CAA | | TG | | 363 | | GAG | | C |
| | M1- | 947 | GTGTGTT | 988 | TCAATGAG | | M1- | 1139 | CCGATGA | 796 | AGCCAGTA |
| | 172 | | GCG | | GT | | 364 | | CGT | | GG |
| Group | M1- | 948 | GATCAGA | 987 | CGTCATCTA | Group | M1- | 1140 | GTGAGT | 795 | AGCGGTGC |
| 44 | 173 | | TGG | | G | 92 | 365 | | TCGC | | TA |
| | M1- | 949 | CTCGTAG | 986 | TTGGTAGC | | M1- | 1141 | ACACAA | 794 | TTGTCGTG |
| | 174 | | GTC | | CT | | 366 | | CATG | | GC |
| | M1- | 950 | TCGTGTC | 985 | GAATGCAG | | M1- | 1142 | CGTGCG | 793 | GCACAACA |
| | 175 | | CAT | | TA | | 367 | | ATCA | | AT |
| | M1- | 951 | AGAACCT | 984 | ACCACGTA | | M1- | 1143 | TACTTCG | 792 | CATATCATC |
| | 176 | | ACA | | GC | | 368 | | GAT | | G |
| Group | M1- | 952 | CCGCATT | 983 | GAGGAGTAT | Group | M1- | 1144 | CTATCGG | 791 | TAGGCATTC |
| 45 | 177 | | CCT | | G | 93 | 369 | | TGT | | C |
| | M1- | 953 | GTTGGAC | 982 | AGCTGCATG | | M1- | 1145 | GACATTC | 790 | GTCATTACG |
| | 178 | | ATA | | A | | 370 | | AAG | | G |
| | M1- | 954 | TGATCGA | 981 | TCTCCTCCA | | M1- | 1146 | AGTGACA | 789 | CCTTGCCGT |
| | 179 | | GGC | | C | | 371 | | CCA | | A |
| | M1- | 955 | AACATCG | 980 | CTAATAGGC | | M1- | 1147 | TCGCGAT | 788 | AGACAGGA |
| | 180 | | TAG | | T | | 372 | | GTC | | AT |
| Group | M1- | 956 | GTTGCG | 979 | AGCCGTAA | Group | M1- | 1148 | CGAGTC | 787 | GTTGTGCA |
| 46 | 181 | | CGAA | | TA | 94 | 373 | | AGTC | | GC |
| | M1- | 957 | ACAAGTA | 978 | GTTGCCTG | | M1- | 1149 | TTGCCA | 786 | TCAAGAGC |
| | 182 | | AGC | | AC | | 374 | | GTGA | | AT |
| | M1- | 958 | CGCTTCT | 977 | CAGTAACT | | M1- | 1150 | AACAAG | 785 | CGCTACTGC |
| | 183 | | CCT | | CT | | 375 | | CACT | | A |
| | M1- | 959 | TAGCAAG | 976 | TCAATGGC | | M1- | 1151 | GCTTGTT | 784 | AAGCCTATT |
| | 184 | | TTG | | GG | | 376 | | CAG | | G |
| Group | M1- | 960 | AGGCCTC | 975 | ATCATCAGC | Group | M1- | 1152 | TAGTGAT | 783 | AATACGCC |
| 47 | 185 | | TTC | | C | 95 | 377 | | GTG | | TA |
| | M1- | 961 | GTAATGT | 974 | TCGTGATC | | M1- | 1153 | CGTGTG | 782 | GTCTAAGG |
| | 186 | | CGT | | AG | | 378 | | ACAT | | CT |
| | M1- | 962 | CACTGCA | 973 | GATGCGCAT | | M1- | 1154 | ATCCACC | 781 | CGAGGCTT |
| | 187 | | GAG | | A | | 379 | | ACC | | AG |
| | M1- | 963 | TCTGAAG | 972 | CGACATGTG | | M1- | 1155 | GCAACTG | 780 | TCGCTTAAG |
| | 188 | | ACA | | T | | 380 | | TGA | | C |
| Group | M1- | 964 | GCTAAGG | 971 | GTTGCAAGC | Group | M1- | 1156 | ATCCACC | 779 | CGGATGCA |
| 48 | 189 | | ATA | | C | 96 | 381 | | GGT | | AG |
| | M1- | 965 | CACGGTT | 970 | TCGATGCTA | | M1- | 1157 | CCGTCAT | 778 | GTCTCAATG |
| | 190 | | GGT | | A | | 382 | | TAC | | A |
| | M1- | 966 | TGACCAC | 969 | CAACGTGA | | M1- | 1158 | TGAGTGG | 777 | AATGGCGC |
| | 191 | | CAG | | GT | | 383 | | CTA | | TC |
| | M1- | 967 | ATGTTCAT | 968 | AGCTACTCT | | M1- | 1159 | GATAGTA | 776 | TCACATTGC |
| | 192 | | CC | | G | | 384 | | ACG | | T |

TABLE 2

| | | | | | | | | | | | Index |
|---|---|---|---|---|---|---|---|---|---|---|---|
| | | | Index | | Index | | | | Index | | corres- |
| | | | corres- | | corres- | | | | corres- | | ponding |
| | | | ponding | | ponding | | | | ponding | | to non- |
| Bal- | | | to phos- | | to non- | Bal- | | | to phos- | | phos- |
| anced | | SEQ | phoryl- | SEQ | phosphoryl- | anced | | SEQ | phoryl- | SEQ | phoryl- |
| combi- | Num- | ID | ation | ID | ation | combin- | Num- | ID | ation | ID | ation |
| nation | ber | NO: | end | NO: | end | ation | ber | NO: | end | NO: | end |
| Group | M2- | 1 | CCGTTGT | 193 | GCTCTGT | Group | M2- | 385 | ATAGGCG | 577 | TCAAGTCG |
| 1 | 001 | | GAG | | GTC | 49 | 193 | | TTA | | CC |
| | M2- | 2 | AATGGTG | 194 | TGGTGCG | | M2- | 386 | TGCCATA | 578 | GTGTACAC |
| | 002 | | AGT | | CAA | | 194 | | CGG | | TA |
| | M2- | 3 | TGCAACC | 195 | AACACAC | | M2- | 387 | CATTCGT | 579 | CGCGTAGT |
| | 003 | | TTC | | TGT | | 195 | | GCC | | GT |
| | M2- | 4 | GTACCAA | 196 | CTAGATA | | M2- | 388 | GCGATA | 580 | AATCCGTA |
| | 004 | | CCA | | ACG | | 196 | | CAAT | | AG |
| Group | M2- | 5 | TGCGTTC | 197 | AGGCCGT | Group | M2- | 389 | TTAACGC | 581 | TGCAGAAT |
| 2 | 005 | | TGC | | TAC | 50 | 197 | | GAC | | CC |
| | M2- | 6 | CTGTCGT | 198 | CTTGGAC | | M2- | 390 | CATGTT | 582 | ACTTAGGA |
| | 006 | | GAT | | CTG | | 198 | | GTCA | | GT |
| | M2- | 7 | ACAAGC | 199 | TCCAATA | | M2- | 391 | AGGCGA | 583 | CTGGTCTC |
| | 007 | | AATG | | GGA | | 199 | | TCTG | | AA |
| | M2- | 8 | GATCAAG | 200 | GAATTCG | | M2- | 392 | GCCTAC | 584 | GAACCTCG |
| | 008 | | CCA | | ACT | | 200 | | AAGT | | TG |
| Group | M2- | 9 | ACTTAGA | 201 | GAATTCGC | Group | M2- | 393 | GAGGTT | 585 | TGGCTACA |
| 3 | 009 | | TCG | | GA | 51 | 201 | | AGTA | | TA |
| | M2- | 10 | TTAGGAG | 202 | CGTGGTC | | M2- | 394 | AGTTCC | 586 | CCATGTAG |
| | 010 | | GAA | | ACT | | 202 | | GCCT | | AG |
| | M2- | 11 | CGCATCC | 203 | TTCACGA | | M2- | 395 | CCACAG | 587 | GTTGACGT |
| | 011 | | ATC | | TAC | | 203 | | CAAG | | CC |
| | M2- | 12 | GAGCCTT | 204 | ACGCAAT | | M2- | 396 | TTCAGAT | 588 | AACACGTC |
| | 012 | | CGT | | GTG | | 204 | | TGC | | GT |
| Group | M2- | 13 | ACTCGCG | 205 | ACAATCC | Group | M2- | 397 | CGGATA | 589 | TAACCACC |
| 4 | 013 | | GTA | | GGC | 52 | 205 | | CACT | | TC |
| | M2- | 14 | CACGAA | 206 | TTGTGGT | | M2- | 398 | GACGAT | 590 | ACGTGCTT |
| | 014 | | CACT | | ATG | | 206 | | AGGA | | AT |
| | M2- | 15 | GTAACTA | 207 | CGCCATGT | | M2- | 399 | TCTCGGT | 591 | GTCGTGGA |
| | 015 | | TGG | | AA | | 207 | | CTG | | GA |
| | M2- | 16 | TGGTTGT | 208 | GATGCAA | | M2- | 400 | ATATCCG | 592 | CGTAATAGC |
| | 016 | | CAC | | CCT | | 208 | | TAC | | G |
| Group | M2- | 17 | AGGTCA | 209 | TATCGTT | Group | M2- | 401 | AGATAGC | 593 | AACCGTGG |
| 5 | 017 | | CTAG | | GTG | 53 | 209 | | CTT | | TA |
| | M2- | 18 | CCTGATA | 210 | ATGACAG | | M2- | 402 | CTTACCA | 594 | GTTGTACTA |
| | 018 | | GCC | | CAC | | 210 | | TGC | | C |
| | M2- | 19 | GACCTG | 211 | CGCTTCA | | M2- | 403 | TCGGTTG | 595 | TCATCGAA |
| | 019 | | GATT | | TGT | | 211 | | GAG | | GG |
| | M2- | 20 | TTAAGCT | 212 | GCAGAGC | | M2- | 404 | GACCGAT | 596 | CGGAACTC |
| | 020 | | CGA | | ACA | | 212 | | ACA | | CT |
| Group | M2- | 21 | CGGTGAA | 213 | CTACGCG | Group | M2- | 405 | AACAACC | 597 | AGTACAAC |
| 6 | 021 | | GTC | | AAG | 54 | 213 | | TCA | | TG |
| | M2- | 22 | GTAACTG | 214 | AAGGTGA | | M2- | 406 | CGGCTGT | 598 | CAAGTCTG |
| | 022 | | CAT | | TTC | | 214 | | AAC | | CA |
| | M2- | 23 | TCTCTGT | 215 | GCTTATTG | | M2- | 407 | GTAGCAG | 599 | GTGCAGCT |
| | 023 | | TGA | | CT | | 215 | | GTG | | AC |
| | M2- | 24 | AACGACC | 216 | TGCACAC | | M2- | 408 | TCTTGTA | 600 | TCCTGTGA |
| | 024 | | ACG | | CGA | | 216 | | CGT | | GT |
| Group | M2- | 25 | GCGAATC | 217 | ACTGGATC | Group | M2- | 409 | ACGTGA | 601 | CACTAATC |
| 7 | 025 | | AGC | | CG | 55 | 217 | | ACAC | | AG |
| | M2- | 26 | AACTTCG | 218 | GAGAACA | | M2- | 410 | CGCACT | 602 | ACTAGCGG |
| | 026 | | GTA | | GAT | | 218 | | GGTA | | CT |
| | M2- | 27 | TGTGGA | 219 | TTACCTC | | M2- | 411 | GTAGAG | 603 | GTAGTTCA |
| | 027 | | ATAG | | AGA | | 219 | | CTCT | | TC |
| | M2- | 28 | CTACCGT | 220 | CGCTTGG | | M2- | 412 | TATCTCT | 604 | TGGCCGAT |
| | 028 | | CCT | | TTC | | 220 | | AGG | | GA |
| Group | M2- | 29 | CCATAAG | 221 | TCACCTCT | Group | M2- | 413 | AACAACT | 605 | ACCATCCG |
| 8 | 029 | | AGG | | CG | 56 | 221 | | TGG | | AA |
| | M2- | 30 | GTCGGTA | 222 | ATCAGCG | | M2- | 414 | CCACCAC | 606 | CATTCGATT |
| | 030 | | GTC | | CTC | | 222 | | CTA | | C |
| | M2- | 31 | TGTCTGC | 223 | CGGTAATG | | M2- | 415 | GTGGTGA | 607 | GTAGGATAG |
| | 031 | | CAT | | AA | | 223 | | GAC | | G |
| | M2- | 32 | AAGACCT | 224 | GATGTGA | | M2- | 416 | TGTTGTG | 608 | TGGCATGCC |
| | 032 | | TCA | | AGT | | 224 | | ACT | | T |
| Group | M2- | 33 | CCGATTC | 225 | TAGTTGG | Group | M2- | 417 | CCATTG | 609 | CGTAGATC |
| 9 | 033 | | GAT | | TAC | 57 | 225 | | AATG | | GC |
| | M2- | 34 | ATTGCAG | 226 | CGAGACA | | M2- | 418 | TTCAATC | 610 | TAAGCGCG |
| | 034 | | CCA | | ACT | | 226 | | GAC | | TG |
| | M2- | 35 | GAATACT | 227 | GTTCCAC | | M2- | 419 | AGGCGC | 611 | GTGCTTGT |
| | 035 | | TGC | | CGA | | 227 | | TTCT | | AT |
| | M2- | 36 | TGCCGG | 228 | ACCAGTT | | M2- | 420 | GATGCA | 612 | ACCTACAA |
| | 036 | | AATG | | GTG | | 228 | | GCGA | | CA |
| Group | M2- | 37 | ATGCGG | 229 | CAAGCAG | Group | M2- | 421 | CACGAT | 613 | AGTCATGA |
| 10 | 037 | | ATCC | | CAG | 58 | 229 | | GTGA | | CA |
| | M2- | 38 | CCTTCAT | 230 | GCGAACT | | M2- | 422 | TGGTCC | 614 | TTGAGCCT |
| | 038 | | GAA | | GGT | | 230 | | AATT | | GG |
| | M2- | 39 | GACGATC | 231 | TGCTGTC | | M2- | 423 | ATTAGGT | 615 | CAAGTATCT |
| | 039 | | ATG | | TTA | | 231 | | GCC | | C |
| | M2- | 40 | TGAATCG | 232 | ATTCTGA | | M2- | 424 | GCACTAC | 616 | GCCTCGAG |
| | 040 | | CGT | | ACC | | 232 | | CAG | | AT |
| Group | M2- | 41 | TACACTC | 233 | ACCAATC | Group | M2- | 425 | AAGTGA | 617 | CACTCGTA |
| 11 | 041 | | GGT | | GTT | 59 | 233 | | TCCA | | GC |
| | M2- | 42 | ATACAGG | 234 | TGTTCAG | | M2- | 426 | GTACATC | 618 | GCTGGTAC |
| | 042 | | ACG | | CAA | | 234 | | TTC | | CT |
| | M2- | 43 | CGTGTCT | 235 | CAGGTGT | | M2- | 427 | TCTGCC | 619 | AGGATCCT |
| | 043 | | CAC | | AGC | | 235 | | GGAT | | AA |
| | M2- | 44 | GCGTGAA | 236 | GTACGCAT | | M2- | 428 | CGCATGA | 620 | TTACAAGG |
| | 044 | | TTA | | CG | | 236 | | AGG | | TG |
| Group | M2- | 45 | TTCGTTC | 237 | CAACACC | Group | M2- | 429 | ACTGAG | 621 | AATGAGGC |
| 12 | 045 | | ACA | | GAG | 60 | 237 | | TAAG | | TA |
| | M2- | 46 | GAGACG | 238 | GTTGCAG | | M2- | 430 | CTCACAG | 622 | GTAAGTCG |
| | 046 | | GTTG | | ATC | | 238 | | CGA | | CG |
| | M2- | 47 | ACACAAT | 239 | TGGTGGT | | M2- | 431 | GAGTTC | 623 | TGCTCCTA |
| | 047 | | GGT | | TGT | | 239 | | AGCT | | AC |
| | M2- | 48 | CGTTGCA | 240 | ACCATTA | | M2- | 432 | TGACGT | 624 | CCGCTAAT |
| | 048 | | CAC | | CCA | | 240 | | CTTC | | GT |
| Group | M2- | 49 | GAACACT | 241 | AGTTGTCT | Group | M2- | 433 | CGCCTGT | 625 | AGTTAGTC |
| 13 | 049 | | ATC | | GC | 61 | 241 | | TGT | | AC |
| | M2- | 50 | TCTGCGG | 242 | CTAGAGG | | M2- | 434 | GAGAATA | 626 | CTCGTTGTC |
| | 050 | | TAA | | ACA | | 242 | | GCA | | G |
| | M2- | 51 | AGGTTAA | 243 | GCGACCT | | M2- | 435 | TTAGCAC | 627 | GCGAGCCA |
| | 051 | | GCG | | CAT | | 243 | | ATC | | TA |
| | M2- | 52 | CTCAGTC | 244 | TACCTAAG | | M2- | 436 | ACTTGCG | 628 | TAACCAAG |
| | 052 | | CGT | | TG | | 244 | | CAG | | GT |
| Group | M2- | 53 | CGCAATG | 245 | GTGTTATC | Group | M2- | 437 | CGAGAA | 629 | CCAGATGT |
| 14 | 053 | | AAC | | TC | 62 | 245 | | TTCA | | GC |
| | M2- | 54 | TTGGCAC | 246 | TCAGACG | | M2- | 438 | GCGTTC | 630 | GATTCGTG |
| | 054 | | TTA | | TCG | | 246 | | GCTT | | AG |
| | M2- | 55 | GCATTCA | 247 | AACCGGA | | M2- | 439 | TTCCGTA | 631 | TTGCTCAC |
| | 055 | | GCG | | AGT | | 247 | | AGC | | CA |
| | M2- | 56 | AATCGGT | 248 | CGTACTCG | | M2- | 440 | AATACGC | 632 | AGCAGACA |
| | 056 | | CGT | | AA | | 248 | | GAG | | TT |
| Group | M2- | 57 | ATATGGC | 249 | ACTCGAA | Group | M2- | 441 | CGTGATT | 633 | TAGCGGAG |
| 15 | 057 | | AGA | | GGT | 63 | 249 | | GGT | | TG |
| | M2- | 58 | CGGACT | 250 | CGCTTGT | | M2- | 442 | ATCATGC | 634 | AGCAACTA |
| | 058 | | GTCT | | AAG | | 250 | | TAC | | GA |
| | M2- | 59 | GATCAAT | 251 | GTAACCG | | M2- | 443 | GCATCCG | 635 | CTAGTTCTC |
| | 059 | | CTG | | CTC | | 251 | | ATG | | T |
| | M2- | 60 | TCCGTCA | 252 | TAGGATCT | | M2- | 444 | TAGCGAA | 636 | GCTTCAGC |
| | 060 | | GAC | | CA | | 252 | | CCA | | AC |
| Group | M2- | 61 | AGAGTGC | 253 | GTCCGATT | Group | M2- | 445 | GATCCT | 637 | CATAATCC |
| 16 | 061 | | ATG | | AC | 64 | 253 | | CTCG | | GG |
| | M2- | 62 | GTTCGCA | 254 | CAGTCGG | | M2- | 446 | TTGAAG | 638 | GTCCGCTG |
| | 062 | | TAC | | ATT | | 254 | | GAAC | | AT |
| | M2- | 63 | TCGAATT | 255 | TGTGATCG | | M2- | 447 | CCATTCT | 639 | AGATCAGT |
| | 063 | | CGT | | CG | | 255 | | GTA | | CC |
| | M2- | 64 | CACTCAG | 256 | ACAATCA | | M2- | 448 | AGCGGA | 640 | TCGGTGAA |
| | 064 | | GCA | | CGA | | 256 | | ACGT | | TA |
| Group | M2- | 65 | CCTTGGT | 257 | TGTGACTA | Group | M2- | 449 | TTCCACC | 641 | CAAGCCTG |
| 17 | 065 | | GCA | | CA | 65 | 257 | | TGG | | GT |
| | M2- | 66 | GAGCATC | 258 | ACACTTG | | M2- | 450 | GCATCTT | 642 | GTCCTTGCC |
| | 066 | | CAT | | CGG | | 258 | | CTT | | A |
| | M2- | 67 | AGAGTCG | 259 | CTGTGGCT | | M2- | 451 | AATGGAG | 643 | TCTAGGATT |
| | 067 | | ATC | | AT | | 259 | | AAC | | G |
| | M2- | 68 | TTCACAA | 260 | GACACAA | | M2- | 452 | CGGATGA | 644 | AGGTAACA |
| | 068 | | TGG | | GTC | | 260 | | GCA | | AC |
| Group | M2- | 69 | GTATCCT | 261 | TCATACTG | Group | M2- | 453 | CATGCC | 645 | CTGGAGGT |
| 18 | 069 | | CAG | | TC | 66 | 261 | | TATG | | CT |
| | M2- | 70 | TACCGAG | 262 | GATACGG | | M2- | 454 | GCGTGGA | 646 | AGCTCTAC |
| | 070 | | TGT | | AGG | | 262 | | CAA | | AA |
| | M2- | 71 | ACTGTGC | 263 | CGGCTAA | | M2- | 455 | AGCAATG | 647 | GATATATGG |
| | 071 | | ACC | | CCA | | 263 | | GCT | | C |
| | M2- | 72 | CGGAATA | 264 | ATCGGTC | | M2- | 456 | TTACTAC | 648 | TCACGCCA |
| | 072 | | GTA | | TAT | | 264 | | TGC | | TG |
| Group | M2- | 73 | GTGTCTA | 265 | GCCTCCTG | Group | M2- | 457 | CGGTAAT | 649 | TGGATCGG |
| 19 | 073 | | CAG | | TT | 67 | 265 | | ATC | | TC |
| | M2- | 74 | TACGTATA | 266 | TATAGGAC | | M2- | 458 | ACAGCC | 650 | ATATAGCC |
| | 074 | | CC | | AC | | 266 | | GGAA | | GG |
| | M2- | 75 | AGTAAGC | 267 | AGAGAAC | | M2- | 459 | GTTCTG | 651 | CCTCCATT |
| | 075 | | GGT | | TGA | | 267 | | CTCG | | AT |
| | M2- | 76 | CCACGCG | 268 | CTGCTTGA | | M2- | 460 | TACAGTA | 652 | GACGGTAA |
| | 076 | | TTA | | CG | | 268 | | CGT | | CA |
| Group | M2- | 77 | AGCTTAT | 269 | CCAACTAC | Group | M2- | 461 | CGTGGA | 653 | CAATTCCT |
| 20 | 077 | | GGC | | CA | 68 | 269 | | GTTG | | CA |
| | M2- | 78 | CCGAATG | 270 | GTGCGGC | | M2- | 462 | GTCTTCT | 654 | GCGAGAAG |
| | 078 | | ATT | | ATT | | 270 | | CGA | | AG |
| | M2- | 79 | GTTGGC | 271 | TGTGAAG | | M2- | 463 | TCACCT | 655 | TTCCAGTA |
| | 079 | | ACCA | | GAG | | 271 | | CAAT | | GT |
| | M2- | 80 | TAACCGC | 272 | AACTTCT | | M2- | 464 | AAGAAG | 656 | AGTGCTGC |
| | 080 | | TAG | | TGC | | 272 | | AGCC | | TC |
| Group | M2- | 81 | AGAAGTA | 273 | TCGTGGA | Group | M2- | 465 | AACGCCG | 657 | TACCGCAC |
| 21 | 081 | | GGA | | ATT | 69 | 273 | | TCA | | AT |
| | M2- | 82 | CTTCCAG | 274 | AATACAG | | M2- | 466 | GCGCTGA | 658 | GCAGTACA |
| | 082 | | TTG | | CGC | | 274 | | ATC | | CG |
| | M2- | 83 | GAGGTG | 275 | GTCCTCC | | M2- | 467 | CTAAGTT | 659 | CGTACGGTT |
| | 083 | | CAAC | | TAA | | 275 | | GGT | | A |
| | M2- | 84 | TCCTACT | 276 | CGAGATT | | M2- | 468 | TGTTAAC | 660 | ATGTATTGG |
| | 084 | | CCT | | GCG | | 276 | | CAG | | C |
| Group | M2- | 85 | TCCGACA | 277 | CCGTAGT | Group | M2- | 469 | CGATACC | 661 | CTGCCTTA |
| 22 | 085 | | GTT | | TGG | 70 | 277 | | TAG | | GT |
| | M2- | 86 | AGTAGTT | 278 | TTAGCTC | | M2- | 470 | GCTATGA | 662 | GATGTGGT |
| | 086 | | ACC | | GTT | | 278 | | CCT | | AC |
| | M2- | 87 | GAATCG | 279 | GATCGCA | | M2- | 471 | TTCGCT | 663 | TCCTACAG |
| | 087 | | GTGG | | CAA | | 279 | | GGTC | | CG |
| | M2- | 88 | CTGCTAC | 280 | AGCATAG | | M2- | 472 | AAGCGA | 664 | AGAAGACC |
| | 088 | | CAA | | ACC | | 280 | | TAGA | | TA |
| Group | M2- | 89 | CGTTGAA | 281 | AGTCGCC | Group | M2- | 473 | TCTGAT | 665 | ACTGGTGC |
| 23 | 089 | | TGG | | ATT | 71 | 281 | | GGAA | | AA |
| | M2- | 90 | ATGGCTT | 282 | GAGTAAT | | M2- | 474 | AGAACG | 666 | CGCCAGAA |
| | 090 | | CTA | | TGG | | 282 | | TCCG | | TC |
| | M2- | 91 | GACATGC | 283 | CTAGCTA | | M2- | 475 | GTCTTC | 667 | GTAATACT |
| | 091 | | ACC | | GCA | | 283 | | CATT | | CG |
| | M2- | 92 | TCACACG | 284 | TCCATGG | | M2- | 476 | CAGCGA | 668 | TAGTCCTG |
| | 092 | | GAT | | CAC | | 284 | | ATGC | | GT |
| Group | M2- | 93 | CCAATAT | 285 | TAGCTTC | Group | M2- | 477 | CGAGCAC | 669 | AGTCTAAG |
| 24 | 093 | | GGT | | CGG | 72 | 285 | | TGT | | GA |
| | M2- | 94 | GTCTCGC | 286 | ACTTACAG | | M2- | 478 | GCTATTG | 670 | GAGGACGA |
| | 094 | | TCA | | CT | | 286 | | ATC | | TC |
| | M2- | 95 | TGGCAC | 287 | CGCAGAT | | M2- | 479 | TAGTACA | 671 | CCATGTTCA |
| | 095 | | GAAC | | ATA | | 287 | | CAG | | T |
| | M2- | 96 | AATGGTA | 288 | GTAGCGG | | M2- | 480 | ATCCGGT | 672 | TTCACGCTC |
| | 096 | | CTG | | TAC | | 288 | | GCA | | G |
| Group | M2- | 97 | GAGGCT | 289 | TCTAATC | Group | M2- | 481 | AACTTG | 673 | GATCTAGC |
| 25 | 097 | | GAAT | | GCA | 73 | 289 | | GTGT | | AC |
| | M2- | 98 | AGTAAGA | 290 | AAGCTAA | | M2- | 482 | GCACGA | 674 | CTGTCGATG |
| | 098 | | GTC | | CAC | | 290 | | AGTG | | G |
| | M2- | 99 | TCCTGAC | 291 | CGAGCGT | | M2- | 483 | CGTAAC | 675 | TCCAGTTA |
| | 099 | | TGA | | AGT | | 291 | | TACC | | CA |
| | M2- | 100 | CTACTCT | 292 | GTCTGCG | | M2- | 484 | TTGGCT | 676 | AGAGACCG |
| | 100 | | CCG | | TTG | | 292 | | CCAA | | TT |
| Group | M2- | 101 | AGGTCTG | 293 | TGAACAA | Group | M2- | 485 | GTTCATC | 677 | GTACAATTC |
| 26 | 101 | | ATT | | CTC | 74 | 293 | | GAG | | G |
| | M2- | 102 | CTTGAGC | 294 | ACTCAGT | | M2- | 486 | TACACAT | 678 | CATTCCGC |
| | 102 | | CAG | | GCT | | 294 | | CGC | | AA |
| | M2- | 103 | GAACGAA | 295 | CTCGTTCA | | M2- | 487 | ACGTGCG | 679 | TGGATGCA |
| | 103 | | GGC | | GG | | 295 | | ACT | | GC |
| | M2- | 104 | TCCATCT | 296 | GAGTGCG | | M2- | 488 | CGAGTG | 680 | ACCGGTAG |
| | 104 | | TCA | | TAA | | 296 | | ATTA | | TT |
| Group | M2- | 105 | CGTCACG | 297 | CGCGACC | Group | M2- | 489 | CAGCTC | 681 | ATGCGTTC |
| 27 | 105 | | CAA | | ATT | 75 | 297 | | CTCT | | CG |
| | M2- | 106 | GCCTGTT | 298 | AATACTGG | | M2- | 490 | GCAGATA | 682 | CCAACACT |
| | 106 | | ATT | | CA | | 298 | | AGC | | AC |
| | M2- | 107 | ATAACAA | 299 | TTACGGA | | M2- | 491 | TGTTGAT | 683 | GACGTCGA |
| | 107 | | GCG | | CGC | | 299 | | GAG | | TA |
| | M2- | 108 | TAGGTGC | 300 | GCGTTAT | | M2- | 492 | ATCACGG | 684 | TGTTAGAG |
| | 108 | | TGC | | TAG | | 300 | | CTA | | GT |
| Group | M2- | 109 | GATCTGA | 301 | TCAGTTC | Group | M2- | 493 | TTCAGA | 685 | CCTCTCAC |
| 28 | 109 | | ACA | | GAT | 76 | 301 | | ACAG | | GT |
| | M2- | 110 | CTCAATG | 302 | CTGAGCA | | M2- | 494 | CAAGCC | 686 | GTCTCAGT |
| | 110 | | TGT | | AGC | | 302 | | GTGA | | CG |
| | M2- | 111 | TCGTGAC | 303 | GACTAAG | | M2- | 495 | AGGTTG | 687 | TAGGAGCA |
| | 111 | | GAC | | TTG | | 303 | | TGTT | | TA |
| | M2- | 112 | AGAGCC | 304 | AGTCCGT | | M2- | 496 | GCTCATC | 688 | AGAAGTTG |
| | 112 | | TCTG | | CCA | | 304 | | ACC | | AC |
| Group | M2- | 113 | CGAAGTA | 305 | GCGGATA | Group | M2- | 497 | CCATACT | 689 | TGGTTCGT |
| 29 | 113 | | CCG | | TTC | 77 | 305 | | CAG | | CG |
| | M2- | 114 | ACCGAAT | 306 | ATACCGC | | M2- | 498 | GTGAGTA | 690 | ACCGCACG |
| | 114 | | AGA | | AAG | | 306 | | TTC | | TT |
| | M2- | 115 | TAGTCGC | 307 | TGTTGCG | | M2- | 499 | TGTGCG | 691 | CTTAAGTC |
| | 115 | | TAT | | CGT | | 307 | | GACA | | AC |
| | M2- | 116 | GTTCTCG | 308 | CACATAT | | M2- | 500 | AACCTA | 692 | GAACGTAA |
| | 116 | | GTC | | GCA | | 308 | | CGGT | | GA |
| Group | M2- | 117 | AACACG | 309 | AGCGTGA | Group | M2- | 501 | TGAACC | 693 | CTCCGCAA |
| 30 | 117 | | AGAA | | CCT | 78 | 309 | | GAAG | | GT |
| | M2- | 118 | CTTCGCT | 310 | GTATAACG | | M2- | 502 | ACCTGG | 694 | GATGTTGC |
| | 118 | | CTC | | AC | | 310 | | CGTA | | AA |
| | M2- | 119 | GCGTATC | 311 | TAGAGCTA | | M2- | 503 | CAGCTAT | 695 | TGATAATGC |
| | 119 | | TCG | | TG | | 311 | | CCT | | G |
| | M2- | 120 | TGAGTAG | 312 | CCTCCTG | | M2- | 504 | GTTGATA | 696 | ACGACGCT |
| | 120 | | AGT | | TGA | | 312 | | TGC | | TC |
| Group | M2- | 121 | TGGAGTT | 313 | CTCAAGG | Group | M2- | 505 | GCCTCAT | 697 | GCACAGAG |
| 31 | 121 | | GCG | | CTC | 79 | 313 | | GAC | | AC |
| | M2- | 122 | GTTGTAA | 314 | GCACCTT | | M2- | 506 | ATACACA | 698 | CTTGCACT |
| | 122 | | CGT | | AGG | | 314 | | CCT | | CA |
| | M2- | 123 | ACCTACC | 315 | TGTGTAC | | M2- | 507 | TATGGTG | 699 | TACTTCGA |
| | 123 | | TAA | | TCT | | 315 | | ATG | | GG |
| | M2- | 124 | CAACCGG | 316 | AAGTGCA | | M2- | 508 | CGGATG | 700 | AGGAGTTC |
| | 124 | | ATC | | GAA | | 316 | | CTGA | | TT |
| Group | M2- | 125 | GATAGCT | 317 | TGCGACG | Group | M2- | 509 | CCGCTTG | 701 | CGGACGTT |
| 32 | 125 | | TCG | | ATA | 80 | 317 | | TTG | | AA |
| | M2- | 126 | CCGCAA | 318 | ATGACGA | | M2- | 510 | TTCTGCT | 702 | ACTCAACG |
| | 126 | | CGAT | | TCG | | 318 | | ACA | | GC |
| | M2- | 127 | AGATCG | 319 | CATTGTT | | M2- | 511 | AGAACA | 703 | GTATGCAAT |
| | 127 | | GATA | | CAC | | 319 | | AGGT | | G |
| | M2- | 128 | TTCGTTA | 320 | GCACTAC | | M2- | 512 | GATGAGC | 704 | TACGTTGCC |
| | 128 | | CGC | | GGT | | 320 | | CAC | | T |
| Group | M2- | 129 | AAGAGA | 321 | GCAACCT | Group | M2- | 513 | ACAAGC | 705 | ACTGGCTA |
| 33 | 129 | | CACA | | GAG | 81 | 321 | | TCTA | | AC |
| | M2- | 130 | CCACTTG | 322 | TAGTAGC | | M2- | 514 | CTGCAG | 706 | CTACTGAT |
| | 130 | | GAT | | ACT | | 322 | | GAAG | | GT |
| | M2- | 131 | GTCTCCT | 323 | AGCCTTA | | M2- | 515 | TGTTCTA | 707 | GACTATGG |
| | 131 | | TGC | | TTC | | 323 | | GCT | | CA |
| | M2- | 132 | TGTGAG | 324 | CTTGGAG | | M2- | 516 | GACGTA | 708 | TGGACACC |
| | 132 | | ACTG | | CGA | | 324 | | CTGC | | TG |
| Group | M2- | 133 | AGGATAG | 325 | AGACGTG | Group | M2- | 517 | GTAAGG | 709 | TCTCACCA |
| 34 | 133 | | CCG | | TTA | 82 | 325 | | TGTT | | GG |
| | M2- | 134 | TCTGCTA | 326 | CCGGTGA | | M2- | 518 | AAGGCT | 710 | AACGTAGG |
| | 134 | | TAC | | GAT | | 326 | | AAGC | | CT |
| | M2- | 135 | GTCTACT | 327 | GTCTCAC | | M2- | 519 | CGCTTC | 711 | CGATCTTC |
| | 135 | | GGT | | AGC | | 327 | | CTAA | | TC |
| | M2- | 136 | CAACGG | 328 | TATAACT | | M2- | 520 | TCTCAA | 712 | GTGAGGAT |
| | 136 | | CATA | | CCG | | 328 | | GCCG | | AA |
| Group | M2- | 137 | GTACAGC | 329 | GAAGTCC | Group | M2- | 521 | GCTCTAT | 713 | GCACTTCAT |
| 35 | 137 | | GGA | | TTG | 83 | 329 | | CCG | | G |
| | M2- | 138 | CGCATAG | 330 | TCCAATGC | | M2- | 522 | TGAGCTA | 714 | CTGGACAG |
| | 138 | | CAC | | CA | | 330 | | GTA | | GT |
| | M2- | 139 | ACTGGTT | 331 | ATGTCGA | | M2- | 523 | CTGTAGG | 715 | TGTTGGTCC |
| | 139 | | ATG | | GAC | | 331 | | AGC | | A |
| | M2- | 140 | TAGTCCA | 332 | CGTCGATA | | M2- | 524 | AACAGCC | 716 | AACACAGT |
| | 140 | | TCT | | GT | | 332 | | TAT | | AC |
| Group | M2- | 141 | TGACGG | 333 | GATTACA | Group | M2- | 525 | GCGCATC | 717 | TTCAAGGA |
| 36 | 141 | | TTGC | | GGT | 84 | 333 | | GTT | | GA |
| | M2- | 142 | GCTAACC | 334 | TCCGGAG | | M2- | 526 | TTAAGC | 718 | CCGTCTAG |
| | 142 | | GTG | | AAC | | 334 | | ATCC | | TG |
| | M2- | 143 | ATGGTTG | 335 | ATACCGT | | M2- | 527 | CGCGTAT | 719 | AGAGGATC |
| | 143 | | ACT | | CTA | | 335 | | CAG | | CT |
| | M2- | 144 | CACTCAA | 336 | CGGATTCT | | M2- | 528 | AATTCG | 720 | GATCTCCT |
| | 144 | | CAA | | CG | | 336 | | GAGA | | AC |
| Group | M2- | 145 | TAAGCTA | 337 | AACTGAC | Group | M2- | 529 | AGCGAA | 721 | ACCATGAG |
| 37 | 145 | | CCA | | CGT | 85 | 337 | | TGAT | | AC |
| | M2- | 146 | AGCTGAG | 338 | GTACTTAT | | M2- | 530 | CATCGT | 722 | TGTTCATC |
| | 146 | | GTG | | CC | | 338 | | CCTG | | TG |
| | M2- | 147 | CTTCTCC | 339 | CGTACGTA | | M2- | 531 | GCGTTCA | 723 | CTAGACCT |
| | 147 | | AGC | | AG | | 339 | | AGA | | GT |
| | M2- | 148 | GCGAAGT | 340 | TCGGACG | | M2- | 532 | TTAACGG | 724 | GAGCGTGA |
| | 148 | | TAT | | GTA | | 340 | | TCC | | CA |
| Group | M2- | 149 | ATCCATG | 341 | AGGACAG | Group | M2- | 533 | CGGATAT | 725 | TGCTGGAT |
| 38 | 149 | | TCC | | AGG | 86 | 341 | | CAA | | CT |
| | M2- | 150 | TAGGTCT | 342 | TCTGACA | | M2- | 534 | GTACCG | 726 | AAGGCACA |
| | 150 | | CGG | | CAA | | 342 | | AAGC | | AC |
| | M2- | 151 | GCATGAA | 343 | GAATGTT | | M2- | 535 | TACTACC | 727 | CTAATCTGG |
| | 151 | | GAA | | GTC | | 343 | | GTT | | A |
| | M2- | 152 | CGTACGC | 344 | CTCCTGC | | M2- | 536 | ACTGGTG | 728 | GCTCATGCT |
| | 152 | | ATT | | TCT | | 344 | | TCG | | G |
| Group | M2- | 153 | GTGAATT | 345 | AGAAGAT | Group | M2- | 537 | CGTAATT | 729 | GATCCTCAA |
| 39 | 153 | | GAC | | GCA | 87 | 345 | | CAG | | C |
| | M2- | 154 | TACCTAG | 346 | GTTCCGCT | | M2- | 538 | GAAGCG | 730 | TGCAGCAG |
| | 154 | | ACT | | TC | | 346 | | GTCA | | CA |
| | M2- | 155 | AGATGCC | 347 | CACTATGA | | M2- | 539 | TTCCGAA | 731 | CTAGTGTTG |
| | 155 | | TGG | | GT | | 347 | | GGT | | G |
| | M2- | 156 | CCTGCGA | 348 | TCGGTCA | | M2- | 540 | ACGTTCC | 732 | ACGTAAGC |
| | 156 | | CTA | | CAG | | 348 | | ATC | | TT |
| Group | M2- | 157 | CCGGTCA | 349 | GTCAGGC | Group | M2- | 541 | TAGGAC | 733 | AGTGGATC |
| 40 | 157 | | ACT | | ATG | 88 | 349 | | CGTC | | AA |
| | M2- | 158 | GTAAGAT | 350 | TCAGTCA | | M2- | 542 | AGTCCT | 734 | TACATCAA |
| | 158 | | GAG | | TGT | | 350 | | GTAA | | CG |
| | M2- | 159 | TACTCGC | 351 | AAGCAAT | | M2- | 543 | CTAAGG | 735 | CTACCGCG |
| | 159 | | CGA | | GAC | | 351 | | AAGG | | TT |
| | M2- | 160 | AGTCATG | 352 | CGTTCTG | | M2- | 544 | GCCTTAT | 736 | GCGTATGT |
| | 160 | | TTC | | CCA | | 352 | | CCT | | GC |
| Group | M2- | 161 | TCGAGGT | 353 | GAAGACG | Group | M2- | 545 | CTTGGTG | 737 | ACACTAGC |
| 41 | 161 | | TCC | | AGT | 89 | 353 | | ACC | | CT |
| | M2- | 162 | GATGCTA | 354 | CTTCTTC | | M2- | 546 | AGAACA | 738 | TGTGAGCG |
| | 162 | | GAA | | CTC | | 354 | | CTTG | | AA |
| | M2- | 163 | ATCTAAC | 355 | AGGAGAA | | M2- | 547 | GCGCACA | 739 | CACTCCATG |
| | 163 | | CTG | | GAA | | 355 | | CAA | | C |
| | M2- | 164 | CGACTC | 356 | TCCTCGT | | M2- | 548 | TACTTGT | 740 | GTGAGTTA |
| | 164 | | GAGT | | TCG | | 356 | | GGT | | TG |
| Group | M2- | 165 | ACCATAC | 357 | ACATGCG | Group | M2- | 549 | TTCAACA | 741 | TACTCGGA |
| 42 | 165 | | TCC | | TAA | 90 | 357 | | AGC | | AG |
| | M2- | 166 | CGGTGTA | 358 | TGCAAGT | | M2- | 550 | CATGCAC | 742 | ATTGGCCTG |
| | 166 | | CAA | | AGC | | 358 | | GCT | | C |
| | M2- | 167 | GTTGCG | 359 | CTTCCAA | | M2- | 551 | GCATGTG | 743 | CGGCATTGT |
| | 167 | | GAGT | | GCT | | 359 | | TTA | | T |
| | M2- | 168 | TAACACT | 360 | GAGGTTC | | M2- | 552 | AGGCTGT | 744 | GCAATAAC |
| | 168 | | GTG | | CTG | | 360 | | CAG | | CA |
| Group | M2- | 169 | CAAGTAA | 361 | CACGTTG | Group | M2- | 553 | TTGTAC | 745 | GTAGCGAG |
| 43 | 169 | | CGG | | CAG | 91 | 361 | | GGCT | | TT |
| | M2- | 170 | TCGAGT | 362 | GTGAACA | | M2- | 554 | CGAGTT | 746 | AACCGTTC |
| | 170 | | GTAT | | AGA | | 362 | | AAGG | | GA |
| | M2- | 171 | GTTCCGT | 363 | TGTTGGT | | M2- | 555 | GATCCAC | 747 | TCTATCGTA |
| | 171 | | ACC | | GTC | | 363 | | TTA | | C |
| | M2- | 172 | AGCTACC | 364 | ACACCAC | | M2- | 556 | ACCAGGT | 748 | CGGTAACA |
| | 172 | | GTA | | TCT | | 364 | | CAC | | CG |
| Group | M2- | 173 | TGATACG | 365 | AGACTCG | Group | M2- | 557 | TCCACG | 749 | GCAACTTG |
| 44 | 173 | | CGG | | CGA | 92 | 365 | | GTTC | | TC |
| | M2- | 174 | CCGACA | 366 | TAGTGAA | | M2- | 558 | ATATTCT | 750 | ATGGAGAC |
| | 174 | | CATC | | TCG | | 366 | | CGG | | GG |
| | M2- | 175 | GTTGGTT | 367 | CCTGCTT | | M2- | 559 | CGGCGA | 751 | CGTCTAGT |
| | 175 | | GAA | | AAT | | 367 | | AGAT | | AA |
| | M2- | 176 | AACCTGA | 368 | GTCAAGC | | M2- | 560 | GATGATC | 752 | TACTGCCA |
| | 176 | | TCT | | GTC | | 368 | | ACA | | CT |
| Group | M2- | 177 | ATTGAGC | 369 | CCTATAAT | Group | M2- | 561 | GACCTAC | 753 | GATCAACA |
| 45 | 177 | | ACT | | GC | 93 | 369 | | TAT | | AG |
| | M2- | 178 | TGAATTG | 370 | AGCTACC | | M2- | 562 | TGGAATA | 754 | ACATTCTTC |
| | 178 | | TGC | | AAT | | 370 | | CCA | | C |
| | M2- | 179 | GACCGAT | 371 | TTACGTG | | M2- | 563 | ATTGCCG | 755 | TTGGCTGC |
| | 179 | | GTG | | CCG | | 371 | | AGC | | GT |
| | M2- | 180 | CCGTCCA | 372 | GAGGCGT | | M2- | 564 | CCATGGT | 756 | CGCAGGAG |
| | 180 | | CAA | | GTA | | 372 | | GTG | | TA |
| Group | M2- | 181 | TAAGACG | 373 | GTTGCCAT | Group | M2- | 565 | AGATTGT | 757 | CGTGTATGT |
| 46 | 181 | | CCA | | TA | 94 | 373 | | GCG | | C |
| | M2- | 182 | CTCTCTC | 374 | ACCAATT | | M2- | 566 | GATGAAG | 758 | GTCTGTCA |
| | 182 | | TAG | | CGG | | 374 | | TGC | | AG |
| | M2- | 183 | GCTAGAT | 375 | CGATGAG | | M2- | 567 | TCGACTA | 759 | TCAACCGC |
| | 183 | | AGT | | ACT | | 375 | | CAT | | CA |
| | M2- | 184 | AGGCTG | 376 | TAGCTGC | | M2- | 568 | CTCCGCC | 760 | AAGCAGAT |
| | 184 | | AGTC | | GAC | | 376 | | ATA | | GT |
| Group | M2- | 185 | TCTAACG | 377 | CGAGTTG | Group | M2- | 569 | AGTTGCC | 761 | GACAAGTG |
| 47 | 185 | | TGT | | AAT | 95 | 377 | | GGA | | GT |
| | M2- | 186 | AGGCCT | 378 | GACACCT | | M2- | 570 | CCAACTT | 762 | CTGGTACA |
| | 186 | | CCAA | | TCC | | 378 | | AAC | | CA |
| | M2- | 187 | GAATTGA | 379 | TTGCAAC | | M2- | 571 | TAGGTGA | 763 | TGACCTGCT |
| | 187 | | ACC | | GTA | | 379 | | TCT | | G |
| | M2- | 188 | CTCGGAT | 380 | ACTTGGA | | M2- | 572 | GTCCAAG | 764 | ACTTGCATA |
| | 188 | | GTG | | CGG | | 380 | | CTG | | C |
| Group | M2- | 189 | GTTCTAA | 381 | GTCATCG | Group | M2- | 573 | TCCAAGC | 765 | AGTGAGGT |
| 48 | 189 | | CTC | | CGT | 96 | 381 | | TCC | | CA |
| | M2- | 190 | CCAACTC | 382 | TAGGAAT | | M2- | 574 | AGGTTCG | 766 | CTACTAAGG |
| | 190 | | GCT | | GCA | | 382 | | GTG | | C |
| | M2- | 191 | AGGTAGT | 383 | CCTTGTC | | M2- | 575 | CTAGGTA | 767 | GAGTCCTAT |
| | 191 | | AAG | | TAC | | 383 | | AGA | | T |
| | M2- | 192 | TACGGC | 384 | AGACCGA | | M2- | 576 | GATCCAT | 768 | TCCAGTCC |
| | 192 | | GTGA | | ATG | | 384 | | CAT | | AG |

2. A kit for constructing a DNA library, comprising an amplification primer composition, wherein the amplification primer composition comprises a combination of a plurality of amplification primer pairs and a bubble adapter in the method for constructing a DNA library based on an MGI platform according to claim 1.

3. A sequencing library constructed by the method for constructing a DNA library based on an MGI platform according to claim 1.
